(19) United States
(12) Patent Application Publication
(10) Pub. No.: US 2017/0298395 A1
(43) Pub. Date: Oct. 19, 2017

(54) AUTOTROPHIC HYDROGEN BACTERIA AND USES THEREOF

(71) Applicant: Ohio State Innovation Foundation, Columbus, OH (US)

(72) Inventors: F. Robert Tabita, Dublin, OH (US); Richard A. Laguna, Columbus, OH (US); Christopher J. Rocco, Baltimore, OH (US); Sriram Satagopan, Columbus, OH (US); Andrew W. Dangel, Columbus, OH (US); Jon-David Swift Sears, Columbus, OH (US)

(21) Appl. No.: 15/369,039

(22) Filed: Dec. 5, 2016

Related U.S. Application Data
(63) Continuation of application No. 14/001,130, filed on Dec. 9, 2013, now abandoned.
(60) Provisional application No. 61/447,019, filed on Feb. 26, 2011.
(60) Provisional application No. 61/446,773, filed on Feb. 25, 2011.

(51) Int. Cl. C12P 7/16; C12N 9/88; C07K 14/195; C12N 15/52; C12N 15/74

(52) U.S. Cl. CPC C12P 7/16; C12N 15/52; C12N 15/74; C07K 14/195; C12N 9/88; Y02E 50/10

(57) ABSTRACT

In an aspect, the invention relates to compositions and methods production of n-butanol by aerobic hydrogen bacteria. This abstract is intended as a scanning tool for purposes of searching in the particular art and is not intended to be limiting of the present invention.

### CROSS REFERENCE TO RELATED APPLICATION

This application is a Continuation of U.S. Ser. No. 14/001,130 filed Dec. 9, 2013, which is a 371 of International Application No. PCT/US2012/026641 filed Feb. 24, 2012, which claims priority to U.S. Provisional Application No. 61/446,773 filed Feb. 25, 2011, and to U.S. Provisional Application No. 61/447,019 filed Feb. 26, 2011, each of which are incorporated herein fully by reference.

### STATEMENT REGARDING FEDERALLY SPONSORED RESEARCH

This invention was made with government support under DE-AR0000095 awarded by the Advanced Research Projects Agency-Energy (ARPA-E), an agency within the Department of Energy (DOE). The government has certain rights in the invention.

### BACKGROUND

Mankind's reliance on fuel sources is undeniable. Such fuel sources are becoming increasingly limited and difficult to acquire. As fossil fuels are being consumed at an unprecedented rate, the demand for fossil fuels is likely to soon outweigh the available supply.

Therefore, efforts are being made to develop and utilize sources of renewable energy, such as biomass. The use of biomasses including engineered microorganisms to produce new sources of fuel which are not derived from petroleum sources (i.e., biofuel) has emerged as one alternative option. Biofuel is a biodegradable, clean-burning combustible fuel. Therefore, there is a need for an economically- and energy-efficient biofuel and method of making biofuels from renewable energy sources, such as an engineered microorganism.

Despite these efforts, there is still a scarcity of compositions and methods that are economically- and energy-efficient on an industrial or commercial scale. These needs and other needs are satisfied by the present invention.

### SUMMARY

Disclosed herein are isolated aerobic hydrogen bacteria.

Disclosed herein are isolated aerobic bacteria comprising one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof.

Disclosed herein are isolated aerobic hydrogen bacteria comprising one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, or a combination thereof, wherein the aerobic hydrogen bacteria comprising the one or more exogenous nucleic acid molecules is capable of converting CO₂ to n-butanol, and wherein aerobic hydrogen bacteria without the one or more exogenous nucleic acid molecules is incapable of converting CO₂ to n-butanol.

Disclosed herein are isolated aerobic hydrogen bacteria comprising a genetic modification, wherein the genetic modification comprises transformation of the bacteria with one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, or a combination thereof, wherein expression of the polypeptide increases the efficiency of producing n-butanol.

Disclosed herein are isolated aerobic hydrogen bacteria comprising a genetic modification, wherein the genetic modification comprises one or more mutations in a gene encoding a ribulose bisphosphate carboxylase peptide.

Disclosed herein are isolated aerobic hydrogen bacteria comprising one or more mutations in a nucleic acid sequence that encodes a mutated ribulose bisphosphate carboxylase peptide.

Disclosed herein are isolated aerobic hydrogen bacteria comprising a genetic modification, wherein the genetic modification comprises one or more mutations in a gene encoding a ribulose bisphosphate carboxylase peptide

Disclosed herein are isolated aerobic hydrogen bacteria comprising one or more mutations in a nucleic acid sequence that encodes a mutated CbbR peptide.

Disclosed herein are isolated aerobic hydrogen bacteria comprising a genetic modification, wherein the genetic modification comprises one or more mutations in a gene encoding a CbbR peptide. In an aspect, the mutated CbbR peptide is constitutively active. In an aspect, the mutated CbbR peptide is more active than a wild-type CbbR peptide or a non-mutated CbbR peptide.

Disclosed herein are isolated aerobic hydrogen bacteria, wherein one or more endogenous genes is silenced or knocked out.

Disclosed herein are recombinant aerobic hydrogen bacteria, comprising a knockout mutation in gene phaC1 or gene phaC2 (encoding the poly(3-hydroxybutyrate) polymerase enzyme), wherein the knockout mutation decreases the amount of peptide produced in the recombinant aerobic hydrogen bacteria when compared to an aerobic hydrogen bacteria lacking the knockout mutation grown under identical reaction conditions.

Disclosed herein are recombinant aerobic hydrogen bacteria, comprising a knockout mutation in gene ackA or gene ptal, wherein the knockout mutation decreases the amount of peptide produced in the recombinant aerobic hydrogen bacteria when compared to an aerobic hydrogen bacteria lacking the knockout mutation grown under identical reaction conditions.

Disclosed herein are isolated aerobic hydrogen bacteria comprising (i) one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, (ii) a genetic modification, wherein the genetic modification comprises one or more mutations in a gene encoding a ribulose bisphosphate carboxylase peptide, and (iii) a genetic modification, wherein the genetic modification comprises one or more mutations in a gene encoding a CbbR peptide.

Disclosed herein is a method of producing n-butanol, comprising (a) culturing a population of aerobic hydrogen bacteria autotrophically, wherein (i) the aerobic hydrogen bacteria comprise one or more exogenous nucleic acid molecules encoding a naturally occurring polypeptide, (ii) the carbon source comprises CO₂, and (b) recovering the n-butanol from the medium.

Disclosed herein is a method of producing n-butanol, comprising (a) culturing a population of aerobic hydrogen bacteria autotrophically, wherein (i) the aerobic hydrogen bacteria comprises a genetic modification, wherein the genetic modification comprises one or more mutations in a gene encoding a ribulose bisphosphate carboxylase peptide, (ii) the carbon source comprises CO₂, and (b) recovering the n-butanol from the medium.

Disclosed herein is a method of producing n-butanol, comprising (a) culturing a population of aerobic hydrogen bacteria autotrophically, wherein (i) the aerobic hydrogen bacteria comprises a genetic modification, wherein the genetic modification comprises one or more mutations in a gene encoding a CbbR peptide, (ii) the carbon source comprises CO₂, and (b) recovering the n-butanol from the medium.

Disclosed herein is a method of preparing n-butanol, the method comprising culturing engineered aerobic hydrogen in the dark and in a medium comprising oxygen, hydrogen, and carbon dioxide, and isolating the n-butanol.

Disclosed herein is a method of producing n-butanol, the method comprising cultivating aerobic hydrogen bacteria in a medium, wherein the aerobic hydrogen bacteria comprise (i) one or more exogenous genes, (ii) one or more mutations in a nucleic acid sequence that encodes a ribulose bisphosphate carboxylase peptide, or (iii) one or more mutations in a nucleic acid sequence that encodes a CbbR peptide; recovering the aerobic hydrogen bacteria from the medium; and recovering the n-butanol from the medium.

Disclosed herein is a process for preparing n-butanol, the process comprising providing a culture, the culture comprising aerobic hydrogen bacteria comprising (i) one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, (ii) a genetic modification, wherein the genetic modification comprises one or more mutations in a gene encoding a ribulose bisphosphate carboxylase peptide, and (iii) a genetic modification, wherein the genetic modification comprises one or more mutations in a gene encoding a CbbR peptide; culturing the aerobic hydrogen bacteria in the dark and in the presence of oxygen, hydrogen, and carbon dioxide; and recovering the n-butanol from the culture.

Disclosed herein are vectors comprising the disclosed compositions. Disclosed herein are vectors for use in the disclosed method.

Disclosed herein is a vector comprising one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying Figures, which are incorporated in and constitute a part of this specification, illustrate several aspects and together with the description serve to explain the principles of the invention.

FIG. 1 shows genes from C. acetobutylicum (bdhA/bdhB, adhE1/adhE2) for cloning and expression in R. eutropha and R. capsulatus using inducible promoter/vector constructs.

FIG. 2 shows genes encoding butyraldehyde and butanol dehydrogenase activities and their insertion in hydrogen bacteria to allow butyryl-CoA conversion to butanol.

FIG. 3 shows production of recombinant CbbR from R. eutropha in E. coli. Depicted are SDS polyacrylamide electrophoresis gels of extracts prepared from uninduced cells (lane 4) and induced cells (lane 5, showing the high level of recombinant CbbR attained (estimated at or somewhat greater than 20% of the soluble protein). Lanes 2 and 3 contain purified R. eutropha CbbR while lane 1 contains purified R. sphaeroides CbbR.

FIG. 4 shows gel mobility shift assays to show binding of recombinant R. eutropha CbbR to [³²P]-labeled DNA probe. Shown are autoradiograms of labeled probe containing the various combinations of probe, CbbR and potential metabolite effectors. Lanes: (1), probe only; lanes 2-8, probe containing 40 mM CbbR (lane 2), 40 mM CbbR+400 μM RuBP (lane 3), 40 mM CbbR+400 μM Ru5P (lane 4); 40 mM CbbR+400 μM PEP (lane 5), 400 μM NADPH (lane 6), 400 μM ATP (lane 7), 400 μM FBP (lane 8).

FIG. 5 shows SDS polyacrylamide gel electrophoreto-gram of recombinant R. eutropha RubisCO. The cbbLS genes from R. eutropha were expressed in Escherichia coli using a T7 promoter system and purified from crude extracts through nickel affinity and ion exchange columns. The recombinant protein was highly active and routinely isolated with a k_{cat} of 3 to 4 sec⁻¹. Y-axis shows molecular weight standards.

FIGS. 6A-D show phosphorimages of gel mobility shift assays of R. eutropha CbbR binding to a 246 bp chromosomal encoded cbb promoter probe. FIG. 6A shows wild type CbbR, illustrating an enhancement of binding in the presence of RuBP, PEP and ATP, a modest enhancement of binding in the presence of NADPH, and no enhancement of binding in the presence of Ru5P and FBP. FIG. 6B shows CbbR mutants R135C and R154H, illustrating a reduction of binding in the presence of PEP (R135C), or a reduction in the enhancement of binding in the presence of PEP (R154H) compared to wild type CbbR. FIG. 6C shows CbbR mutants R135C and R154H, illustrating a reduction of binding in the presence of RuBP. FIG. 6D shows CbbR mutants R135C and R154H, illustrating a reduction in the enhancement of binding in the presence of ATP compared to wild type CbbR.

FIGS. 7A-C shows phosphorimages of gel mobility shift assays of R. eutropha CbbR binding to a cbb promoter probe. FIG. 7A shows CbbR mutants G98R and R272Q, illustrating an enhancement of binding in the presence of PEP (G98R) similar to wild type CbbR, or a reduction of binding in the presence of PEP (R272Q). FIG. 7B shows CbbR mutants G98R and R272Q, illustrating a modest enhancement of binding in the presence of RuBP (G98R) compared to wild type CbbR, or a reduction of binding in the presence of RuBP (R272Q). FIG. 7C shows CbbR mutants G98R and R272Q, illustrating no enhancement of binding in the presence of ATP (G98R), or a modest enhancement of binding in the presence of ATP (R272Q) compared to wild type CbbR.

FIG. 8 shows a summary of different pathways being tested for butanol production in R. eutropha. The adhE2 gene from C. acetobutylicum is tested with the native R. eutropha genes and using various promoters. The efficiency of this same pathway using all C. acetobutylicum pathway genes in R. eutropha is compared. The final pathway of interest combines genes from E. coli, T. denticola and C. acetobutylicum.

FIG. 9 shows PCR analysis of phaC gene. The wild-type phaC gene is 1436 bp in length (lane 5), while the constructed mutant phaC deletion gene is 863 bp in length. Partial phaC deletion isolates have been created as indicated by the presence of both the wild-type and mutant phaC genes, lanes 1-4. The isolates that only retain the mutant phaC gene are selected.

FIG. 10 shows creation of a CbbR reporter strain (e.g., pVKcbbR) for the isolation of desired mutant CbbR proteins.

FIG. 11 shows growth curves of R. capsulatus SBI/II-complemented with Ralstonia RubisCOs.

FIG. 12 shows gel electrophoresis of phaC1 transcript generated by RT-PCR. Lanes 1 and 2; samples from wild-type R. eutropha grown under rich and poor nitrogen conditions, respectively. Under poor nitrogen conditions, the phaC1 gene is expressed (note 170 bp fragment). Lanes 3 and 4 depict the phaC1 deletion strain grown under the same conditions as above, respectfully; here the phaC1 gene is not expressed (lane 4) under poor nitrogen conditions due to the genomic deletion of this gene in the mutant strain.

FIG. 13 shows a schematic of R. eutropha lacZ reporter strain with endogenous cbbR knocked out on the chromosome complemented with plasmid-borne mutant cbbR.

FIG. 14 shows RubisCO accumulation in R. eutropha cbbR deletion reporter strain complemented with constitutive CbbR mutants, wild type CbbR, or no CbbR. Ten mg of crude extract from each chemoheterotrophically or chemoautotrophically grown culture was separated by SDS-PAGE and subjected to immunoblot analysis using antibodies directed against form I large subunit of RubisCO. 1) no CbbR, 2) wild type CbbR, 3) E87K/G242S, 4) A167V, 5) D148N, 6) P221S/T299I, 7) A117V, 8) D144N, 9) G125S/V265M, 10) A117V. Lanes 1-9: cells were grown under chemoheterotrophic conditions, and in lane 10, cells were grown under chemoautotrophic conditions.

FIG. 15 shows genomic and megaplasmid (pHG1) loci around the cbbLS genes of Ralstonia, with the regions to be deleted marked.

FIG. 16 shows a comparison of the generations per hour of R. eutropha H16 (wild-type) with the growth rates of two adaptation isolates (X1, F23) in complex media with increasing concentrations of butanol. Growth of wild-type was not seen at concentrations above 0.6% butanol (v/v).

FIG. 17 shows structure of RubisCO showing classical CO₂ fixation problem in aerobic organisms.

FIG. 18 shows the structure of R. eutropha RubisCO (yellow) showing the position of residues Ala380 and Tyr347 (red) in a hydrophobic region near the active site (marked by Ser381 in blue and CABP in black).

FIG. 19 shows growth phenotypes of R. capsulatus SB I/II-complemented with RubisCO genes from Synechococcus (form I) or R. rubrum (form II) or A. fulgidus or M. acetovorans (form III).

FIG. 20 shows photoautotrophic growth profiles of R. capsulatus SBI/II-complemented with different RubisCO enzymes, in liquid minimal medium bubbled with a 5% CO₂/95% H₂ in light.

FIG. 21 shows RT-PCR of cbb transcripts isolated from the chemoautotrophically grown Ralstonia eutropha cbbR deletion strain complemented with CbbR constitutive mutants or wild type CbbR, illustrating an increase in transcriptional activity from the cbb promoter when activated by CbbR constitutive mutants relative to activation by wild type CbbR. RNA was isolated when cells were at an optical density of 0.2. One ng of RNA was used for RT-PCR analysis from each sample. Equal amounts of each RT-PCR reaction were loaded on a 2% agarose gel. The PCR product is a 341 bp fragment amplified from the cDNA of the cbbL transcript. Lane 1: CbbR-A117V; lane 2: CbbR-D144N; lane 3: CbbR-A167V; lane 4: CbbR-wild type; lane 5: negative control, RNA from samples A117V, D144N and A167V using no reverse transcriptase but using Taq DNA polymerase to ensure there is no DNA contamination in the RNA; lane 6: negative control, RNA from the wild type sample; lane 7: H16 strain (wild type strain, no complementation of CbbR). Chemoautotrophic growth conditions: 5% CO₂, 10% O₂ (as compressed air), 45% H₂ and ˜40% N₂.

FIG. 22 shows RT-PCR of cbb transcripts isolated from the chemoautotrophically grown Ralstonia eutropha cbbR deletion strain complemented with CbbR constitutive mutants or wild type CbbR, illustrating an increase in transcriptional activity from the cbb promoter when activated by CbbR constitutive mutants relative to activation by wild type CbbR. RNA was isolated when cells were at an optical density of 0.2. One ng of RNA was used for RT-PCR analysis from each sample. Equal amounts of each RT-PCR reaction were loaded on a 2% agarose gel. The PCR product is a 341 bp fragment amplified from the cDNA of the cbbL transcript. Lane 1: CbbR-D144N; lane 2: CbbR-A167V; lane 3: CbbR-wild type; lane 4: H16 strain (wild type strain, no complementation of CbbR); lane 5: negative control, RNA from sample D144N using no reverse transcriptase but using Taq DNA polymerase to ensure there is no DNA contamination in the RNA; lane 6: negative control, RNA sample from A176V; lane 7: negative control, RNA from the wild type sample. Chemoautotrophic growth conditions: 5% CO₂, 10% O₂ (as compressed air), 45% H₂ and ˜40% media at 30° C.

FIG. 23 shows butanol synthesis and different pathways involved in butanol production.

FIG. 24 shows the pathway and genes involved in polyhydroxybutyrate (PHB) synthesis. Deletion of phaC gene shifts carbon flow to butyryl-CoA to optimize butanol production.

FIG. 25 shows the CbbR constitutive mutants from R. eutropha.

FIG. 26 shows the structure of RubisCO, showing areas of structural strains for CO₂ conversion in aerobic growth conditions.

FIG. 27 show growth phenotypes of Ralstonia grown under chemoheterotrophic and organoautotrophic conditions.

FIG. 28 shows growth phenotypes of normal and mutant RubisCO with and without the presences of oxygen. In FIGS. 6(a) and 6(c): sections 2, 3, and 4 represent cells containing normal RubisCO, and sections 1, and 5 represent cells containing mutant RubisCO. FIGS. 6(a) and 6(b) show growth without the presence of oxygen. FIGS. 6(c) and 6(d) show growth in the presence of oxygen.

FIG. 29 shows chemoheterotrophic growth of R. eutropha, showing R. eutropha reporter strain with mutagenized cbbR with blue colonies have activated the cbb promoter under repressive conditions.

FIG. 30 shows insertion of bdhA and bdhB into pRPS-MCS3 vector. Expression of bdhAB is under the control of the R. rubrum cbbR gene.

FIG. 31 shows insertion of adhE1 into pRPS-MCS3 vector. Expression of adhE1 is under the control of the R. rubrum cbbR gene.

FIG. 32 shows a suicide vector with kanamycin.

FIG. 33 shows the broad host vector showing the R. rubrum cbbM promoter, which is regulated in response to CO₂ fixation and cellular redox.

FIG. 34 shows the vector map for pJQ200mp18 comprising atoB crt ter adhE2 fadB.

FIG. 35 shows the vector map for pJQ200mp18 comprising atoB hbd crt ter adhE2.

FIG. 36 shows the vector map for pJQ200mp18 comprising atoB hbd crt ter Ma2507.

FIG. 37 shows the vector map for pJQ200mp18 comprising atoB hbd crt ter mhpF fucO.

FIG. 38 shows the vector map for pJQ200mp18 comprising hbd crt ter mhpF fucO yqeF.

FIG. 39 shows the vector map for pRPSMCS3.

FIG. 40 shows the vector map for pBBR1MCS3ptac.

FIG. 41 shows the vector map for pBBR1MCS3.

FIG. 42 shows the vector map for pBBR1MCS3pBADaraC.

FIG. 43 shows constitutive CbbR molecule cbb gene expression activity under conditions where CO₂ is sole carbon source.

FIG. 44 shows doubling times for CO₂-grown Ralstonia eutropha cbbR deletion reporter strain complemented with CbbR constitutive mutants.

FIG. 45 shows enzyme activity as NAD⁺is reduced to NADH in R. eutropha incubated in carbon free MOPS-Repaske's medium inside sealed serum bottles containing mixtures of H₂, CO₂, and air at varying ratios.

FIG. 46 shows hydrogenase assay response for R. eutropha grown overnight on TSB.

Additional advantages of the invention will be set forth in part in the description that follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. Definitions

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The word “or” as used herein means any one member of a particular list and also includes any combination of members of that list.

The term “cell” as used herein also refers to individual microbial cells, or cultures derived from such cells. A “culture” refers to a composition comprising isolated cells of the same or a different type.

It will be apparent to those of skill in the art that a nucleic acid existing among hundreds to millions of other nucleic acid molecules within, for example, cDNA or genomic libraries, or gel slices containing a genomic DNA restriction digest is not to be considered an isolated nucleic acid.

As used herein, the term “isolated” when used in reference to an aerobic hydrogen bacteria or microbial organism or microorganism is intended to mean aerobic hydrogen bacteria or other microbial organism or microorganism that is substantially free of at least one component as the referenced aerobic hydrogen bacteria or other microbial organism or microorganism is found in nature. For example, the term includes n aerobic hydrogen bacteria that is removed from some or all components as it is found in its natural environment. The term also includes an aerobic hydrogen bacteria that is removed from some or all components as the aerobic hydrogen bacteria is found in non-naturally occurring environments. Therefore, an isolated aerobic hydrogen bacteria is partly or completely separated from other substances as it is found in nature or as it is grown, stored or subsisted in non-naturally occurring environments. Specific examples of isolated aerobic hydrogen bacteria include partially pure aerobic hydrogen bacteria, substantially pure aerobic hydrogen bacteria and aerobic hydrogen bacteria cultured in a medium that is non-naturally occurring.

In accordance with the present invention, an “isolated nucleic acid molecule” is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene. An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein or domain of a protein.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus), or the genomic DNA of a prokaryote or eukaryote. In addition, a non-naturally-occurring nucleic acid can include a nucleic acid molecule that is part of a hybrid or fusion nucleic acid sequence.

Preferably, an isolated nucleic acid molecule or nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on the genes product's biological activity as described herein.

The term “exogenous” as used herein with reference to a nucleic acid and a particular organism refers to any nucleic acid that does not originate from that particular organism as found in nature. Thus, non-naturally-occurring nucleic acid is considered to be exogenous to a cell once introduced into the organism. It is important to note that non-naturally-occurring nucleic acid can contain nucleic acid sequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a cell once introduced into the cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. Nucleic acid that is naturally-occurring can be exogenous to a particular organism. For example, an entire chromosome isolated from a cell of organism X is an exogenous nucleic acid with respect to a cell of organism Y once that chromosome is introduced into oganism's cell.

“Exogenous” as it is used herein is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism.

Therefore, as used herein, the term “endogenous” refers to a referenced molecule naturally present in the host. Similarly, the term when used in reference to expression of a nucleic acid refers to expression of a nucleic acid naturally present within the microbial organism.

As used herein, the term “heterologous” refers to a molecule or activity derived from a source other than the referenced species whereas “homologous” refers to a molecule or activity derived from the host microbial organism. Accordingly, exogenous expression of an encoding nucleic acid of the invention can utilize either or both a heterologous or homologous encoding nucleic acid.

As used herein, “ribosome binding site” or RBS is a segment of the 5′ (upstream) part of an mRNA molecule that binds to the ribosome to position the message correctly for the initiation of translation. As known to the art, the RBS controls the accuracy and efficiency with which the translation of mRNA begins. In prokaryotes, the ribosome binding site (RBS), which promotes efficient and accurate translation of mRNA, is called the Shine-Dalgarno sequence. This purine-rich sequence of 5′ UTR is complementary to the UCCU core sequence of the 3′-end of 16S rRNA (located within the 30S small ribosomal subunit). Various Shine-Dalgarno sequences are known to the art. These sequences lie about 10 nucleotides upstream from the AUG start codon. Activity of a RBS can be influenced by the length and nucleotide composition of the spacer separating the RBS and the initiator AUG.

As used herein, the amino acid abbreviations are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

“Peptide” as used herein refers to any peptide, oligopeptide, polypeptide, gene product, expression product, or protein. For example, a peptide can be an enzyme. A peptide is comprised of consecutive amino acids. The term “peptide” encompasses naturally occurring or synthetic molecules.

An “isolated peptide”, such as an isolated ribulose bisphosphate carboxylase (RubisCO), according to the present invention, is a protein that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated ribulose bisphosphate carboxylase of the present invention is produced recombinantly. For example, an “exogenous isolated ribulose bisphosphate carboxylase” refers to a ribulose bisphosphate carboxylase (including a homologue of a naturally occurring acetolactate synthase) from a source other than the host or that has been otherwise produced from the knowledge of the structure (e.g., sequence) of a naturally occurring isolated ribulose bisphosphate carboxylase from a source other than the host.

In general, the biological activity or biological action of a peptide refers to any function(s) exhibited or performed by the peptide that is ascribed to the naturally occurring form of the peptide as measured or observed in vivo (i.e., in the natural physiological environment of the protein) or in vitro (i.e., under laboratory conditions). For example, a biological activity of a ribulose bisphosphate carboxylase includes ribulose bisphosphate carboxylase enzymatic activity.

Modifications of a peptide, such as in a homologue or mimetic, may result in peptides having the same biological activity as the naturally occurring peptide, or in peptides having decreased or increased biological activity as compared to the naturally occurring peptide. Modifications which result in a decrease in peptide expression or a decrease in the activity of the peptide, can be referred to as inactivation (complete or partial), down-regulation, or decreased action of a peptide. Similarly, modifications that result in an increase in peptide expression or an increase in the activity of the peptide can be referred to as amplification, overproduction, activation, enhancement, up-regulation or increased action of a peptide.

The term “enzyme” as used herein refers to any peptide that catalyzes a chemical reaction of other substances without itself being destroyed or altered upon completion of the reaction. Typically, a peptide having enzymatic activity catalyzes the formation of one or more products from one or more substrates. Such peptides can have any type of enzymatic activity including, without limitation, the enzymatic activity or enzymatic activities associated with enzymes such as those disclosed herein.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or can not occur, and that the description includes instances where said event or circumstance occurs and instances where it does not.

As used herein, the term “analog” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

As used herein, “homolog” or “homologue” refers to a polypeptide or nucleic acid with homology to a specific known sequence. Specifically disclosed are variants of the nucleic acids and polypeptides herein disclosed which have at least 40, 41, 42, 43, 44, 45, 46, 47, 48, 49, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated or known sequence. Those of skill in the art readily understand how to determine the homology of two or more proteins or two or more nucleic acids. For example, the homology can be calculated after aligning the two sequences so that the homology is at its highest level. It is understood that one way to define any variants, modifications, or derivatives of the disclosed genes and proteins herein is through defining the variants, modification, and derivatives in terms of homology to specific known sequences.

As used herein, “EC₅₀,” is intended to refer to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest.

As used herein, “EC₅₀,” is intended to refer to the concentration or dose of a substance (e.g., a compound or a drug) that is required for 50% inhibition or diminution of a biological process, or component of a process, including a protein, subunit, organelle, ribonucleoprotein, etc. IC₅₀ also refers to the concentration or dose of a substance that is required for 50% inhibition or diminution in vivo, as further defined elsewhere herein. Alternatively, IC₅₀ also refers to the half maximal (50%) inhibitory concentration (IC) or inhibitory dose of a substance. The response can be measured in an in vitro or in vivo system as is convenient and appropriate for the biological response of interest.

As used herein, the term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a nucleic acid construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

As used herein, with respect to nucleic acid molecules, a “transcriptional control element” or “control element” refers to those elements in an expression vector or construct that interact with host cellular proteins to carry out transcription and translation (e.g., non-translated regions of the vector and/or construct, enhancers, promoters, 5′ and 3′ untranslated regions). Such a control element may vary in their strength and specificity. Depending on the vector system and host utilized, any number of suitable transcription and translation elements, including constitutive and inducible promoters, may be used. A control element may be inserted into a somatic cell. A control element may be targeted to a chromosomal locus where it will effect expression of a particular gene that is responsible for expression of a protein product. The art is familiar with control elements generally as well as specific eukaryotic and prokaryotic promoters and enhancers. “Transcriptional control element” or “Control element”are used interchangeably.

The term “sequence of interest” or “gene of interest” can mean a nucleic acid sequence (e.g., a therapeutic gene), that is partly or entirely heterologous, i.e., foreign, to a cell into which it is introduced. The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be cDNA, DNA, or mRNA.

The term “sequence of interest” or “gene of interest” can also mean a nucleic acid sequence that is partly or entirely complementary to an endogenous gene of the cell into which it is introduced. For example, the sequence of interest can be micro RNA, shRNA, or siRNA. A “sequence of interest” or “gene of interest” can also include one or more transcriptional regulatory sequences and any other nucleic acid, such as introns, that may be necessary for optimal expression of a selected nucleic acid. A “protein of interest” means a peptide or polypeptide sequence (e.g., a therapeutic protein), that is expressed from a sequence of interest or gene of interest.

A “gene transfer construct” refers to a nucleic acid sequence that is typically used in conjunction with other lentiviral or trans-lentiviral vector system vectors to produce viral particles, e.g., so that the viral particles can then transduce a target cell of interest.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “transformation” and “transfection” mean the introduction of a nucleic acid, e.g., an expression vector, into a recipient cell including introduction of a nucleic acid to the chromosomal DNA of said cell.

The art is familiar with methods of silencing or knocking out genes. For example, short interfering RNAs (siRNAs), also known as small interfering RNAs, are double-stranded RNAs that can induce sequence-specific post-transcriptional gene silencing, thereby decreasing gene expression. siRNAs can be of various lengths as long as they maintain their function. In some examples, siRNA molecules are about 19-23 nucleotides in length, such as at least 21 nucleotides, and for example at least 23 nucleotides. siRNAs can effect the sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate transcription or translation, for example, by decreasing expression of phaA, phaB1, phaC1, phaC2, or a combination thereof. SiRNAs can also be used to modulate transcription or translation of other genes of interest as well. (See., e.g., Invitrogen's BLOCK-IT™ RNAi Designer (https://maidesigner.invitrogen.com/maiexpress).

shRNA (short hairpin RNA) is a DNA molecule that can be cloned into expression vectors to express siRNA (typically 19-29 nt RNA duplex) for RNAi interference studies. shRNA has the following structural features: a short nucleotide sequence ranging from about 19-29 nucleotides derived from the target gene, followed by a short spacer of about 4-15 nucleotides (i.e., loop) and about a 19-29 nucleotide sequence that is the reverse complement of the initial target sequence.

Generally, the term “antisense” refers to a nucleic acid molecule capable of hybridizing to a portion of an RNA sequence (such as mRNA) by virtue of some sequence complementarity. The antisense nucleic acids disclosed herein can be oligonucleotides that are double-stranded or single-stranded, RNA or DNA or a modification or derivative thereof, which can be directly administered to a cell (for example by administering the antisense molecule to the subject), or which can be produced intracellularly by transcription of exogenous, introduced sequences (for example by administering to the subject a vector that includes the antisense molecule under control of a promoter). In an aspect, antisense oligonucleotides or molecules are designed to interact with a target nucleic acid molecule (i.e., phaA, phaB1, phaC1, and/or phaC2) through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10-10, or 10-12. In an aspect, the antisense oligonucleotide can be conjugated to another molecule, such as a peptide, hybridization triggered cross-linking agent, transport agent, or hybridization-triggered cleavage agent. Antisense oligonucleotides can include a targeting moiety that enhances uptake of the molecule by host cells. The targeting moiety can be a specific binding molecule, such as an antibody or fragment thereof that recognizes a molecule present on the surface of the host cell. Antisense molecules can be generated by utilizing the Antisense Design algorithm of Integrated DNA Technologies, Inc., available at http://www.idtdna.com/Scitools/Applications/AntiSense/Antisense.aspx/.

A “genetic modification” as used herein refers to the direct human manipulation of a nucleic acid using modern DNA technology. For example, genetic manipulation can involve the introduction of exogenous nucleic acids into an organism or alterting or modifying an endogenous nucleic acid sequence present in the organism. For example, a genetic modification can be insertion of a nucleotide sequence into the genomic DNA of an aerobic hydrogen bacteria. A genetic modification can also be a deletion or disruption of a polynucleotide that encodes, or regulates production of an endogenous or exogenous gene. A genetic modification can result in the mutation of a nucleic acid or polypeptide sequence.

A “mutation” as used herein refers to changes to or alterations of a nucleic acid sequence or polypeptide sequence.

As used herein, a “mutant” can be an aerobic hydrogen bacteria or microbial organism or microorganism, or new genetic character arising or resulting from mutation. For example, a “mutant” can be a subject that has characteristics resulting from chromosomal alteration, a an aerobic hydrogen bacteria or microbial organism or microorganism that has undergone mutation or a an aerobic hydrogen bacteria or microbial organism or microorganism tending to undergo or resulting from mutation. For example, a mutant can be an aerobic hydrogen bacteria or microbial organism or microorganism that comprises a mutation in the ribulose bisphosphate carboxylase peptide.

By “modulate” is meant to alter, by increase or decrease.

As used herein, a “modulator” can mean a composition that can either increase or decrease the expression or activity of a gene or gene product such as a peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition. For example, a “candidate modulator” can be an active agent or a therapeutic agent.

“Differential expression” or “different expression” or “altered expression” can be use interchangeably herein. “Differential expression” or “different expression” or “altered expression” as used herein refers to the change in expression levels of genes, and/or proteins encoded by said genes, in cells, tissues, organs or systems upon exposure to an agent. As used herein, “differential expression” or “different expression” or “altered expression” includes differential transcription and translation, as well as message stabilization. Differential gene expression encompasses both up- and down-regulation of gene expression.

“Naturally occurring” refers to an endogenous chemical moiety, such as a polynucleotide or polypeptide sequence, i.e., one found in nature. Processing of naturally occurring moieties can occur in one or more steps, and these terms encompass all stages of processing including, but not limited to the metabolism of a non-active compound to an active compound. Conversely, a “non-naturally occurring” moiety refers to all other moieties, e.g., ones which do not occur in nature, such as recombinant polynucleotide sequences and non-naturally occurring polypeptide.

“Purify” and any form such as “purifying” refers to the state in which a substance or compound or composition is in a state of greater homogeneity than it was before. It is understood that as disclosed herein, something can be, unless otherwise indicated, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% pure. For example, if a given composition A was 90% pure, this would mean that 90% of the composition was A, and that 10% of the composition was one or more things, such as molecules, compounds, or other substances. For example, if a disclosed aerobic hydrogen bacteria, for example, produces 35% n-butanol, this could be further “purified” such that the final composition was greater than 90% n-butanol. Unless otherwise indicated, purity will be determined by the relative “weights” of the components within the composition. It is understood that unless specifically indicated otherwise, any of the disclosed compositions can be purified as disclosed herein.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compositions

Aerobic hydrogen bacteria can be utilized for the efficient bioconversion of carbon dioxide to butanol. To improve the catalytic efficiency and oxygen sensitivity of the CO₂ assimilatory enzyme RubisCO, several modifications in the basic metabolism of the organism are performed. Furthermore, these modifications also enhance the ability of the organism to express the CO₂ fixation genes, which increase conversion of CO₂ to organic carbon and ultimately generate higher levels of butanol. The master regulator protein, CbbR, can also be modified to enhance gene expression. These improvements in upstream carbon assimilation are coupled to the removal of competing downstream carbon metabolic pathways. Finally, exogenous genes that encode enzymes that contribute to butanol synthesis can be inserted into the hydrogen bacteria, thereby resulting in improved carbon assimilatory properties.

For example, RubisCO catalyzes the CO₂ fixation reaction of the disclosed aerobic hydrogen bacteria. The fixation reaction can be inefficient and can be inhibited by the presence of oxygen. CbbR belongs to a ubiquitous class of regulators that regulate many important processes in bacteria, called LysR-type transcriptional regulators (or LTTRs). Often LTTRs require either positive or negative metabolites (effectors) in order for these proteins to control gene transcription. CbbR must first be activated by positive effector before genes important for CO₂ fixation are transcribed.

Disclosed herein are isolated aerobic hydrogen bacteria as well as genetically modified micoorganisms.

Disclosed herein are isolated aerobic bacteria comprise one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof.

In an aspect, the aerobic hydrogen bacteria disclosed herein can oxidize hydrogen (H) for energy and can derive carbon from carbon dioxide (CO₂), both in the presence of oxygen (O). In an aspect, the aerobic hydrogen bacteria disclosed herein are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, a culture comprising a plurality of the aerobic hydrogen bacteria produce or secrete n-butanol. In an aspect, the aerobic hydrogen bacteria disclosed herein produces n-butanol when cultured in the presence of oxygen, hydrogen, and carbon dioxide and in the dark. In an aspect, the aerobic hydrogen bacteria are isolated.

In an aspect, the disclosed aerobic hydrogen bacteria comprise crt, bcd, eftA, eftB, hbd, and adhE2. In an aspect, the disclosed aerobic hydrogen bacteria comprise atoB, hbd, crt, ter, and adhE2 In an aspect, the disclosed aerobic hydrogen bacteria comprise atoB, hbd, crt, ter, mhpF, and fucO. In an aspect, the disclosed aerobic hydrogen bacteria comprise hbd, crt, ter, mhpF, fucO, and yqeF. In an aspect, the disclosed aerobic hydrogen bacteria comprise atoB, hbd, crt, ter, and Ma2507. In an aspect, the disclosed aerobic hydrogen bacteria comprise atoB, crt, ter, adheE2, and fadB.

In an aspect, the one or more exogenous nucleic acid molecules disclosed here is operably linked to a control element. In an aspect, the control element is a promoter. In an aspect, the promoter is constitutively active, or inducibly active, or tissue-specific, or development stage-specific. In an aspect, the promoter is cbbL (native), cbbL (constitutive), lac, tac, pha, cbbM, pBAD, or pseudomonas syringae. In an aspect, the cbbL (native) promoter is a R. eutropha promoter. In an aspect, the cbbL (native) promoter comprises SEQ ID NO: 29. In an aspect, the cbbL (constitutive) is a R. eutropha promoter. In an aspect, the cbbL (constitutive) promoter comprises SEQ ID NO: 30. In an aspect, the lac promoter is an E. coli promoter. In an aspect, the lac promoter comprises SEQ ID NO: 31. In an aspect, the tac promoter is a synthetic promoter. In an aspect, the tac promoter is an E. coli promoter. In an aspect, the tac promoter comprises SEQ ID NO: 32. In an aspect, the pha promoter is a R. eutropha promoter. In an aspect, the pha promoter comprises SEQ ID NO: 33. In an aspect, the cbbM promoter is a Rhodosporilium rubrum promoter. In an aspect, the cbbM promoter comprises SEQ ID NO: 34. In an aspect, the pBAD promoter is an arabinose inducible promoter. In an aspect, the pBAD promoter comprises SEQ ID NO: 35.

In an aspect, the aerobic hydrogen bacteria further comprise one or more optimized ribosome binding sites.

Disclosed herein are aerobic hydrogen bacteria comprise one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, wherein the aerobic hydrogen bacteria comprising the one or more exogenous nucleic acid molecules is capable of converting CO₂ to n-butanol, and wherein aerobic hydrogen bacteria without the one or more exogenous nucleic acid molecules is incapable of converting CO₂ to n-butanol.

The aerobic hydrogen bacteria disclosed herein can oxidize hydrogen (H) for energy and can derive carbon from carbon dioxide (CO₂), both in the presence of oxygen (O). In an aspect, the aerobic hydrogen bacteria disclosed herein are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, a culture comprising a plurality of the aerobic hydrogen bacteria produce or secrete n-butanol. In an aspect, the aerobic hydrogen bacteria disclosed herein produces n-butanol when cultured in the presence of oxygen, hydrogen, and carbon dioxide and in the dark. In an aspect, the aerobic hydrogen bacteria are isolated.

In an aspect, the disclosed aerobic hydrogen bacteria comprise crt, bcd, eftA, eftB, hbd, and adhE2 In an aspect, the disclosed aerobic hydrogen bacteria comprise atoB, hbd, crt, ter, and adhE2. In an aspect, the disclosed aerobic hydrogen bacteria comprise atoB, hbd, crt, ter, mhpF, and fucO. In an aspect, the disclosed aerobic hydrogen bacteria comprise hbd, crt, ter, mhpF, fucO, and yqeF. In an aspect, the disclosed aerobic hydrogen bacteria comprise atoB, hbd, crt, ter, and Ma2507. In an aspect, the disclosed aerobic hydrogen bacteria comprise atoB, crt, ter, adheE2, and fadB.

In an aspect, the one or more exogenous nucleic acid molecules disclosed here is operably linked to a control element. In an aspect, the control element is a promoter. In an aspect, the promoter is constitutively active, or inducibly active, or tissue-specific, or development stage-specific. In an aspect, the promoter is cbbL (native), cbbL (constitutive), lac, tac, pha, cbbM, pBAD, or pseudomonas syringae. In an aspect, the cbbL (native) promoter is a R. eutropha promoter. In an aspect, the cbbL (native) promoter comprises SEQ ID NO: 29. In an aspect, the cbbL (constitutive) is a R. eutropha promoter. In an aspect, the cbbL (constitutive) promoter comprises SEQ ID NO: 30. In an aspect, the lac promoter is an E. coli promoter. In an aspect, the lac promoter comprises SEQ ID NO: 31. In an aspect, the tac promoter is a synthetic promoter. In an aspect, the tac promoter is an E. coli promoter. In an aspect, the tac promoter comprises SEQ ID NO: 32. In an aspect, the pha promoter is a R. eutropha promoter. In an aspect, the pha promoter comprises SEQ ID NO: 33. In an aspect, the cbbM promoter is a Rhodosporilium rubrum promoter. In an aspect, the cbbM promoter comprises SEQ ID NO: 34. In an aspect, the pBAD promoter is an arabinose inducible promoter. In an aspect, the pBAD promoter comprises SEQ ID NO: 35.

In an aspect, the aerobic hydrogen bacteria further comprise one or more optimized ribosome binding sites.

Disclosed herein are aerobic hydrogen bacteria comprise a genetic modification, wherein the genetic modification comprises transformation of the aerobic hydrogen bacteria with one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, wherein expression of the polypeptide increases the efficiency of producing n-butanol.

In an aspect, the aerobic hydrogen bacteria disclosed herein can oxidize hydrogen (H) for energy and can derive carbon from carbon dioxide (CO₂), both in the presence of oxygen (O). In an aspect, the aerobic hydrogen bacteria disclosed herein are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, a culture comprising a plurality of the aerobic hydrogen bacteria produce or secrete n-butanol. In an aspect, the aerobic hydrogen bacteria disclosed herein produces n-butanol when cultured in the presence of oxygen, hydrogen, and carbon dioxide and in the dark. In an aspect, the aerobic hydrogen bacteria is isolated.

In an aspect, the disclosed aerobic hydrogen bacteria comprise crt, bcd, eftA, eftB, hbd, and adhE2 In an aspect, the disclosed aerobic hydrogen bacteria comprise atoB, hbd, crt, ter, and adhE2. In an aspect, the disclosed aerobic hydrogen bacteria comprise atoB, hbd, crt, ter, mhpF, and fucO. In an aspect, the disclosed aerobic hydrogen bacteria comprise hbd, crt, ter, mhpF, fucO, and yqeF. In an aspect, the disclosed aerobic hydrogen bacteria comprise atoB, hbd, crt, ter, and Ma2507. In an aspect, the disclosed aerobic hydrogen bacteria comprise atoB, crt, ter, adheE2, and fadB.

In an aspect, the one or more exogenous nucleic acid molecules disclosed here is operably linked to a control element. In an aspect, the control element is a promoter. In an aspect, the promoter is constitutively active, or inducibly active, or tissue-specific, or development stage-specific. In an aspect, the promoter is cbbL (native), cbbL (constitutive), lac, tac, pha, cbbM, pBAD, or pseudomonas syringae. In an aspect, the cbbL (native) promoter is a R. eutropha promoter. In an aspect, the cbbL (native) promoter comprises SEQ ID NO: 29. In an aspect, the cbbL (constitutive) is a R. eutropha promoter. In an aspect, the cbbL (constitutive) promoter comprises SEQ ID NO: 30. In an aspect, the lac promoter is an E. coli promoter. In an aspect, the lac promoter comprises SEQ ID NO: 31. In an aspect, the tac promoter is a synthetic promoter. In an aspect, the tac promoter is an E. coli promoter. In an aspect, the tac promoter comprises SEQ ID NO: 32. In an aspect, the pha promoter is a R. eutropha promoter. In an aspect, the pha promoter comprises SEQ ID NO: 33. In an aspect, the cbbM promoter is a Rhodosporilium rubrum promoter. In an aspect, the cbbM promoter comprises SEQ ID NO: 34. In an aspect, the pBAD promoter is an arabinose inducible promoter. In an aspect, the pBAD promoter comprises SEQ ID NO: 35.

In an aspect, the aerobic hydrogen bacteria further comprise one or more optimized ribosome binding sites.

Disclosed herein are aerobic hydrogen bacteria comprising one or more mutations in a nucleic acid sequence that encodes an endogenous peptide. As used herein, a specific notation will be used to denote certain types of mutations. All notations referencing a nucleotide or amino acid residue will be understood to correspond to the residue number of the wild-type nucleic acid sequence or polypeptide sequence. For example, disclosed herein are aerobic hydrogen bacteria comprising one or more mutations in a nucleic acid sequence that encodes a mutated ribulose bisphosphate carboxylase peptide. Also disclosed herein are aerobic hydrogen bacteria comprising one or more mutations in a nucleic acid sequence that encodes a mutated CbbR peptide. All notations referencing a nucleotide or amino acid residue of a ribulose bisphosphate carboxylase will be understood to correspond to the amino acid residue number of the wild-type ribulose bisphosphate carboxylase amino acid sequence set forth at SEQ ID NO: 24. All notations referencing a nucleotide or amino acid residue of a CbbR will be understood to correspond to the amino acid residue number of the wild-type CbbR amino acid sequence set forth at SEQ ID NO: 1. Thus, for example, the notation “L79F” when used in the context of a polypeptide sequence will be used to indicate that the amino acid leucine at position 79 has been replaced with phenylalanine

The amino acid sequence for wild-type ribulose bisphosphate carboxylase (R. eutropha) (486 amino acids) is as follows:

MNAPESVQAK PRKRYDAGVM
KYKEMGYWDG DYEPKDTDLL ALFRITPQDG VDPVEAAAAV
AGESSTATWT VVWTDRLTAC DMYRAKAYRV DPVPNNPEQF
FCYVAYDLSL FEEGSIANLT ASIIGNVFSF KPIKAARLED
MRFPVAYVKT FAGPSTGIIV ERERLDKFGR PLLGATTKPK
LGLSGRNYGR VVYEGLKGGL DFMKDDENIN SQPFMHWRDR
FLFVMDAVNK ASAATGEVKG SYLNVTAGTM EEMYRRAEFA
KSLGSVVIMI DLIVGWTCIQ SMSNWCRQND MILHLHRAGH
GTYTRQKNHG VSFRVIAKWL RLAGVDHMHT GTAVGKLEGD
PLTVQGYYNV CRDAYTHTDL TRGLFFDQDW ASLRKVMPVA
SGGIHAGQMH QLIHLFGDDV VLQFGGGTIG HPQGIQAGAT
ANRVALEAMV LARNEGRDIL NEGPEILRDA ARWCGPLRAA
LDTWGDISFN YTPTDTSDFA PTASVA.

The amino acid sequence for wild-type CbbR (R. eutropha) (317 amino acids) is as follows:

MQVKQLESVV GMALFERVKG QLTLTEPGDR LLHHASRILG
EVKDAEEGLQ AVKDVEQGSI TIGLISTSKY FAPKLLAGFT
ALHPGVDLRI AEGNRETLLR MSSFLRALTL RQLQIFVTVA
RHASFVRAAE ELHLTQPAVS LLQDNAIDLA LMGRPPRELD
AVSEPIAAHP HVLVASPRHP LHDAKGFDLQ ELRHETFLLR
EPGSGTRTVA EYMFRDHLFT PAKVITLGSN ETIKQAVMAG
MGISLLSLHT LGLELRTGEI GLLDVAGTPI ERIWHVAHMS
SKRLSPASES CRAYLLEHTA EFLGREYGGL MPGRRVA.

Disclosed herein are aerobic hydrogen bacteria comprising a genetic modification, wherein the genetic modification comprises one or more mutations in a gene encoding a ribulose bisphosphate carboxylase peptide. In an aspect, the mutated ribulose bisphosphate carboxylase peptide increases the efficiency of the protein to fix CO₂ In an aspect, the mutated ribulose bisphosphate carboxylase peptide decreases the sensitivity of the protein to O₂. In an aspect, the ribulose bisphosphate carboxylase peptide both increases the efficiency of the protein to fix CO₂ and decreases the sensitivity of the protein to O₂.

In an aspect, the disclosed aerobic hydrogen bacteria comprising a genetic modification, wherein the genetic modification comprises one or more mutations in a gene encoding a ribulose bisphosphate carboxylase peptide, are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the disclosed aerobic hydrogen bacteria comprising a genetic modification, wherein the genetic modification comprises one or more mutations in a gene encoding a ribulose bisphosphate carboxylase peptide, produce n-butanol when cultured in the presence of oxygen, hydrogen, and carbon dioxide and in the dark. In an aspect, the aerobic hydrogen bacteria are isolated.

In an aspect, the mutated ribulose bisphosphate carboxylase peptide of the aerobic hydrogen bacteria is mutated. In an aspect, the mutated ribulose bisphosphate carboxylase peptide of the aerobic hydrogen bacteria is mutated in such a way that it results in a codon change in the wild-type sequence. For example, disclosed herein are aerobic hydrogen bacteria comprising a codon change in SEQ ID NO: 24. In an aspect, the codon change is from GGC to GGT at position 264. In an aspect, the codon change is from TCG to ACC at position 265. In an aspect, the change is S265T (SEQ ID NO: 25). In an aspect, the codon change is from GAC to GAT at position 271. In an aspect, the codon change is from GTG to GGC at position 274. In an aspect, the change is V274G (SEQ ID NO: 26). In an aspect, the codon change is from TAC to GTC at position 347. In an aspect, the change is Y347V (SEQ ID NO: 27). In an aspect, the codon change is from GCC to GTC at position 380. In an aspect, the change is A380V (SEQ ID NO: 28). In an aspect, the mutated ribulose bisphosphate carboxylase peptide comprises a combination of codon changes selected from the following: from GGC to GGT at position 264, from TCG to ACC at position 265, from GAC to GAT at position 271, from GTG to GGC at position 274, from TAC to GTC at position 347, and from GCC to GTC at position 380.

Disclosed herein are aerobic hydrogen bacteria comprising one or more mutations in a nucleic acid sequence that encodes a mutated CbbR peptide.

Disclosed herein do aerobic hydrogen bacteria comprise a genetic modification, wherein the genetic modification comprises one or more mutations in a gene encoding a CbbR peptide. In an aspect, the mutated CbbR peptide is constitutively active. In an aspect, the mutated CbbR peptide is more active than a wild-type CbbR peptide or a non-mutated CbbR peptide.

In an aspect, the disclosed aerobic hydrogen bacteria comprising a genetic modification, wherein the genetic modification comprises one or more mutations in a gene encoding a CbbR peptide, are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the disclosed aerobic hydrogen bacteria comprising a genetic modification, wherein the genetic modification comprises one or more mutations in a gene encoding a CbbR peptide, produce n-butanol when cultured in the presence of oxygen, hydrogen, and carbon dioxide and in the dark. In an aspect, the aerobic hydrogen bacteria are isolated.

In an aspect, the mutated CbbR peptide of the aerobic hydrogen bacteria is mutated. In an aspect, the mutated CbbR peptide of the aerobic hydrogen bacteria is mutated in such a way that it results in a codon change in the wild-type sequence. For example, disclosed herein are aerobic hydrogen bacteria comprising a codon change in SEQ ID NO: 1. In an aspect, the amino acid mutation is L79F. (SEQ ID NO: 2). In an aspect, the amino acid mutation is E87K. (SEQ ID NO: 3). In an aspect, the amino acid mutation is E87K/G242S. (SEQ ID NO: 4). In an aspect, the amino acid mutation is G98R. (SEQ ID NO: 5). In an aspect, the amino acid mutation is A117V. (SEQ ID NO: 6). In an aspect, the amino acid mutation is G125D. (SEQ ID NO: 7). In an aspect, the amino acid mutation is G125S/V265M. (SEQ ID NO: 8). In an aspect, the amino acid mutation is D144N. (SEQ ID NO: 9). In an aspect, the amino acid mutation is D148N. (SEQ ID NO: 10). In an aspect, the amino acid mutation is A167V. (SEQ ID NO: 11). In an aspect, the amino acid mutation is G205D. (SEQ ID NO: 12). In an aspect, the amino acid mutation is G205S. (SEQ ID NO: 23). In an aspect, the amino acid mutation is G205D/G118D. (SEQ ID NO: 13). In an aspect, the amino acid mutation is G205D/R283H. (SEQ ID NO: 14). In an aspect, the amino acid mutation is P221S. (SEQ ID NO: 15). In an aspect, the amino acid mutation is P221S/T299I. (SEQ ID NO: 16). In an aspect, the amino acid mutation is T232A. (SEQ ID NO: 17). In an aspect, the amino acid mutation is T232I. (SEQ ID NO: 18). In an aspect, the amino acid mutation is P269S. (SEQ ID NO: 19). In an aspect, the amino acid mutation is P269S/T299I. (SEQ ID NO: 20). In an aspect, the amino acid mutation is R272Q. (SEQ ID NO: 21). In an aspect, the amino acid mutation is G80D/S106N/G261E. (SEQ ID NO: 22). In an aspect, the mutated CbbR peptide comprises a combination of codon changes selected from the following: L79F, E87K, E87K/G242S, G98R, A117V, G125D, G125S/V265M, D144N, D148N, A167V, G205D, G205S, G205D/G118D, G205D/R283H, P221S, P221S/T299I, T232A, T232I, P269S, P269S/T299I, R272Q, and G80D/S106N/G261E.

Disclosed herein are recombinant aerobic hydrogen bacteria, comprising a knockout mutation in gene phaC1 or gene phaC2 (encoding the poly(3-hydroxybutyrate) polymerase enzyme), wherein the knockout mutation decreases the amount of peptide produced in the recombinant aerobic hydrogen bacteria when compared to an aerobic hydrogen bacteria lacking the knockout mutation grown under identical reaction conditions.

In an aspect, the construct for the phaC1 knockout comprises SEQ ID NO: 37.

In an aspect, the disclosed aerobic hydrogen bacteria comprising a knockout mutation in gene phaC1 or gene phaC2 are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

Disclosed herein are aerobic hydrogen bacteria, wherein one or more endogenous genes is silenced or knocked out.

Disclosed herein are aerobic hydrogen bacteria, wherein one or more endogenous genes is silenced or knocked out. In an aspect, the one or more genes encode a peptide capable of converting (i) acetyl-CoA to acetoacetyl-CoA, (ii) acetoacetyl-CoA to (3-hydroxybutyryl-CoA, or (iii) β-hydroxybutyryl-CoA to polyhydroxyalkanoate.

In an aspect, the disclosed aerobic hydrogen bacteria, wherein one or more endogenous genes is silenced or knocked out, are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the one or more endogenous genes that is knocked out or silenced is selected from the group consisting of phaA, phaB1, phaC1, or phaC2. In an aspect, the construct for the phaC1 knockout comprises SEQ ID NO: 37. In an aspect, the construct for the phaC1/phaA/phaB1 knockout comprises SEQ ID NO: 38.

Disclosed herein are aerobic hydrogen bacteria comprising (i) one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, (ii) a genetic modification, wherein the genetic modification comprises one or more mutations in a gene encoding a ribulose bisphosphate carboxylase peptide, and (iii) a genetic modification, wherein the genetic modification comprises one or more mutations in a gene encoding a CbbR peptide.

In an aspect, the disclosed aerobic hydrogen bacteria are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the mutated ribulose bisphosphate carboxylase peptide of the aerobic hydrogen bacteria comprises is mutated. In an aspect, the mutated ribulose bisphosphate carboxylase peptide of the aerobic hydrogen bacteria is mutated in such a way that it results in a codon change in the wild-type sequence. For example, disclosed herein are aerobic hydrogen bacteria comprising a codon change in SEQ ID NO: 24. In an aspect, the codon change is from GGC to GGT at position 264. In an aspect, the codon change is from TCG to ACC at position 265. In an aspect, the change is S265T (SEQ ID NO: 25). In an aspect, the codon change is from GAC to GAT at position 271. In an aspect, the codon change is from GTG to GGC at position 274. In an aspect, the change is V274G (SEQ ID NO: 26). In an aspect, the codon change is from TAC to GTC at position 347. In an aspect, the change is Y347V (SEQ ID NO: 27). In an aspect, the codon change is from GCC to GTC at position 380. In an aspect, the change is A380V (SEQ ID NO: 28). In an aspect, the mutated ribulose bisphosphate carboxylase peptide comprises a combination of codon changes selected from the following: from GGC to GGT at position 264, from TCG to ACC at position 265, from GAC to GAT at position 271, from GTG to GGC at position 274, from TAC to GTC at position 347, and from GCC to GTC at position 380.

In an aspect, the mutated CbbR peptide of the aerobic hydrogen bacteria is mutated. In an aspect, the mutated CbbR peptide of the aerobic hydrogen bacteria is mutated in such a way that it results in a codon change in the wild-type sequence. For example, disclosed herein are aerobic hydrogen bacteria comprising a codon change in SEQ ID NO: 1. In an aspect, the amino acid mutation is L79F. (SEQ ID NO: 2). In an aspect, the amino acid mutation is E87K. (SEQ ID NO: 3). In an aspect, the amino acid mutation is E87K/G242S. (SEQ ID NO: 4). In an aspect, the amino acid mutation is G98R. (SEQ ID NO: 5). In an aspect, the amino acid mutation is A117V. (SEQ ID NO: 6). In an aspect, the amino acid mutation is G125D. (SEQ ID NO: 7). In an aspect, the amino acid mutation is G125S/V265M. (SEQ ID NO: 8). In an aspect, the amino acid mutation is D144N. (SEQ ID NO: 9). In an aspect, the amino acid mutation is D148N. (SEQ ID NO: 10). In an aspect, the amino acid mutation is A167V. (SEQ ID NO: 11). In an aspect, the amino acid mutation is G205D. (SEQ ID NO: 12). In an aspect, the amino acid mutation is G205S. (SEQ ID NO: 23). In an aspect, the amino acid mutation is G205D/G118D. (SEQ ID NO: 13). In an aspect, the amino acid mutation is G205D/R283H. (SEQ ID NO: 14). In an aspect, the amino acid mutation is P221S. (SEQ ID NO: 15). In an aspect, the amino acid mutation is P221S/T299I. (SEQ ID NO: 16). In an aspect, the amino acid mutation is T232A. (SEQ ID NO: 17). In an aspect, the amino acid mutation is T232I. (SEQ ID NO: 18). In an aspect, the amino acid mutation is P269S. (SEQ ID NO: 19). In an aspect, the amino acid mutation is P269S/T299I. (SEQ ID NO: 20). In an aspect, the amino acid mutation is R272Q. (SEQ ID NO: 21). In an aspect, the amino acid mutation is G80D/S106N/G261E. (SEQ ID NO: 22). In an aspect, the mutated CbbR peptide comprises a combination of codon changes selected from the following: L79F, E87K, E87K/G242S, G98R, A117V, G125D, G125S/V265M, D144N, D148N, A167V, G205D, G205S, G205D/G118D, G205D/R283H, P221S, P221S/T299I, T232A, T232I, P269S, P269S/T299I, R272Q, and G80D/S106N/G261E.

In an aspect, the aerobic hydrogen disclosed herein further comprise one or more endogenous genes is silenced or knocked out. In an aspect, the one or more genes encode a peptide capable of converting (i) acetyl-CoA to acetoacetyl-CoA, (ii) acetoacetyl-CoA to β-hydroxybutyryl-CoA, or (iii) β-hydroxybutyryl-CoA to polyhydroxyalkanoate. In an aspect, the one or more endogenous gene that is knocked out or silenced is selected from the group consisting of phaA, phaB 1, phaC1, or phaC2. In an aspect, the construct for the phaC1 knockout comprises SEQ ID NO: 37. In an aspect, the construct for the phaC1/phaA/phaB1 knockout comprises SEQ ID NO: 38.

It is also understood that the disclosed compositions can be employed in one or more of the methods disclosed herein.

i) Genes

a. Exogenous

In an aspect, the genes disclosed herein are exogenous to an aerobic hydrogen bacteria such as, for example, Ralstonia eutropha.

(1) Ribulose Bisphosphate Carboxylase

In an aspect, ribulose bisphosphate carboxylase (RubisCO) can be identified by the gene symbol Rru_A2400. In an aspect, the Rru_A2400 gene is exogenous to one or more particular organisms. In an aspect, the Rru_A2400 gene is a Rhodospirillum rubrum gene and is identified by NCBI Gene ID No. 3835834. In an aspect, the Rhodospirillum rubrum Rru_A2400 gene comprises the nucleotide sequence identified by NCBI Accession No. NC_007643.1. In an aspect, the protein product of the R. rubrum Rru_A2400 gene has the Accession No. YP_427487. In an aspect, Rru_A2400 is referred to as wild-type RubisCO large-subunit gene (cbbM).

In an aspect, ribulose bisphosphate carboxylase (RubisCO) can be identified by the gene symbol rbcL. In an aspect, the rbcL gene is exogenous to one or more particular organisms. In an aspect, the rbcL gene is a Synechococcus elongatus gene and is identified by NCBI Gene ID No. 3200134. In an aspect, the Synechococcus elongatus rbcL gene comprises the nucleotide sequence identified by NCBI Accession No. NC_006576.1. In an aspect, the protein product of the S. elongatus rbcL gene has the Accession No. YP_170840. In an aspect, rbcL is referred to as the ribulose bisphosphate carboxylase large subunit.

In an aspect, ribulose bisphosphate carboxylase (RubisCO) can be identified by the gene symbol rbcS. In an aspect, the rbcS gene is exogenous to one or more particular organisms. In an aspect, the rbcS gene is a Synechococcus elongates gene and is identified by NCBI Gene ID No. 3200023. In an aspect, the Synechococcus elongatus rbcS gene comprises the nucleotide sequence identified by NCBI Accession No. NC_006576.1. In an aspect, the protein product of the S. elongates rbcS gene has the Accession No. YP_170839.1. In an aspect, rbcS is referred to as the ribulose bisphosphate carboxylase small subunit.

In an aspect, ribulose bisphosphate carboxylase (RubisCO) can be identified by the gene symbol rbcL. In an aspect, the rbcL gene is exogenous to one or more particular organisms. In an aspect, the rbcL gene is an Archaeoglobus fulgidus gene and is identified by NCBI Gene ID No. 1484861. In an aspect, the Archaeoglobus fulgidus rbcL gene comprises the nucleotide sequence identified by NCBI Accession No. NC_000917.1. In an aspect, the protein product of the A. fulgidus rbcL gene has the Accession No. NP_070466. In an aspect, rbcL is referred to as the ribulose bisphosphate carboxylase large subunit.

In an aspect, ribulose bisphosphate carboxylase (RubisCO) can be identified by the gene symbol rbcL. In an aspect, the rbcL gene is exogenous to one or more particular organisms. In an aspect, the rbcL gene is a Methanosarcina acetivorans gene and is identified by NCBI Gene ID No. 1476449. In an aspect, the Methanosarcina acetivorans rbcL gene comprises the nucleotide sequence identified by NCBI Accession No. NC_003552.1. In an aspect, the protein product of the M. acetivorans rbcL gene has the Accession No. NP_619414.1. In an aspect, rbcL is referred to as the ribulose bisphosphate carboxylase large subunit.

(2) Acetyl-CoA Acetyltransferase

In an aspect, acetyl-CoA acetyltransferase can be identified by the gene symbol atoB. In an aspect, the atoB gene is exogenous to one or more particular organisms. In an aspect, the atoB gene is an E. coli gene and is identified by NCBI Gene ID No. 946727. In an aspect, the E. coli atoB gene has the nucleotide sequence identified by NCBI Accession No. NC_000913.2.

In an aspect, acetyl-CoA acetyltransferase can be identified by the gene symbol thil. In an aspect, the thil gene is exogenous to one or more particular organisms. In an aspect, the thil gene is a Clostridium acetobutylicum gene and is identified by NCBI Gene ID No. 1116083. In an aspect, the C. acetobutylicum thil gene has the nucleotide sequence identified by NCBI Accession No. NC_001988.2.

The art is familiar with the methods and techniques used to identify other acetyl-CoA Acetyltransferase genes and nucleotide sequences.

(3) 3-Hydroxybutyryl-CoA Dehydratase

In an aspect, 3-hydroxybutyryl-CoA dehydratase can be identified by the gene symbol crt. In an aspect, the crt gene is exogenous to one or more particular organisms. In an aspect, the crt gene is a Clostridium acetobutylicum gene and is identified by NCBI Gene ID No. 1118895. In an aspect, the C. acetobutylicum crt gene has the nucleotide sequence identified by NCBI Accession No. NC_003030.1.

The art is familiar with the methods and techniques used to identify other 3-hydroxybutyryl-CoA dehydratase genes and nucleotide sequences.

(4) Butyryl-CoA Dehydrogenase

In an aspect, butyryl-CoA dehydrogenase can be identified by the gene symbol bcd. In an aspect, the bcd gene is exogenous to one or more particular organisms. In an aspect, the bcd gene is a Clostridium acetobutylicum gene and is identified by NCBI Gene ID No. 1118894. In an aspect, the C. acetobutylicum bcd gene has the nucleotide sequence identified by NCBI Accession No. NC_003030.1.

The art is familiar with the methods and techniques used to identify other butyryl-CoA dehydrogenase genes and nucleotide sequences.

(5) Butanol Dehydrogenase

In an aspect, butanol dehydrogenase is NADH-dependent. In an aspect, NADH-dependent butanol dehydrogenase can be identified by the gene symbol bdhA. In an aspect, the bdhA gene is exogenous to one or more particular organisms. In an aspect, the bdhA gene is a Clostridium acetobutylicum gene and is identified by NCBI Gene ID No. 1119481. In an aspect, the C. acetobutylicum bdhA gene has the nucleotide sequence identified by NCBI Accession No. NC_003030.1.

In an aspect, NADH-dependent butanol dehydrogenase identified by the gene symbol bdhB. In an aspect, the bdhB gene is exogenous to one or more particular organisms. In an aspect, the bdhB gene is a Clostridium acetobutylicum gene and is identified by NCBI Gene ID No. 1119480. In an aspect, the C. acetobutylicum bdhB gene has the nucleotide sequence identified by NCBI Accession No. NC_003030.1.

The art is familiar with the methods and techniques used to identify other butanol dehydrogenase genes and nucleotide sequences.

(6) Electron-Transferring Flavoprotein

In an aspect, electron-transferring flavoprotein large subunit can be identified by the gene symbol etfA. In an aspect, the eftA gene is exogenous to one or more particular organisms. In an aspect, the etfA gene is a Clostridium acetobutylicum gene and is identified by NCBI Gene ID No. 1118726. In a further aspect, the etfA gene is a Clostridium acetobutylicum gene and is identified by NCBI Gene ID No. 1118892. In an aspect, the C. acetobutylicum etfA gene has the nucleotide sequence identified by NCBI Accession No. NC_003030.1.

In an aspect, electron-transferring flavoprotein small subunit can be identified by the gene symbol etfB. In an aspect, the eftB gene is exogenous to one or more particular organisms. In an aspect, the etfB gene is a Clostridium acetobutylicum gene and is identified by NCBI Gene ID No. 1118727. In a further aspect, the etfB electron transfer flavoprotein subunit beta gene is a Clostridium acetobutylicum gene and is identified by NCBI Gene ID No. 1118893. In an aspect, the C. acetobutylicum etfA and the etfA(beta) genes have the nucleotide sequence identified by NCBI Accession No. NC_003030.1.

The art is familiar with the methods and techniques used to identify other electron-transferring flavoproteins (large and beta) genes and nucleotide sequences.

(7) 3-Hydroxybutyryl-CoA Dehydrogenase

In an aspect, 3-hydroxybutyryl-CoA dehydrogenase can be identified by the gene symbol hbd. In an aspect, the hbd gene is exogenous to one or more particular organisms. In an aspect, the hbd gene is a Clostridium acetobutylicum gene and is identified by NCBI Gene ID No. 1118891. In an aspect, the C. acetobutylicum hbd gene has the nucleotide sequence identified by NCBI Accession No. NC_003030.1.

The art is familiar with the methods and techniques used to identify other 3-hydroxybutyryl-CoA dehydrogenase genes and nucleotide sequences.

(8) Bifunctional Acetaldehyde-coA/Alcohol Dehydrogenase

In an aspect, bifunctional acetaldehyde-CoA/alcohol dehydrogenase can be identified by the gene symbol adhe 1. In an aspect, the adhe 1 gene is exogenous to one or more particular organisms. In an aspect, the adhe1 gene is a Clostridium acetobutylicum gene and is identified by NCBI Gene ID No. 1116167. In an aspect, the C. acetobutylicum adhe1 gene has the nucleotide sequence identified by NCBI Accession No. NC_001988.2.

In an aspect, bifunctional acetaldehyde-CoA/alcohol dehydrogenase can be identified by the gene symbol adhe2. In an aspect, the adhe2 gene is exogenous to one or more particular organisms. In an aspect, the adhe gene2 is a Clostridium acetobutylicum gene and is identified by NCBI Gene ID No. 1116040. In an aspect, the C. acetobutylicum adhe2 gene has the nucleotide sequence identified by NCBI Accession No. NC_001988.2.

The art is familiar with the methods and techniques used to identify other bifunctional acetaldehyde-CoA/alcohol dehydrogenase genes and nucleotide sequences.

(9) Acetaldehyde Dehydrogenase

In an aspect, acetaldehyde dehydrogenase is acetaldehyde-CoA dehydrogenase II (NAD-binding). In an aspect, acetaldehyde-CoA dehydrogenase II (NAD-binding) can be identified by the gene symbol mhpF. In an aspect, the mhpF gene is exogenous to one or more particular organisms. In an aspect, the mhpF is an Escherichia coli gene and is identified by NCBI Gene ID No. 945008. In an aspect, the E. coli mhpF gene has the nucleotide sequence identified by NCBI Accession No. NC_000913.2. In an aspect, the protein product of the E. coli mhpF gene has the Accession No. NP_414885.

The art is familiar with the methods and techniques used to identify other acetaldehyde-CoA dehydrogenase II genes and nucleotide sequences.

(10) Aldehyde Decarbonylase

In an aspect, aldehyde decarbonylase can be identified by the gene symbol Synpcc7942_1593. In an aspect, the Synpcc7942_1593 gene is exogenous to one or more particular organisms. In an aspect, the Synpcc7942_1593 is a Synechococcus elongatus gene and is identified by NCBI Gene ID No. 3775017. In an aspect, the Synechococcus elongatus Synpcc7942_1593 gene has the nucleotide sequence identified by NCBI Accession No. NC_007604.1 In an aspect, the protein product of the S. elongatus Synpcc7942_1593 gene has the Accession No. YP_400610.

The art is familiar with the methods and techniques used to identify other aldehyde decarbonylase genes and nucleotide sequences.

(11) Acyl-ACP Reductase

In an aspect, acyl-ACP reductase can be identified by the gene symbol Synpcc7942_1594. In an aspect, the Synpcc7942_1594 gene is exogenous to one or more particular organisms. In an aspect, the Synpcc7942_1594 is a Synechococcus elongatus gene and is identified by NCBI Gene ID No. 3775018. In an aspect, the Synechococcus elongatus Synpcc7942_1594 gene has the nucleotide sequence identified by NCBI Accession No. NC_007604.1. In an aspect, the protein product of the S. elongatus Synpcc7942_1594 gene has the Accession No. YP_400611.

The art is familiar with the methods and techniques used to identify other acyl-ACP reductase genes and nucleotide sequences.

(12) L-1,2-Propanediol Oxidoreductase

In an aspect, L-1,2-propanediol oxidoreductase can be identified by the gene symbol fucO. In an aspect, the fucO gene is exogenous to one or more particular organisms. In an aspect, the fucO is an Escherichia coli gene and is identified by NCBI Gene ID No. 947273. In an aspect, the E. coli fucO gene has the nucleotide sequence identified by NCBI Accession No. NC_000913.2. In an aspect, the protein product of the E. coli fucO gene has the Accession No. NP_417279. The art is familiar with the methods and techniques used to identify other L-1,2-propanediol oxidoreductase genes and nucleotide sequences.

(13) Acyltransferase

In an aspect, acyltransferase can be identified by the gene symbol yqeF. In an aspect, the yqeF gene is exogenous to one or more particular organisms. In an aspect, the yqeF is an Escherichia coli gene and is identified by NCBI Gene ID No. 947324. In an aspect, the E. coli yqeF gene has the nucleotide sequence identified by NCBI Accession No. NC_000913.2.

The art is familiar with the methods and techniques used to identify other acyltransferase genes and nucleotide sequences.

(14) 3-Oxoacyl-ACP Synthase

In an aspect, 3-oxoacyl-ACP synthase can be identified by the gene symbol Sama_1182. In an aspect, the Sama_1182 gene is exogenous to one or more particular organisms. In an aspect, the Sama_1182 gene is a Shewanella amazonensis gene and is identified by NCBI Gene ID No. 4603434. In an aspect, the Shewanella amazonensis Sama_1182 gene has the nucleotide sequence identified by NCBI Accession No. NC_008700.1. In an aspect, the protein product of the S. amazonensis Sama_1182 gene has the Accession No. YP_927059.

In an aspect, 3-oxoacyl-ACP synthase can be identified by the gene symbol SO_1742. In an aspect, the SO_1742 gene is exogenous to one or more particular organisms. In an aspect, the SO_1742 gene is a Shewanella oneidensis gene and is identified by NCBI Gene ID No. 1169520. In an aspect, the Shewanella oneidensis SO_1742 gene has the nucleotide sequence identified by NCBI Accession No. NC_004347.1. In an aspect, the protein product of the S. oneidensis SO_1742 gene has the Accession No. NP_717352.1.

The art is familiar with the methods and techniques used to identify other 3-oxoacyl-ACP synthase genes and nucleotide sequences.

(15) Fused 3-Hydroxybutyryl-CoA Epimerase/Delta(3)-Cis-Delta(2)-Trans-Enoyl-CoA Isomerase/Enoyl-CoA Hydratase/3-Hydroxyacyl-CoA Dehydrogenase

In an aspect, fused 3-hydroxybutyryl-CoA epimerase/delta(3)-cis-delta(2)-trans-enoyl-CoA isomerase/enoyl-CoA hydratase/3-hydroxyacyl-CoA dehydrogenase can be identified by the gene symbol fadB. In an aspect, the fadB gene is exogenous to one or more particular organisms. In an aspect, the fadB is an Escherichia coli gene and is identified by NCBI Gene ID No. 948336. In an aspect, the E. coli fadB gene has the nucleotide sequence identified by NCBI Accession No. NC_000913.2.

The art is familiar with the methods and techniques used to identify other fused 3-hydroxybutyryl-CoA epimerase/delta(3)-cis-delta(2)-trans-enoyl-CoA isomerase/enoyl-CoA hydratase/3-hydroxyacyl-CoA dehydrogenase genes and nucleotide sequences.

(16) Short Chain Dehydrogenase

In an aspect, short chain dehydrogenase can be identified by the gene symbol Maqu_2507 or Ma2507. In an aspect, the Ma2507 gene is exogenous to one or more particular organisms. In an aspect, the Ma2507 gene is a Marinobacter aquaeolei gene and is identified by NCBI Gene ID No. 4655706. In an aspect, the Marinobacter aquaeolei Ma2507 gene has the nucleotide sequence identified by NCBI Accession No. NC_008740.1. In an aspect, the protein product of the M. aquaeolei gene has the Accession No. YP_959769.

The art is familiar with the methods and techniques used to identify other short chain dehydrogenase genes and nucleotide sequences.

(17) Trans-2-Enoyl-CoA Reductase

In an aspect, trans-2-enoyl-CoA reductase can be identified by the gene symbol TDE0597 or ter. In an aspect, the ter gene is exogenous to one or more particular organisms. In an aspect, the ter gene is a Treponema denticola gene and is identified by NCBI Gene ID No. 2741560. In an aspect, the T. denticola ter gene has the nucleotide sequence identified by NCBI Accession No. NC_002967.9.

The art is familiar with the methods and techniques used to identify other trans-2-enoyl-CoA reductase genes and nucleotide sequences.

(18) Others

In an aspect, a hypothetical protein can be identified by the gene symbol syc0051_d. In an aspect, the syc0051_d gene is exogenous to one or more particular organisms. In an aspect, the syc0051_d gene is a Synechococcus elongatus gene and is identified by NCBI Gene ID No. 3200246. In an aspect, the Synechococcus elongatus syc0051_d gene has the nucleotide sequence identified by NCBI Accession No. NC_006576.1. In an aspect, the protein product of the Synechococcus elongatus syc0051_d gene has the Accession No. YP_170761.

In an aspect, a hypothetical protein can be identified by the gene symbol syc0050_d. In an aspect, the syc0050_d gene is exogenous to one or more particular organisms. In an aspect, the syc0050_d gene is a Synechococcus elongatus gene and is identified by NCBI Gene ID No. 3200028. In an aspect, the Synechococcus elongatus syc0050_d gene has the nucleotide sequence identified by NCBI Accession No. NC_006576.1. In an aspect, the protein product of the Synechococcus elongatus syc0050_d gene has the Accession No. YP_170760.

In an aspect, a hypothetical protein can be identified by the gene symbol alr5284. In an aspect, the alr5284 gene is exogenous to one or more particular organisms. In an aspect, the alr5284 gene is a Nostoc sp. gene and is identified by NCBI Gene ID No. 1108888. In an aspect, the Nostoc sp. alr5284 gene has the nucleotide sequence identified by NCBI Accession No. NC_003272.1. In an aspect, the protein product of the Nostoc sp. alr5284 gene has the Accession No. NP_489324.1.

In an aspect, a hypothetical protein can be identified by the gene symbol alr5283. In an aspect, the alr5283 gene is exogenous to one or more particular organisms. In an aspect, the alr5283 gene is a Nostoc sp. gene and is identified by NCBI Gene ID No. 1108887. In an aspect, the Nostoc sp. alr5283 gene has the nucleotide sequence identified by NCBI Accession No. NC_003272.1. In an aspect, the protein product of the Nostoc sp. alr5283 gene has the Accession No. NP_489323.1.

In an aspect, a hypothetical protein can be identified by the gene symbol sll0209. In an aspect, the sll0209 gene is exogenous to one or more particular organisms. In an aspect, the sll0209 gene is a Synechocystis sp. gene and is identified by NCBI Gene ID No. 952637. In an aspect, the Synechocystis sp. sll0209 gene has the nucleotide sequence identified by NCBI Accession No. NC_000911.1. In an aspect, the protein product of the Nostoc sp. sll0209 gene has the Accession No. NP_442146.

In an aspect, a hypothetical protein can be identified by the gene symbol sll0208. In an aspect, the sll0208 gene is exogenous to one or more particular organisms. In an aspect, the sll0208 gene is a Synechocystis sp. gene and is identified by NCBI Gene ID No. 952286. In an aspect, the Synechocystis sp. sll0208 gene has the nucleotide sequence identified by NCBI Accession No. NC_000911.1. In an aspect, the protein product of the Nostoc sp. sll0208 gene has the Accession No. NP_442147.

b. Endogenous

In an aspect, the genes disclosed herein are endogenous to an aerobic hydrogen bacteria such as, for example, genes of Ralstonia eutropha.

(1) Transcription Regulator LysR

In an aspect, transcription regulator LysR can be identified by the gene symbol cbbR. In an aspect, the cbbR gene is endogenous to one or more particular organisms. In an aspect, the cbbR gene is a Ralstonia eutropha gene and is identified by NCBI Gene ID No. 4456355. In an aspect, the R. eutropha cbbR gene has the nucleotide sequence identified by NCBI Accession No. NC_008314.1. In an aspect, the protein product of the R. eutropha cbbR gene has the Accession No. YP_840915. The art is familiar with the methods and techniques used to identify other transcription regulator LysR genes and nucleotide sequences.

(2) Ribulose Bisphosphate Carboxylase

In an aspect, ribulose bisphosphate carboxylase (RubisCO) can be identified by the gene symbol rbcL. In an aspect, the rbcL gene is endogenous to one or more particular organisms. In an aspect, the rbcL gene is a Ralstonia eutropha gene and is identified by NCBI Gene ID No. 4456354. In an aspect, the R. eutropha rbcL gene comprises the nucleotide sequence identified by NCBI Accession No. NC_008314.1. In an aspect, the protein product of the E. coli fucO gene has the Accession No. YP_840914. In an aspect, rbcL is referred to as the genomic RubisCO large-subunit.

In an aspect, ribulose bisphosphate carboxylase (RubisCO) can be identified by the gene symbol cbbS2. In an aspect, the cbbS2 gene is endogenous to one or more particular organisms. In an aspect, the cbbS2 gene is a Ralstonia eutropha gene and is identified by NCBI Gene ID No. 4456353. In an aspect, the R. eutropha cbbS2 gene comprises the nucleotide sequence identified by NCBI Accession No. NC_008314.1. In an aspect, the protein product of the R. eutropha cbbS2 gene has the Accession No. YP_840913. In an aspect, cbbS2 is referred to as the genomic RubisCO small-subunit.

In an aspect, ribulose bisphosphate carboxylase (RubisCO) can be identified by the gene symbol rbcL. In an aspect, the rbcL gene is endogenous to one or more particular organisms. In an aspect, the rbcL gene is a Ralstonia eutropha gene and is identified by NCBI Gene ID No. 2656546. In an aspect, the R. eutropha rbcL gene comprises the nucleotide sequence identified by NCBI Accession No. NC_005241.1. In an aspect, the protein product of the R. eutropha rbcL gene has the Accession No. NP_943062. In an aspect, rbcL is referred to as the megaplasmid RubisCO large-subunit.

In an aspect, ribulose bisphosphate carboxylase (RubisCO) can be identified by the gene symbol cbbSp. In an aspect, the cbbSp gene is endogenous to one or more particular organisms. In an aspect, the cbbSp gene is a Ralstonia eutropha gene and is identified by NCBI Gene ID No. 2656545. In an aspect, the R. eutropha cbbSp gene comprises the nucleotide sequence identified by NCBI Accession No. NC_005241.1. In an aspect, the protein product of the R. eutropha cbbSp gene has the Accession No. NP_943061. In an aspect, cbbSp is referred to as the megaplasmid RubisCO small-subunit.

The art is familiar with the methods and techniques used to identify other ribulose bisphosphate carboxylase genes and nucleotide sequences.

(3) Acetyl-CoA Acetyltransferase

In an aspect, acetyl-CoA acetyltransferase can be identified by the gene symbol phaA. In an aspect, the phaA gene is endogenous to one or more particular organisms. In an aspect, the phaA gene is a Ralstonia eutropha gene and is identified by NCBI Gene ID No. 4249783. In an aspect, the R. eutropha phaA gene has the nucleotide sequence identified by NCBI Accession No. NC_008313.1.

The art is familiar with the methods and techniques used to identify other acetyl-CoA acetyltransferase genes and nucleotide sequences.

(4) Acetyacetyl-CoA Reductase

In an aspect, acetyacetyl-CoA reductase can be identified by the gene symbol phaB 1. In an aspect, the phaB 1 gene is endogenous to one or more particular organisms. In an aspect, the phaA gene is a Ralstonia eutropha gene and is identified by NCBI Gene ID No. 4249784. In an aspect, the R. eutropha phaB 1 gene has the nucleotide sequence identified by NCBI Accession No. NC_008313.1.

The art is familiar with the methods and techniques used to identify other acetyacetyl-CoA reductase genes and nucleotide sequences.

(5) Poly(3-Hydroxybutyrate)Polymerase

In an aspect, poly(3-hydroxybutyrate) polymerase can be identified by the gene symbol phaC 1. In an aspect, the phaC1 gene is endogenous to one or more particular organisms. In an aspect, the phaC1 gene is a Ralstonia eutropha gene and is identified by NCBI Gene ID No. 4250156. In an aspect, the R. eutropha phaC1 gene has the nucleotide sequence identified by NCBI Accession No. NC_008313.1. The art is familiar with the methods and techniques used to identify other poly(3-hydroxybutyrate) polymerase genes and nucleotide sequences.

In an aspect, poly(3-hydroxybutyrate) polymerase can be identified by the gene symbol phaC2. In an aspect, the phaC2 gene is endogenous to one or more particular organisms. In an aspect, the phaC2 gene is a Ralstonia eutropha gene and is identified by NCBI Gene ID No. 4250157. In an aspect, the R. eutropha phaC2 gene has the nucleotide sequence identified by NCBI Accession No. NC_008313.1.

The art is familiar with the methods and techniques used to identify other poly(3-hydroxybutyrate) polymerase genes and nucleotide sequences.

(6) NAD(P) Transhydrogenase

In an aspect, NAD(P) transhydrogenase (subunit alpha) can be identified by the gene symbol pntAa3. In an aspect, the pntAa3 gene is endogenous to one or more particular organisms. In an aspect, the pntAa3 gene is a Ralstonia eutropha gene and is identified by NCBI Gene ID No. 4250035. In an aspect, the R. eutropha pntAa3 gene has the nucleotide sequence identified by NCBI Accession No. NC_008313.1.

The art is familiar with the methods and techniques used to identify other NAD(P) transhydrogenase genes and nucleotide sequences.

(7) NADH:Flavin Oxidoreductase/NADH Oxidase

In an aspect, NADH:flavin oxidoreductase/NADH oxidase family protein can be identified by the gene symbol H16_B1142. In an aspect, the H16_B1142 gene is endogenous to one or more particular organisms. In an aspect, the H16_B1142 gene is a Ralstonia eutropha gene and is identified by NCBI Gene ID No. 4455963. In an aspect, the R. eutropha H16_B1142 gene has the nucleotide sequence identified by NCBI Accession No. NC_008314.1.

The art is familiar with the methods and techniques used to identify other NADH:flavin oxidoreductase/NADH oxidase genes and nucleotide sequences.

(8) Alcohol Dehydrogenase

In an aspect, alcohol dehydrogenase can be identified by the gene symbol H16_A3330. In an aspect, the H16_A3330 gene is endogenous to one or more particular organisms. In an aspect, the H16_A3330 gene is a Ralstonia eutropha gene and is identified by NCBI Gene ID No. 4248484. In an aspect, the R. eutropha H16_A3330 gene has the nucleotide sequence identified by NCBI Accession No. NC_008313.1.

In an aspect, alcohol dehydrogenase can be identified by the gene symbol h16_A0861. In an aspect, the h16_A0861 gene is exogenous to one or more particular organisms. In an aspect, the h16_A0861 is a Ralstonia eutropha gene and is identified by NCBI Gene ID No. 4247415. In an aspect, the R. eutropha h16_A0861 gene has the nucleotide sequence identified by NCBI Accession No. NC_008313.1. In an aspect, the protein product of the R. eutropha h16_A0861 gene has the Accession No. YP_725376.

The art is familiar with the methods and techniques used to identify other alcohol dehydrogenase genes and nucleotide sequences.

(9) D-Beta-D-Heptose 7-Phophosphate Kinase

In an aspect, D-beta-D-heptose 7-phophosphate kinase can be identified by the gene symbol hldA. In an aspect, the hldA gene is endogenous to one or more particular organisms. In an aspect, the hldA gene is a Ralstonia eutropha gene and is identified by NCBI Gene ID No. 4250454. In an aspect, the R. eutropha hldA gene has the nucleotide sequence identified by NCBI Accession No. NC_008313.1.

The art is familiar with the methods and techniques used to identify other D-beta-D-heptose 7-phophosphate kinase genes and nucleotide sequences.

(10) Phosphate Acetyltransferase

In an aspect, phosphate acetyltransferase can be identified by the gene symbol ptal. In an aspect, the ptal gene is endogenous to one or more particular organisms. In an aspect, the ptal gene is a Ralstonia eutropha gene and is identified by NCBI Gene ID No. 4456117. In an aspect, the R. eutropha ptal gene has the nucleotide sequence identified by NCBI Accession No. NC_008314.1. In an aspect, the protein product from this gene is identified by Accession No. YP_841146.

The art is familiar with the methods and techniques used to identify other phosphate acetyltransferase genes and nucleotide sequences.

(11) Acetaldehyde Dehydrogenase

In an aspect, acetaldehyde dehydrogenase can be identified by the gene symbol mhpF. In an aspect, the mhpF gene is exogenous to one or more particular organisms. In an aspect, the mhpF is a R. eutropha gene and is identified by NCBI Gene ID No. 4456316. In an aspect, the R. eutropha mhpF gene has the nucleotide sequence identified by NCBI Accession No. NC_008314.1. In an aspect, the protein product of the R. eutropha mhpF gene has the Accession No. YP_728713.

In an aspect, acetaldehyde dehydrogenase can be identified by the gene symbol H16_B0596. In an aspect, the H16_B0596 gene is exogenous to one or more particular organisms. In an aspect, the H16_B0596 is a R. eutropha gene and is identified by NCBI Gene ID No. 4456557. In an aspect, the R. eutropha H16_B0596 gene has the nucleotide sequence identified by NCBI Accession No. NC_008314.1. In an aspect, the protein product of the R. eutropha mhpF gene has the Accession No. YP_728758.

The art is familiar with the methods and techniques used to identify other acetaldehyde dehydrogenase genes and nucleotide sequences.

(12) Acetate Kinase

In an aspect, acetate kinase can be identified by the gene symbol ackA. In an aspect, the ackA gene is endogenous to one or more particular organisms. In an aspect, the ptal gene is a Ralstonia eutropha gene and is identified by NCBI Gene ID No. 4456116. In an aspect, the R. eutropha ackA gene has the nucleotide sequence identified by NCBI Accession No. NC_008314.1. In an aspect, the protein product from this gene is identified by Accession No. YP_841145.

The art is familiar with the methods and techniques used to identify other acetate kinase genes and nucleotide sequences.

ii) Vectors

Disclosed herein are vectors comprising the disclosed compositions. Disclosed herein are vectors for use in the disclosed method. For example, one or more of the vectors disclosed herein can be used to transfect an aerobic hydrogen bacteria, a microbial organism or a microorganism. Also disclosed herein are aerobic hydrogen bacteria, microbial organisms and microorganisms transfected with or comprising one or more of the vectors described herein. For example, disclosed herein are E. coli comprising one or more of the vectors described herein. Also disclosed herein are aerobic hydrogen bacteria comprising one or more of the vectors described herein.

Disclosed herein is a vector comprising one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof.

In an aspect, the disclosed vector comprises one or more mutations in a nucleic acid sequence that encodes a mutated ribulose bisphosphate carboxylase peptide. In an aspect, the disclosed vector comprises one or more mutations in a nucleic acid sequence that encodes a mutated ribulose bisphosphate carboxylase peptide. In an aspect, the mutated ribulose bisphosphate carboxylase peptide of the aerobic hydrogen bacteria is mutated in such a way that it results in a codon change in the wild-type sequence. For example, disclosed herein are aerobic hydrogen bacteria comprising a codon change in SEQ ID NO: 24. In an aspect, the codon change is from GGC to GGT at position 264. In an aspect, the codon change is from TCG to ACC at position 265. In an aspect, the change is S265T (SEQ ID NO: 25). In an aspect, the codon change is from GAC to GAT at position 271. In an aspect, the codon change is from GTG to GGC at position 274. In an aspect, the change is V274G (SEQ ID NO: 26). In an aspect, the codon change is from TAC to GTC at position 347. In an aspect, the change is Y347V (SEQ ID NO: 27). In an aspect, the codon change is from GCC to GTC at position 380. In an aspect, the change is A380V (SEQ ID NO: 28). In an aspect, the mutated ribulose bisphosphate carboxylase peptide comprises a combination of codon changes selected from the following: from GGC to GGT at position 264, from TCG to ACC at position 265, from GAC to GAT at position 271, from GTG to GGC at position 274, from TAC to GTC at position 347, and from GCC to GTC at position 380.

In an aspect, the disclosed vector comprises one or more mutations in a nucleic acid sequence that encodes a mutated CbbR peptide. In an aspect, the disclosed vector comprises at least one nucleic acid molecule comprising a genetic modification, wherein the genetic modification comprises one or more mutations in a gene encoding a CbbR peptide. In an aspect, the mutated CbbR peptide of the aerobic hydrogen bacteria is mutated in such a way that it results in a codon change in the wild-type sequence. For example, disclosed herein are aerobic hydrogen bacteria comprising a codon change in SEQ ID NO: 1. In an aspect, the amino acid mutation is L79F. (SEQ ID NO: 2). In an aspect, the amino acid mutation is E87K. (SEQ ID NO: 3). In an aspect, the amino acid mutation is E87K/G242S. (SEQ ID NO: 4). In an aspect, the amino acid mutation is G98R. (SEQ ID NO: 5). In an aspect, the amino acid mutation is A117V. (SEQ ID NO: 6). In an aspect, the amino acid mutation is G125D. (SEQ ID NO: 7). In an aspect, the amino acid mutation is G125S/V265M. (SEQ ID NO: 8). In an aspect, the amino acid mutation is D144N. (SEQ ID NO: 9). In an aspect, the amino acid mutation is D148N. (SEQ ID NO: 10). In an aspect, the amino acid mutation is A167V. (SEQ ID NO: 11). In an aspect, the amino acid mutation is G205D. (SEQ ID NO: 12). In an aspect, the amino acid mutation is G205S. (SEQ ID NO: 23). In an aspect, the amino acid mutation is G205D/G118D. (SEQ ID NO: 13). In an aspect, the amino acid mutation is G205D/R283H. (SEQ ID NO: 14). In an aspect, the amino acid mutation is P221S. (SEQ ID NO: 15). In an aspect, the amino acid mutation is P221S/T299I. (SEQ ID NO: 16). In an aspect, the amino acid mutation is T232A. (SEQ ID NO: 17). In an aspect, the amino acid mutation is T232I. (SEQ ID NO: 18). In an aspect, the amino acid mutation is P269S. (SEQ ID NO: 19). In an aspect, the amino acid mutation is P269S/T299I. (SEQ ID NO: 20). In an aspect, the amino acid mutation is R272Q. (SEQ ID NO: 21). In an aspect, the amino acid mutation is G80D/S106N/G261E. (SEQ ID NO: 22). In an aspect, the mutated CbbR peptide comprises a combination of codon changes selected from the following: L79F, E87K, E87K/G242S, G98R, A117V, G125D, G125S/V265M, D144N, D148N, A167V, G205D, G205S, G205D/G118D, G205D/R283H, P221S, P221S/T299I, T232A, T232I, P269S, P269S/T299I, R272Q, and G80D/S106N/G261E.

In an aspect, the expression of the one or more exogenous nucleic acid molecules encoding a naturally encoding polypeptide of the disclosed vectors increases the efficiency of producing n-butanol.

In an aspect, the disclosed vector comprises crt, bcd, eftA, eftB, hbd, and adhE2 In an aspect, the disclosed vector comprises atoB, hbd, crt, ter, and adhE2. In an aspect, the disclosed vector comprises atoB, hbd, crt, ter, mhpF, and fucO. In an aspect, the disclosed vector comprises hbd, crt, ter, mhpF, fucO, and yqeF. In an aspect, the disclosed vector comprises atoB, hbd, crt, ter, and Ma2507. In an aspect, the disclosed vector comprises atoB, crt, ter, adheE2, and fadB.

In an aspect, the one or more exogenous nucleic acid molecules in the vectors is operably linked to a control element. In an aspect, the control element is a promoter. In an aspect, the promoter is constitutively active, or inducibly active, or tissue-specific, or development stage-specific. In an aspect, the promoter is cbbL (native), cbbL (constitutive), lac, tac, pha, cbbM, pBAD, or pseudomonas syringae. In an aspect, the cbbL (native) promoter is a R. eutropha promoter. In an aspect, the cbbL (native) promoter comprises SEQ ID NO: 29. In an aspect, the cbbL (constitutive) is a R. eutropha promoter. In an aspect, the cbbL (constitutive) promoter comprises SEQ ID NO: 30. In an aspect, the lac promoter is an E. coli promoter. In an aspect, the lac promoter comprises SEQ ID NO: 31. In an aspect, the tac promoter is a synthetic promoter. In an aspect, the tac promoter is an E. coli promoter. In an aspect, the tac promoter comprises SEQ ID NO: 32. In an aspect, the pha promoter is a R. eutropha promoter. In an aspect, the pha promoter comprises SEQ ID NO: 33. In an aspect, the cbbM promoter is a Rhodosporilium rubrum promoter. In an aspect, the cbbM promoter comprises SEQ ID NO: 34. In an aspect, the pBAD promoter is an arabinose inducible promoter. In an aspect, the pBAD promoter comprises SEQ ID NO: 35.

In an aspect, the vectors further comprise one or more optimized ribosome binding sites.

Disclosed herein are vectors p42 (SEQ ID NO: 45), p52 (SEQ ID NO: 46), p61 (SEQ ID NO: 40), p90 (SEQ ID NO:41), p91 (SEQ ID NO: 42), pBBR1MCS3-ptac (SEQ ID NO: 43), pBBR1MCS3-ptac (SEQ ID NO: 43), pBBR1MCS3-pBAD (SEQ ID NO: 44), pIND4 (Accession No. FM164773), CbbR reporter strain pVKcBBR, pHG1 (see J. Molecular Biology, 332: 369-383 (2003), pJQ-mUTR and pJQ-gUTR (see Gene, 127(1): 15-21 (1993)). Disclosed herein are vectors are illustrated in the Figures provided herein.

The vectors can be viral vectors and the viral vectors can optionally be self-inactivating. Furthermore, the expression of the one or more of the nucleic acid sequences of the vectors can be regulatable.

Also disclosed are cells and cell lines that comprise the vectors disclosed herein.

Also disclosed are vectors optionally comprising RNA export elements. The term “RNA export element” refers to a cis-acting post-transcriptional regulatory element that regulates the transport of an RNA transcript from the nucleus to the cytoplasm of a cell. Examples of RNA export elements include, but are not limited to, the human immunodeficiency virus (HIV) rev response element (RRE) (see e.g., Cullen et al. (1991) J. Virol. 65: 1053; and Cullen et al. (1991) Cell 58: 423-426), and the hepatitis B virus post-transcriptional regulatory element (PRE) (see e.g., Huang et al. (1995) Molec. and Cell. Biol. 15(7): 3864-3869; Huang et al. (1994) J. Virol. 68(5): 3193-3199; Huang et al. (1993) Molec. and Cell. Biol 13(12): 7476-7486), and U.S. Pat. No. 5,744,326. These references are incorporated herein by reference in their entirety for their teachings of RNA export elements). Generally, the RNA export element is placed within the 3′ UTR of a gene, and can be inserted as one or multiple copies. RNA export elements can be inserted into any or all of the separate vectors described herein.

Also disclosed are Internal Ribosome Entry Sites (IRES) and Internal Ribosome Entry Site-Like elements. Internal Ribosome Entry Sites (IRES) are cis-acting RNA sequences able to mediate internal entry of the 40S ribosomal subunit on some eukaryotic and viral messenger RNAs upstream of a translation initiation codon. Although sequences of IRESs are very diverse and are present in a growing list of mRNAs, IRES elements contain a conserved Yn-Xm-AUG unit (Y, pyrimidine; X, nucleotide), which appears essential for IRES function. Novel IRES sequences continue to be added to public databases every year and the list of unknown IRES sequences is certainly still very large.

IRES-like elements are also cis-acting sequences able to mediate internal entry of the 40S ribosomal subunit on some eukaryotic and viral messenger RNAs upstream of a translation initiation codon. Unlike IRES elements, in IRES-like elements, the Yn-Xm-AUG unit (Y, pyrimidine; X, nucleotide), which appears essential for IRES function, is not required.

The IRES or IRES-like element can be naturally occurring or non-naturally occurring. Examples of IRESs include, but are not limited to the IRES present in the IRES database at http://ifr31w3.toulouse.inserm.fr/IRESdatabase/. Examples of IRES can also include, but are not limited to, the EMC-virus IRES, or HCV-virus IRES. In addition, the IRES or IRES-like element can be mutated, wherein the function of the IRES or IRES-like element is retained.

Also disclosed are transcriptional control elements (TCEs). TCEs are elements capable of driving expression of nucleic acid sequences operably linked to them. The constructs disclosed herein comprise at least one TCE. TCEs can optionally be constitutive or regulatable.

Regulatable TCEs can comprise a nucleic acid sequence capable of being bound to a binding domain of a fusion protein expressed from a regulator construct such that the transcription repression domain acts to repress transcription of a nucleic acid sequence contained within the regulatable TCE.

Regulatable TCEs can be regulatable by, for example, tetracycline or doxycycline. Furthermore, the TCEs can optionally comprise at least one tet operator sequence. In one example, at least one tet operator sequence can be operably linked to a TATA box.

Furthermore, the TCE can be a promoter, as described elsewhere herein. Examples of promoters useful with vectors disclosed herein are given throughout the specification and examples. For example, promoters can include, but are not limited to, CMV based, CAG, SV40 based, heat shock protein, a mH1, a hH1, chicken β-actin, U6, Ubiquitin C, or EF-1α promoters.

Additionally, the TCEs disclosed herein can comprise one or more promoters operably linked to one another, portions of promoters, or portions of promoters operably linked to each other. For example, a transcriptional control element can include, but are not limited to a 3′ portion of a CMV promoter, a 5′ portion of a CMV promoter, a portion of the β-actin promoter, or a 3′CMV promoter operably linked to a CAG promoter.

“Enhancer” generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Each of the cited references is incorporated herein by reference in their entirety for their teachings of enhancers. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). Each of the cited references is incorporated herein by reference in their entirety for their teachings of potential locations of enhancers. They are usually between 10 and 300 bp in length, and they function in cis. Enhancers function to increase transcription from nearby promoters Enhancers also often contain response elements that mediate the regulation of transcription. Promoters can also contain response elements that mediate the regulation of transcription Enhancers often determine the regulation of expression of a gene.

The promoter and/or enhancer can be specifically activated either by light or specific chemical events which trigger their function. Systems can be regulated by reagents such as tetracycline and dexamethasone.

In some aspects, the promoter and/or enhancer region can act as a constitutive promoter and/or enhancer to maximize expression of the region of the transcription unit to be transcribed. In certain vectors the promoter and/or enhancer region are active in all cell types, even if it is only expressed in a particular type of cell at a particular time.

Also disclosed are cell lines comprising the vectors disclosed herein. Methods for producing cell lines are also described elsewhere herein.

The vectors described above and below are useful with any of the compositions and methods disclosed herein.

iii) Cultures

Disclosed herein are cultures of the disclosed aerobic hydrogen bacteria, microbial organism, and microorganisms.

The aerobic hydrogen bacteria, microbial organism, and microorganisms described herein can be cultured in a medium suitable for propagation of the microorganism, for example, NB medium.

Disclosed herein are culture conditions suitable for culture aerobic hydrogen bacteria, such as R. eutropha. (See, e.g., Tables 13 and 14 in Example 6). In an aspect, the aerobic hydrogen bacteria can be cultured in TSB as a medium at 100% air gas mix. In an aspect, aerobic hydrogen bacteria can be cultured in MOPS-Repaske's as a medium at 100% air gas mix. In an aspect, aerobic hydrogen bacteria can be cultured in MOPS-Repaske's as a medium at 33.3% H₂, 33.3% CO₂, 33.3% air gas mix. In an aspect, aerobic hydrogen bacteria can be cultured in MOPS-Repaske's as a medium at 5% H₂, 25% CO₂, 70% air.

Disclosed herein are culture conditions include aerobic or substantially aerobic growth or maintenance conditions. Exemplary aerobic conditions have been described previously and are well known in the art. Any of these conditions can be employed with the aerobic hydrogen bacteria of the present invention (e.g., R. eutropha or R. caspsulatus) as well as other aerobic conditions well known in the art. The culture conditions can include, for example, liquid culture procedures as well as fermentation and other large scale culture procedures. As described herein, yields of the biosynthetic products of the invention, such as n-butanol, can be obtained under aerobic or substantially aerobic culture conditions.

As described herein, one exemplary growth condition for achieving biosynthesis of n-butanol includes aerobic culture or fermentation conditions. In certain embodiments, the aerobic hydrogen bacteria of the invention can be sustained, cultured, or fermented under aerobic or substantially aerobic conditions. Briefly, aerobic conditions refer to an environment in the presence of oxygen.

The culture conditions described herein can be scaled up and grown continuously for manufacturing of n-butanol. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of n-butanol. Generally, and as with non-continuous culture procedures, the continuous and/or near-continuous production of n-butanol will include culturing a non-naturally occurring n-butanol producing organism of the invention in sufficient nutrients and medium to sustain and/or nearly sustain growth in an exponential phase. Continuous culture under such conditions can be include, for example, 1 day, 2, 3, 4, 5, 6 or 7 days or more. Additionally, continuous culture can include 1 week, 2, 3, 4 or 5 or more weeks and up to several months. Alternatively, the disclosed aerobic hydrogen bacteria of the invention can be cultured for hours, if suitable for a particular application. It is to be understood that the continuous and/or near-continuous culture conditions also can include all time intervals in between these exemplary periods. It is further understood that the time of culturing the aerobic hydrogen bacteria disclosed herein for a sufficient period of time to produce a sufficient amount of product for a desired purpose.

Fermentation procedures are well known in the art. Briefly, fermentation for the biosynthetic production of n-butanol can be utilized in, for example, fed-batch fermentation and batch separation; fed-batch fermentation and continuous separation, or continuous fermentation and continuous separation. Examples of batch and continuous fermentation procedures are well known in the art.

### C. Methods of Using the Compositions

Disclosed herein is a method of preparing n-butanol, the method comprising culturing engineered aerobic hydrogen in the dark and in a medium comprising oxygen, hydrogen, and carbon dioxide, and isolating the n-butanol.

Disclosed herein is a method of producing n-butanol, comprising (a) culturing a population of aerobic hydrogen bacteria autotrophically, wherein (i) the aerobic hydrogen bacteria comprise one or more exogenous nucleic acid molecules encoding a naturally occurring polypeptide, (ii) the carbon source comprises CO₂, and (b) recovering the n-butanol from the medium.

In an aspect, the aerobic hydrogen bacteria of the disclosed methods are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the one or more exogenous nucleic acid molecules encoding a naturally occurring polypeptide comprise ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof.

In an aspect, the aerobic hydrogen bacteria of the disclosed method comprise crt, bcd, eftA, eftB, hbd, and adhE2 In an aspect, the disclosed aerobic hydrogen bacteria comprise atoB, hbd, crt, ter, and adhE2. In an aspect, the disclosed aerobic hydrogen bacteria comprise atoB, hbd, crt, ter, mhpF, and fucO. In an aspect, the disclosed aerobic hydrogen bacteria comprise hbd, crt, ter, mhpF, fucO, and yqeF. In an aspect, the disclosed aerobic hydrogen bacteria comprise atoB, hbd, crt, ter, and Ma2507. In an aspect, the disclosed aerobic hydrogen bacteria comprise atoB, crt, ter, adheE2, and fadB.

In an aspect, a culture comprising a plurality of the aerobic hydrogen bacteria produces and secretes n-butanol. In an aspect, the aerobic hydrogen bacteria disclosed herein produces n-butanol when cultured in the presence of oxygen, hydrogen, and carbon dioxide and in the dark. In an aspect, the aerobic hydrogen bacteria are isolated.

In an aspect, the aerobic hydrogen bacteria of the disclosed method further comprise one or more endogenous genes that is silenced or knocked out. In an aspect, the one or more silenced or knocked out genes encode a peptide capable of converting (i) acetyl-CoA to acetoacetyl-CoA, (ii) acetoacetyl-CoA to β-hydroxybutyryl-CoA, or (iii) β-hydroxybutyryl-CoA to polyhydroxyalkanoate. In an aspect, the one or more endogenous gene that is knocked out or silenced is selected from the group consisting of phaA, phaB 1, phaC1, or phaC2. In an aspect, the construct for the phaC1 knockout comprises SEQ ID NO: 37. In an aspect, the construct for the phaC1/phaA/phaB1 knockout comprises SEQ ID NO: 38.

In an aspect, the aerobic hydrogen bacteria of the disclosed method further comprise one or more endogenous genes that is silenced or knocked out. In an aspect, the one or more silenced or knocked out genes encode phosphate acetyltransferase. In an aspect, the one or more silenced or knocked out genese encode acetate kinase. In an aspect, the construct for the ptal/ackA knockout comprises SEQ ID NO: 39.

Disclosed herein is a method of producing n-butanol, comprising (a) culturing a population of aerobic hydrogen bacteria autotrophically, wherein (i) the aerobic hydrogen bacteria comprises a genetic modification, wherein the genetic modification comprises one or more mutations in a gene encoding a ribulose bisphosphate carboxylase peptide, (ii) the carbon source comprises CO₂, and (b) recovering the n-butanol from the medium.

In an aspect, the aerobic hydrogen bacteria or the disclosed methods are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the mutated ribulose bisphosphate carboxylase peptide increases the efficiency of the protein to fix CO₂ In an aspect, the mutated ribulose bisphosphate carboxylase peptide decreases the sensitivity of the protein to O₂. In an aspect, the ribulose bisphosphate carboxylase peptide both increases the efficiency of the protein to fix CO₂ and decreases the

In an aspect, the mutated ribulose bisphosphate carboxylase peptide of the aerobic hydrogen bacteria is mutated. In an aspect, the mutated ribulose bisphosphate carboxylase peptide of the aerobic hydrogen bacteria is mutated in such a way that it results in a codon change in the wild-type sequence. For example, disclosed herein are aerobic hydrogen bacteria comprising a codon change in SEQ ID NO: 24. In an aspect, the codon change is from GGC to GGT at position 264. In an aspect, the codon change is from TCG to ACC at position 265. In an aspect, the change is S265T (SEQ ID NO: 25). In an aspect, the codon change is from GAC to GAT at position 271. In an aspect, the codon change is from GTG to GGC at position 274. In an aspect, the change is V274G (SEQ ID NO: 26). In an aspect, the codon change is from TAC to GTC at position 347. In an aspect, the change is Y347V (SEQ ID NO: 27). In an aspect, the codon change is from GCC to GTC at position 380. In an aspect, the change is A380V (SEQ ID NO: 28). In an aspect, the mutated ribulose bisphosphate carboxylase peptide comprises a combination of codon changes selected from the following: from GGC to GGT at position 264, from TCG to ACC at position 265, from GAC to GAT at position 271, from GTG to GGC at position 274, from TAC to GTC at position 347, and from GCC to GTC at position 380.

In an aspect, a culture comprising a plurality of the aerobic hydrogen bacteria produces and secretes n-butanol. In an aspect, the aerobic hydrogen bacteria disclosed herein produces n-butanol when cultured in the presence of oxygen, hydrogen, and carbon dioxide and in the dark. In an aspect, the aerobic hydrogen bacteria are isolated.

In an aspect, the aerobic hydrogen bacteria of the disclosed method further comprise one or more endogenous genes that is silenced or knocked out. In an aspect, the one or more silenced or knocked out genes encode a peptide capable of converting (i) acetyl-CoA to acetoacetyl-CoA, (ii) acetoacetyl-CoA to β-hydroxybutyryl-CoA, or (iii) β-hydroxybutyryl-CoA to polyhydroxyalkanoate. In an aspect, the one or more endogenous gene that is knocked out or silenced is selected from the group consisting of phaA, phaB 1, phaC1, or phaC2. In an aspect, the construct for the phaC1 knockout comprises SEQ ID NO: 37. In an aspect, the construct for the phaC1/phaA/phaB1 knockout comprises SEQ ID NO: 38.

Disclosed herein is a method of producing n-butanol, comprising (a) culturing a population of aerobic hydrogen bacteria autotrophically, wherein (i) the aerobic hydrogen bacteria comprises a genetic modification, wherein the genetic modification comprises one or more mutations in a gene encoding a CbbR peptide, (ii) the carbon source comprises CO₂, and (b) recovering the n-butanol from the medium.

In an aspect, the aerobic hydrogen bacteria or the disclosed methods are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the mutated CbbR peptide is constitutively active. In an aspect, the mutated CbbR peptide is more active than a wild-type CbbR peptide or a non-mutated CbbR peptide.

In an aspect, the mutated CbbR peptide of the aerobic hydrogen bacteria is mutated. In an aspect, the mutated CbbR peptide of the aerobic hydrogen bacteria is mutated in such a way that it results in a codon change in the wild-type sequence. For example, disclosed herein are aerobic hydrogen bacteria comprising a codon change in SEQ ID NO: 1. In an aspect, the amino acid mutation is L79F. (SEQ ID NO: 2). In an aspect, the amino acid mutation is E87K. (SEQ ID NO: 3). In an aspect, the amino acid mutation is E87K/G242S. (SEQ ID NO: 4). In an aspect, the amino acid mutation is G98R. (SEQ ID NO: 5). In an aspect, the amino acid mutation is A117V. (SEQ ID NO: 6). In an aspect, the amino acid mutation is G125D. (SEQ ID NO: 7). In an aspect, the amino acid mutation is G125S/V265M. (SEQ ID NO: 8). In an aspect, the amino acid mutation is D144N. (SEQ ID NO: 9). In an aspect, the amino acid mutation is D148N. (SEQ ID NO: 10). In an aspect, the amino acid mutation is A167V. (SEQ ID NO: 11). In an aspect, the amino acid mutation is G205D. (SEQ ID NO: 12). In an aspect, the amino acid mutation is G205S. (SEQ ID NO: 23). In an aspect, the amino acid mutation is G205D/G118D. (SEQ ID NO: 13). In an aspect, the amino acid mutation is G205D/R283H. (SEQ ID NO: 14). In an aspect, the amino acid mutation is P221S. (SEQ ID NO: 15). In an aspect, the amino acid mutation is P221S/T299I. (SEQ ID NO: 16). In an aspect, the amino acid mutation is T232A. (SEQ ID NO: 17). In an aspect, the amino acid mutation is T232I. (SEQ ID NO: 18). In an aspect, the amino acid mutation is P269S. (SEQ ID NO: 19). In an aspect, the amino acid mutation is P269S/T299I. (SEQ ID NO: 20). In an aspect, the amino acid mutation is R272Q. (SEQ ID NO: 21). In an aspect, the amino acid mutation is G80D/S106N/G261E. (SEQ ID NO: 22). In an aspect, the mutated CbbR peptide comprises a combination of codon changes selected from the following: L79F, E87K, E87K/G242S, G98R, A117V, G125D, G125S/V265M, D144N, D148N, A167V, G205D, G205S, G205D/G118D, G205D/R283H, P221S, P221S/T299I, T232A, T232I, P269S, P269S/T299I, R272Q, and G80D/S106N/G261E.

In an aspect, a culture comprising a plurality of the aerobic hydrogen bacteria produces and secretes n-butanol. In an aspect, the aerobic hydrogen bacteria disclosed herein produces n-butanol when cultured in the presence of oxygen, hydrogen, and carbon dioxide and in the dark. In an aspect, the aerobic hydrogen bacteria are isolated.

In an aspect, the aerobic hydrogen bacteria of the disclosed method further comprise one or more endogenous genes that is silenced or knocked out. In an aspect, the one or more silenced or knocked out genes encode a peptide capable of converting (i) acetyl-CoA to acetoacetyl-CoA, (ii) acetoacetyl-CoA to β-hydroxybutyryl-CoA, or (iii) β-hydroxybutyryl-CoA to polyhydroxyalkanoate. In an aspect, the one or more endogenous gene that is knocked out or silenced is selected from the group consisting of phaA, phaB 1, phaC1, or phaC2. In an aspect, the construct for the phaC1 knockout comprises SEQ ID NO: 37. In an aspect, the construct for the phaC1/phaA/phaB1 knockout comprises SEQ ID NO: 38.

Disclosed herein is a method of producing n-butanol, the method comprising cultivating aerobic hydrogen bacteria in a medium, wherein the aerobic hydrogen bacteria comprise (i) one or more exogenous genes, (ii) one or more mutations in a nucleic acid sequence that encodes a ribulose bisphosphate carboxylase peptide, or (iii) one or more mutations in a nucleic acid sequence that encodes a CbbR peptide; recovering the aerobic hydrogen bacteria from the medium; and recovering the n-butanol from the medium.

In an aspect, the one or more exogenous nucleic acid molecules encoding a naturally occurring polypeptide comprise ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof.

In an aspect, the aerobic hydrogen bacteria of the disclosed method are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the mutated ribulose bisphosphate carboxylase peptide of the aerobic hydrogen bacteria is mutated. In an aspect, the mutated ribulose bisphosphate carboxylase peptide of the aerobic hydrogen bacteria is mutated in such a way that it results in a codon change in the wild-type sequence. For example, disclosed herein are aerobic hydrogen bacteria comprising a codon change in SEQ ID NO: 24. In an aspect, the codon change is from GGC to GGT at position 264. In an aspect, the codon change is from TCG to ACC at position 265. In an aspect, the change is S265T (SEQ ID NO: 25). In an aspect, the codon change is from GAC to GAT at position 271. In an aspect, the codon change is from GTG to GGC at position 274. In an aspect, the change is V274G (SEQ ID NO: 26). In an aspect, the codon change is from TAC to GTC at position 347. In an aspect, the change is Y347V (SEQ ID NO: 27). In an aspect, the codon change is from GCC to GTC at position 380. In an aspect, the change is A380V (SEQ ID NO: 28). In an aspect, the mutated ribulose bisphosphate carboxylase peptide comprises a combination of codon changes selected from the following: from GGC to GGT at position 264, from TCG to ACC at position 265, from GAC to GAT at position 271, from GTG to GGC at position 274, from TAC to GTC at position 347, and from GCC to GTC at position 380.

In an aspect, the mutated CbbR peptide of the aerobic hydrogen bacteria is mutated. In an aspect, the mutated CbbR peptide of the aerobic hydrogen bacteria is mutated in such a way that it results in a codon change in the wild-type sequence. For example, disclosed herein are aerobic hydrogen bacteria comprising a codon change in SEQ ID NO:1. In an aspect, the amino acid mutation is L79F. (SEQ ID NO: 2). In an aspect, the amino acid mutation is E87K. (SEQ ID NO: 3). In an aspect, the amino acid mutation is E87K/G242S. (SEQ ID NO: 4). In an aspect, the amino acid mutation is G98R. (SEQ ID NO: 5). In an aspect, the amino acid mutation is A117V. (SEQ ID NO: 6). In an aspect, the amino acid mutation is G125D. (SEQ ID NO: 7). In an aspect, the amino acid mutation is G125S/V265M. (SEQ ID NO: 8). In an aspect, the amino acid mutation is D144N. (SEQ ID NO: 9). In an aspect, the amino acid mutation is D148N. (SEQ ID NO: 10). In an aspect, the amino acid mutation is A167V. (SEQ ID NO: 11). In an aspect, the amino acid mutation is G205D. (SEQ ID NO: 12). In an aspect, the amino acid mutation is G205S. (SEQ ID NO: 23). In an aspect, the amino acid mutation is G205D/G118D. (SEQ ID NO: 13). In an aspect, the amino acid mutation is G205D/R283H. (SEQ ID NO: 14). In an aspect, the amino acid mutation is P221S. (SEQ ID NO: 15). In an aspect, the amino acid mutation is P221S/T299I. (SEQ ID NO: 16). In an aspect, the amino acid mutation is T232A. (SEQ ID NO: 17). In an aspect, the amino acid mutation is T232I. (SEQ ID NO: 18). In an aspect, the amino acid mutation is P269S. (SEQ ID NO: 19). In an aspect, the amino acid mutation is P269S/T299I. (SEQ ID NO: 20). In an aspect, the amino acid mutation is R272Q. (SEQ ID NO: 21). In an aspect, the amino acid mutation is G80D/S106N/G261E. (SEQ ID NO: 22). In an aspect, the mutated CbbR peptide comprises a combination of codon changes selected from the following: L79F, E87K, E87K/G242S, G98R, A117V, G125D, G125S/V265M, D144N, D148N, A167V, G205D, G205S, G205D/G118D, G205D/R283H, P221S, P221S/T299I, T232A, T232I, P269S, P269S/T299I, R272Q, and G80D/S106N/G261E.

Disclosed herein is a process for preparing n-butanol, the process comprising providing a culture, the culture comprising aerobic hydrogen bacteria comprising (i) one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or a combination thereof, (ii) a genetic modification, wherein the genetic modification comprises one or more mutations in a gene encoding a ribulose bisphosphate carboxylase peptide, and (iii) a genetic modification, wherein the genetic modification comprises one or more mutations in a gene encoding a CbbR peptide; culturing the aerobic hydrogen bacteria in the dark and in the presence of oxygen, hydrogen, and carbon dioxide; and recovering the n-butanol from the culture.

In an aspect, the aerobic hydrogen bacteria of the disclosed method are the species Ralstonia eutropha, Rhodobacter capsulatus, or Rhodobacter sphaeroides. In an aspect, the aerobic hydrogen bacteria disclosed herein belong to the Pseudomonas genera. In an aspect, the disclosed aerobic hydrogen bacteria are actinobacteria. In an aspect, the aerobic hydrogen bacteria disclosed herein are carboxidobacteria. In an aspect, the disclosed aerobic hydrogen bacteria are nonsulfur purple bacteria including but not limited to the families Rhodospirillales and Rhizobiales. In an aspect, the family Rhodospirillales comprises Rhodospirillaceae (e.g., Rhodospirillum) and Acetobacteraceae (e.g., Rhodopila). In an aspect, the family Rhizobiales comprises Bradyrhizobiaceae (e.g., Rhodopseudomonas palustris), Hyphomicrobiaceae (e.g., Rhodomicrobium), and Rhodobacteraceae (e.g., Rhodobium). In an aspect, other families of nonsulfur purple bacteria comprise Rhodobacteraceae (e.g., Rhodobacter), Rhodocyclaceae (e.g., Rhodocylus), and Comamonadaceae (e.g., Rhodoferax).

In an aspect, the mutated ribulose bisphosphate carboxylase peptide of the aerobic hydrogen bacteria is mutated. In an aspect, the mutated ribulose bisphosphate carboxylase peptide of the aerobic hydrogen bacteria is mutated in such a way that it results in a codon change in the wild-type sequence. For example, disclosed herein are aerobic hydrogen bacteria comprising a codon change in SEQ ID NO: 24. In an aspect, the codon change is from GGC to GGT at position 264. In an aspect, the codon change is from TCG to ACC at position 265. In an aspect, the change is S265T (SEQ ID NO: 25). In an aspect, the codon change is from GAC to GAT at position 271. In an aspect, the codon change is from GTG to GGC at position 274. In an aspect, the change is V274G (SEQ ID NO: 26). In an aspect, the codon change is from TAC to GTC at position 347. In an aspect, the change is Y347V (SEQ ID NO: 27). In an aspect, the codon change is from GCC to GTC at position 380. In an aspect, the change is A380V (SEQ ID NO: 28). In an aspect, the mutated ribulose bisphosphate carboxylase peptide comprises a combination of codon changes selected from the following: from GGC to GGT at position 264, from TCG to ACC at position 265, from GAC to GAT at position 271, from GTG to GGC at position 274, from TAC to GTC at position 347, and from GCC to GTC at position 380.

In an aspect, the mutated CbbR peptide of the aerobic hydrogen bacteria is mutated. In an aspect, the mutated CbbR peptide of the aerobic hydrogen bacteria is mutated in such a way that it results in a codon change in the wild-type sequence. For example, disclosed herein are aerobic hydrogen bacteria comprising a codon change in SEQ ID NO: 1. In an aspect, the amino acid mutation is L79F. (SEQ ID NO: 2). In an aspect, the amino acid mutation is E87K. (SEQ ID NO: 3). In an aspect, the amino acid mutation is E87K/G242S. (SEQ ID NO: 4). In an aspect, the amino acid mutation is G98R. (SEQ ID NO: 5). In an aspect, the amino acid mutation is A117V. (SEQ ID NO: 6). In an aspect, the amino acid mutation is G125D. (SEQ ID NO: 7). In an aspect, the amino acid mutation is G125S/V265M. (SEQ ID NO: 8). In an aspect, the amino acid mutation is D144N. (SEQ ID NO: 9). In an aspect, the amino acid mutation is D148N. (SEQ ID NO: 10). In an aspect, the amino acid mutation is A167V. (SEQ ID NO: 11). In an aspect, the amino acid mutation is G205D. (SEQ ID NO: 12). In an aspect, the amino acid mutation is G205S. (SEQ ID NO: 23). In an aspect, the amino acid mutation is G205D/G118D. (SEQ ID NO: 13). In an aspect, the amino acid mutation is G205D/R283H. (SEQ ID NO: 14). In an aspect, the amino acid mutation is P221S. (SEQ ID NO: 15). In an aspect, the amino acid mutation is P221S/T299I. (SEQ ID NO: 16). In an aspect, the amino acid mutation is T232A. (SEQ ID NO: 17). In an aspect, the amino acid mutation is T232I. (SEQ ID NO: 18). In an aspect, the amino acid mutation is P269S. (SEQ ID NO: 19). In an aspect, the amino acid mutation is P269S/T299I. (SEQ ID NO: 20). In an aspect, the amino acid mutation is R272Q. (SEQ ID NO: 21). In an aspect, the amino acid mutation is G80D/S106N/G261E. (SEQ ID NO: 22). In an aspect, the mutated CbbR peptide comprises a combination of codon changes selected from the following: L79F, E87K, E87K/G242S, G98R, A117V, G125D, G125S/V265M, D144N, D148N, A167V, G205D, G205S, G205D/G118D, G205D/R283H, P221S, P221S/T299I, T232A, T232I, P269S, P269S/T299I, R272Q, and G80D/S106N/G261E.

### D. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### i) EXAMPLE 1

a. Engineering Metabolic Pathways of Hydrogen Bacteria for the Production of Butanol.

To maximize butanol production, the general toxicity of butanol to various cultures of hydrogen bacteria was assessed. It was found that both Ralstonia eutropha and Rhodobacter capsulatus tolerate up to about 0.8% butanol before growth was affected. It was also found that this toxicity was a reversible process, so that once butanol is removed from cultures, the organisms recovered, retained viability, and continued to grow as before. This reversibility of the potential toxic effects of accumulated butanol is a consideration for large scale bioreactors and maximizes the recovery of butanol from fermentation broths. Mutant strains that are more resistant to butanol were also developed.

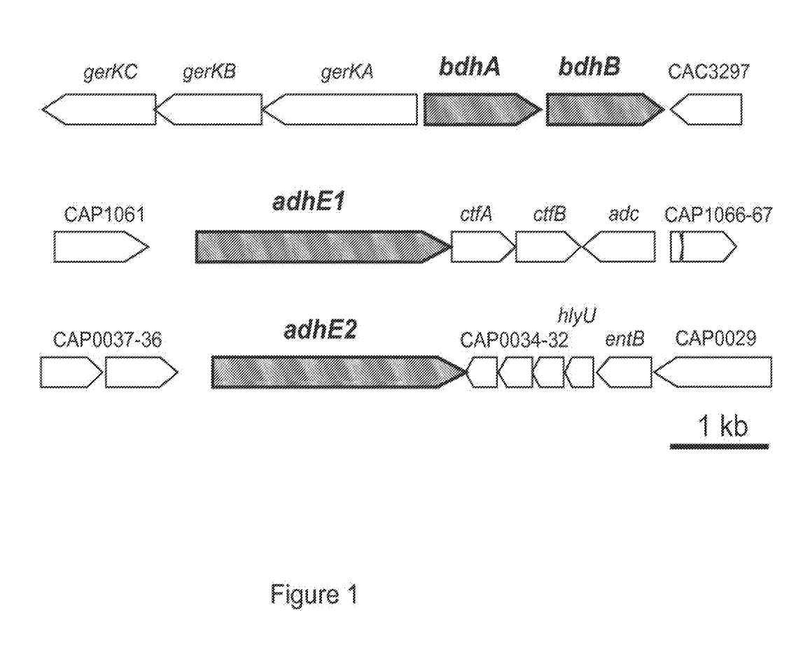

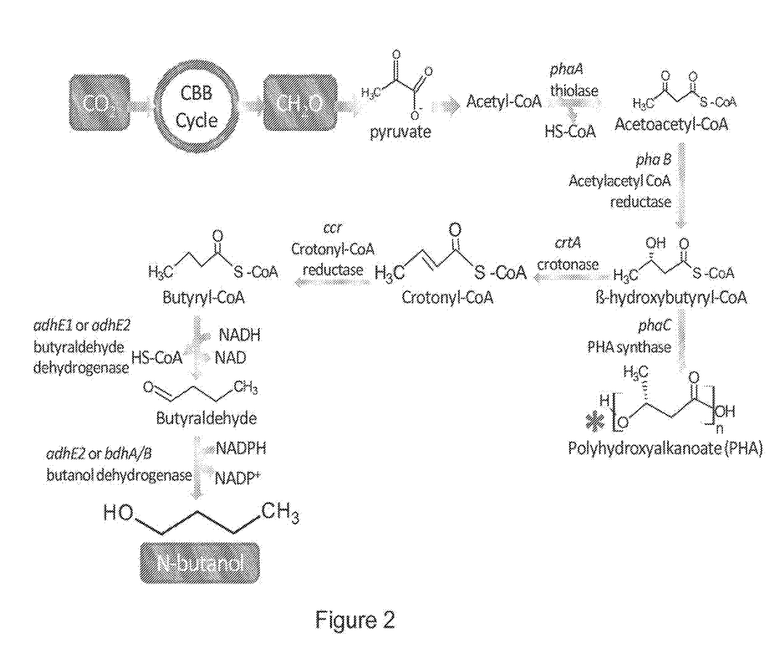

Using novel vectors, several different butanol genes from Clostridium acetobutylicum were introduced into both Rhodobacter capsulatus and Ralstonia eutropha. The genes include the bdhA/bdhB, adhE1, and adhE2 genes as indicated in FIG. 1. The adhE2 gene was expressed by over 10-fold over controls, as shown by the transfer of the plasmid containing this gene into one of the target hydrogen bacteria.

b. Engineering the Metabolic Regulation of the Calvin Cycle for Constitutive Carbon Fixation Under All Growth Conditions.

Biochemical and molecular approaches were utilized to analyze the in vitro CbbR function of R. eutropha. These studies aimed to make CbbR constitutively active so that under any growth condition CbbR could activate cbb gene expression. This, in turn, would keep the CO₂ fixation genes in an up-regulated mode. Unless there are extra reducing equivalents available, the reducing power for maximum butanol production may become limiting with synthetic organisms. An effective way to provide extra reducing equivalents is to add organic carbon, which typically results in repression of the cbb genes. However, a constitutively active CbbR molecule obviates organic-carbon mediated repression, thereby ensuring that the CO₂ fixation (cbb) genes are always highly expressed regardless of the provision of carbon.

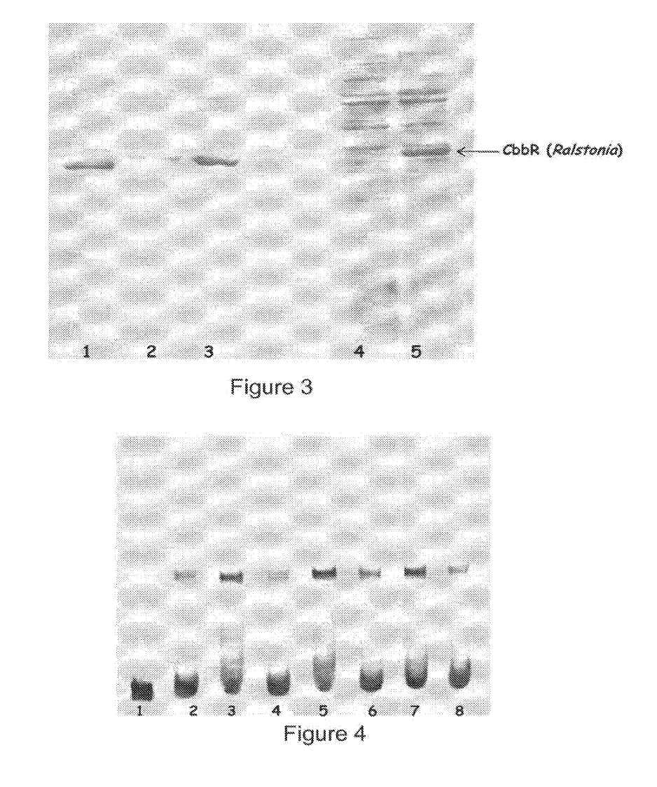

Properly folded and active CbbR was isolated for in vitro experiments. Actual achieved levels of active CbbR represented over 20% of the total soluble protein. These results are shown in FIG. 3. The purified recombinant CbbR preparations were tested for activity in binding to specific promoter sequences from R. eutropha. As shown by gel mobility shift assays, the purified recombinant CbbR was active. Specific promoter DNA sequence was labeled with [³²P] were shown to bind to the recombinant CbbR protein, which was illustrated by its ability to bind to the labeled probe and cause a shift in mobility in a native polyacrylamide gel (FIG. 4).

The results of these experiments indicated that various effectors, namely RuBP, PEP, and ATP, enhanced CbbR binding to the probe (FIG. 4). Thus, the constitutively active R. eutropha CbbR could be isolated via a similar mutagenesis approach (i.e., to identify CbbR proteins that are indifferent to the presence of positive or negative effectors). Such proteins, when incorporated into R. eutropha, would allow high level cbb transcription under all conditions of growth, thereby facilitating efforts to achieve maximum production of n-butanol.

### ii) EXAMPLE 2

a. Engineering Metabolic Pathways of Hydrogen Bacteria for the Production of Butanol.

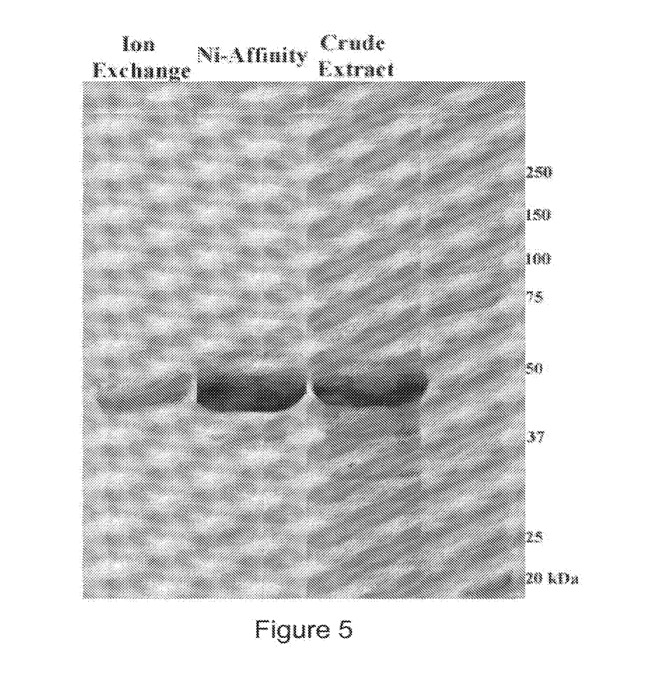

Highly purified recombinant RubisCO was prepared from Ralstonia eutropha. Recombinant RubisCO allowed for the enzyme to be more productive in CO₂ fixation, which resulted in a greater production of n-butanol from CO₂. The recombinant RubisCO was ≧95 percent pure (FIG. 5).

In terms of potentially enhancing CO₂ fixation in R. eutropha, kinetic analyses indicated that the recombinant RubisCO enzyme was especially adapted for aerobic CO₂ fixation. Here, the ratio of its affinities for O₂ and CO₂ (Kₒ/K_{c}) was very high in comparison to both the wild-type and the mutant (A375V) cyanobacterial RubisCO. The specificity factor (a measure of the efficiency for CO₂ fixation) was also considerably higher for the R. eutropha enzyme (Table 1).

Table 1 shows the kinetic properties of R. eutropha RubisCO as compared to the wild-type cyanobacterial enzyme and a mutant form of cyanobacterial RubisCO (A375V). The mutant form of RubisCO (A375V) was better able to support aerobic CO₂ fixation than the wild type cyanobacterial RubisCO enzyme.

TABLE 1

| | K_{cat} | K_{C} | K_{O} | | Specificity |
|---|---|---|---|---|---|
| Enzyme | (s−1) | (μM CO₂) | (μM O₂) | K_{O}/K_{C} | Factor |
| Wild Type | 7.1 | 234 | 978 | 4.2 | 43 |
| A375V | 0.8 | 171 | 1294 | 7.6 | — |
| Ralstonia RubisCO | 3.4 | 50 | 1293 | 25.9 | 83 |

Several different genes that encode butanol dehydrogenase activity from Clostridium acetobutylicum were inserted into Rhodobacter capsulatus or Rb. sphaeroides and R. eutropha and subsequently analyzed. The ability of various promoter/vector constructs to maximize expression of the genes of interest (e.g., butanol dehydrogenase, including the bdhA/B and adhE1/adhE2 genes from C. acetobutylicum) were also analyzed. The first promoter/vector construct to be examined were highly regulated and very active when CO₂ was used as the carbon source in Rhodobacter for expressing exogenous genes, including genes for ethanol production.

Table 2 shows the results of those experiments in which the adhE2 gene was expressed in R. eutropha under both aerobic chemoheterotrophic and aerobic chemoautotrophic growth conditions (i.e., using CO₂ as sole carbon source). Similar results were obtained using this promoter/vector construct and the bdhA/B genes in R. eutropha. Table 2 also shows the RT-PCR analysis of the amount of DNA synthesized from adhE2 transcripts in wild type R. eutropha grown chemoheterotrophically (CH) and chemoautotrophically (CA). To determine the presence of contaminating DNA, controls were performed without reverse transcriptase. The amount of DNA synthesized was measured of the level of gene transcription (amount of transcript produced) under the two growth conditions.

TABLE 2

| | ng DNA/ |
|---|---|
| Sample | ng total RNA |
| CH cells, no plasmid | 0 |
| CA cells, no plasmid | 0 |
| CH cells plus adhE2 containing plasmid | 775 |
| CH cells plus adhE2 containing plasmid | 0 |
| minus reverse transcriptase | |
| CA cells plus adhE2 containing plasmid | 680 |
| CA cells plus adhE2 containing plasmid | 0 |
| minus reverse transcriptase | |

b. Engineering the Metabolic Regulation of the Calvin Cycle for Constitutive Carbon Fixation Under All Growth Conditions.

Large amounts of properly folded and active recombinant CbbR were isolated for in vitro experiments. As shown by gel mobility shift assays using [³²P]-labeled promoter DNA, these CbbR preparations were active in binding specific DNA promoter sequences. It was also found that various potential positive and negative effectors influenced CbbR binding. The presence of organic carbon typically leads to repression of CO₂ fixation gene expression. Therefore, the effect of various positive and negative effectors is a consideration in preparing constitutively active CbbR proteins that are indifferent to the presence of effectors. It is desirable that the CO₂ fixation genes remain up-regulated, thereby allowing n-butanol synthesis from CO₂ in the presence of organic compounds that can supply necessary reductant to the cells.

Positive and negative effectors that influence CbbR binding and activity in vitro were studied. Such effectors, which are generated as a result of cell metabolism, can influence CbbR function in vivo as well as the subsequent expression of CO₂ fixation genes. Various mutations in CbbR function have been isolated and these mutations abrogate the ability of effectors to influence CbbR function both in vitro and in vivo. The net effect was to allow CO₂ fixation gene expression to be up-regulated under various types of growth conditions.

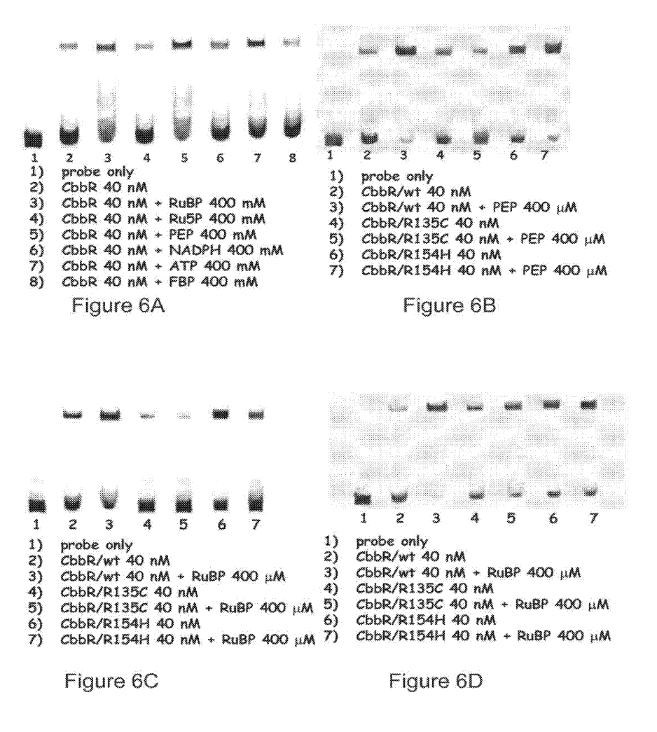

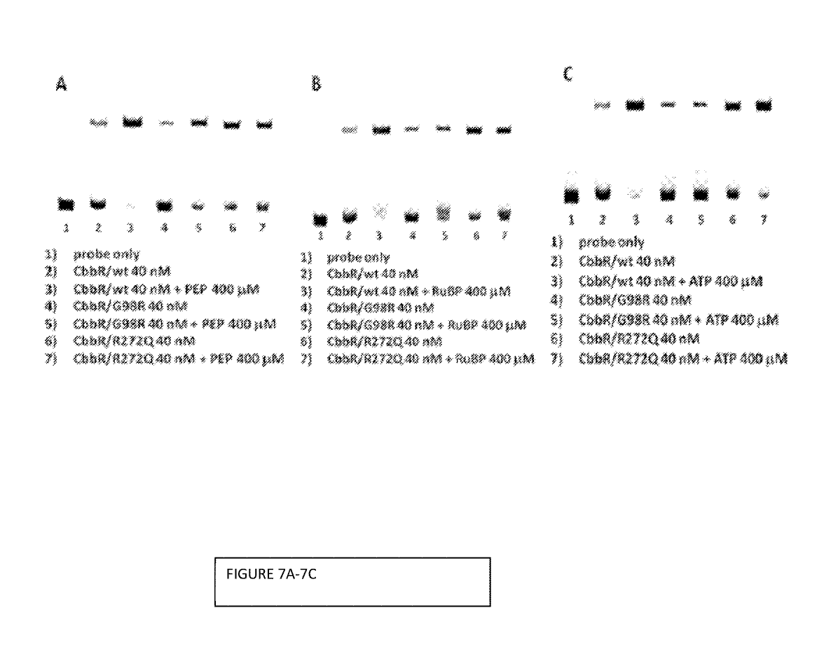

FIG. 6 and FIG. 7 show the data generated by electrophoretic gel mobility shift assays. Here, the assays were used with purified R. eutropha CbbR to determine whether effectors such as RuBP, PEP, and ATP influenced CbbR binding to a specific cbb promoter sequence. The effect of various mutations on CbbR binding was also characterized. The results indicated that R. eutropha CbbR was subject to effector-mediated enhancement binding to its specific promoter sequence and that various site-directed mutations influenced this binding. The results are summarized in Table 3, which shows the fold changes in CbbR binding affinity for the cbb promoter in the presence of the metabolite (400 μM) relative to CbbR binding affinity in the absence of the metabolite.

TABLE 3

| CbbR mutant | PEP | RuBP | ATP | NADPH | RU5P | FBP |
|---|---|---|---|---|---|---|
| Wt | 3.8 | 2.3 | 3.2 | 1.5 | 0.91 | 0.96 |
| G98R | 2.7 | 1.2 | 0.99 | | | |
| R135C | 0.97 | 0.59 | 1.3 | | | |
| R154H | 1.3 | 0.68 | 1.2 | | | |
| R272O | 0.85 | 0.76 | 1.4 | | | |

### iii) EXAMPLE 3

a. Engineering Metabolic Pathways of Hydrogen Bacteria for the Production of Butanol.

When the Clostridium acetobutylicum adhE2 gene was successfully expressed in R. eutropha, R. eutropha synthesized butanol. The addition of the adhE2 gene provided R. eutropha with a complete pathway for butanol production. Thus, systematic efforts to optimize and improve butanol production by aerobic hydrogen bacteria, such as R. eutropha, were undertaken. The strategy included (1) the optimization of gene expression and protein synthesis, (2) the introduction of a synthetic butanol pathway to supplement the native catalysts that lead to the starting material for butanol synthesis, and (3) the removal of one or more potentially competing pathways.

To increase butanol production, several promoters (e.g., lac, tac, cbbM, cbbL, and pha) were examined to identify the promoter that produced the best overall expression of the butanol production genes. The lac and tac promoters are E. coli promoters, but have been used to drive gene expression of other genes in R. eutropha. The pha promoter is a native R. eutropha promoter and drives expression of genes involved in polyhydroxybutyrate (PHB) production. The relative strength of these promoters in R. eutropha was determined. The pha promoter was 1.2 times stronger than the lac promoter and that the tac promoter was 2.1 times stronger than the lac promoter (1). The cbbM and cbbL promoters were also examined. The cbbM and cbbL promoters are strong promoters which drive expression of the genes that encode for RubisCO in Rhodosporilium rubrum/Rhodobacter sphaeroides/Rhodobacter capsulatus and R. eutropha, respectively. To further increase protein synthesis, a R. eutropha optimized ribosome binding site (RBS) was included immediately upstream of each butanol production gene. Each promoter was placed in the vector pBBR1MCS3, and the ability of these gene expression vectors was assessed (Table 4). The pBBR1 vector has Accession No. U02374 (4707 bp). The pBBR1MCS-3 vector has Accession No. U25059 (5228 bp). Plasmid pRPS-MCS3 (SEQ ID NO: 36) (see Journal of Molecular Biology, 331(3): 557-569 (2003)) derives from plasmid pBBR1-MCS3.

TABLE 4

| Promoter | Source |
|---|---|
| cbbM | Rhodosporiium rubrum |
| lac | Escherichia coli |
| tac | synthetic |
| cbbL | Ralstonia eutropha |
| pha | Ralstonia eutropha |

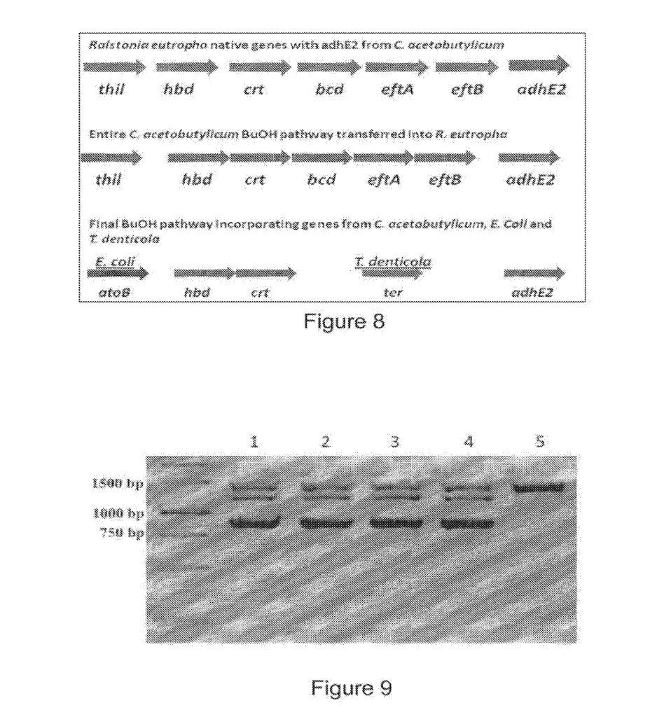

Previously, the production of butanol in R. eutropha was reliant on native gene products that were able to convert two acetyl-CoA molecules to butyryl-CoA. This conversion was followed by the conversion of butyryl-CoA to butanol by the protein encoded by the exogenous C. acetobutylicum adhE2 gene. However, to improve butanol production, a set of C. acetobutylicum genes (e.g., thil, hbd, crt, bcd, etfA, etfB and adhE2) were cloned into R. eutropha. The effect of different promoters on the expression of this pathway was examined (Table 5). Furthermore, in addition to cloning genes from C. acetobutylicum into R. eutropha, the genes from two other organisms were examined. The first gene was the atoB gene from E. coli. The atoB enzyme demonstrated five times higher catalytic activity than the C. acetobutylicum thil enzyme (Shen et al., 2011). atoB was substituted for thil in the synthetic butanol pathway (FIG. 8). This increased the rate of the first reaction in the butanol pathway. The second gene was the ter gene from Treponema denticola. The ter gene replaced the bcd, etfA and etfB genes from C. acetobutylicum. The ter gene product had two distinct advantages. First, it was not oxygen sensitive (which differed from that of the bcd-eftAB gene product complex). Second, the ter gene product catalyzed the conversion of crotonyl-CoA to butyryl-CoA in a non-reversible manner (which differed from that of the bcd-eftAB complex). The use of the ter gene product drove the flux in the direction of butanol production and prevented the pathway from going in the opposite direction. Table 5 shows a summary of the cloning butanol production genes in R. eutropha. In addition to these constructs, the entire native C. acetobutylicum suite of genes was cloned into R. eutropha and was compared to results obtained with the mixture of genes from the three organisms.

TABLE 5

| Promoter | Genes |
|---|---|
| lac | adhE2 hbd crt, ter, adhE2, atoB |
| tac | adhE2 hbd crt, ter, adhE2, ato B |
| cbbM | adhE hbd crt, ter, adhE2, atoB |
| cbbL | adhE2 hbd, crt, ter, adhE2, atoB |
| pha | adhE2 hbd, crt, ter, adhE2, atoB |

Another method for increasing butanol production was to increase metabolic flux in the direction of the butanol pathway in R. eutropha. This was accomplished by removing the competing PHB pathway. The butanol and PHB pathways both share the same starting substrate, acetoacetyl-CoA. In R. eutropha, the PHB pathway is encoded by the phaCAB operon. In order to inactivate the PHB production pathway, a gene knockout vector was created that targets the phaC gene. This vector was introduced into R. eutropha, and a partial R. eutropha phaC deletion strain was created (FIG. 9).

b. Engineering the Metabolic Regulation of the Calvin Cycle for Constitutive Carbon Fixation Under All Growth Conditions.

The enzymes and molecular regulator proteins of the Calvin-Benson-Bassham (CBB) CO₂ fixation pathway are considerations in any effort to maximize the bioconversion of CO₂ to desired products, such as butanol, via the synthetic pathway described above. The key transcriptional regulator that controls the expression of genes (cbb) required for CO₂ assimilation is CbbR, encoded by a gene (cbbR) that is divergently transcribed from the cbb operon. Prior studies with other hydrogen bacteria have shown that mutant CbbR proteins can be used to enhance cbb gene expression, as well as allow for cbb gene expression under cellular growth conditions when CbbR is normally ineffective in up-regulating gene expression. CbbR is a transcription factor that is required for expression of genes involved in CO₂ fixation. Recombinant CbbR proteins have been isolated for in vitro studies. The ability of various cellular metabolites (effectors) to influence CbbR binding to its specific target (promoter) DNA has also been characterized. CbbR has been expressed in R. eutropha under the control of various different promoter/vector constructs. RubisCO, the key and rate limiting CBB pathway enzyme, has also been improved so that it is a more effective catalyst for driving CO₂ conversion to product.

To identify constitutive mutations in the CbbR protein, the deletion of the native wild-type cbbR gene from R. eutropha was first undertaken. A cbbR knock-out strain of Ralstonia eutropha was the first step in generating a reporter strain for the identification of CbbR constitutive mutants. Once cbbR was nonfunctional, a reporter plasmid containing the lacZ gene driven by the cbb promoter was integrated into the Ralstonia genome at the cbbR gene deletion locus. This reporter strain was then used to identify mutants of CbbR that constitutively activate the cbb operon under chemoheterotrophic conditions and also increased expression of the cbb operon under chemoautotrophic conditions.

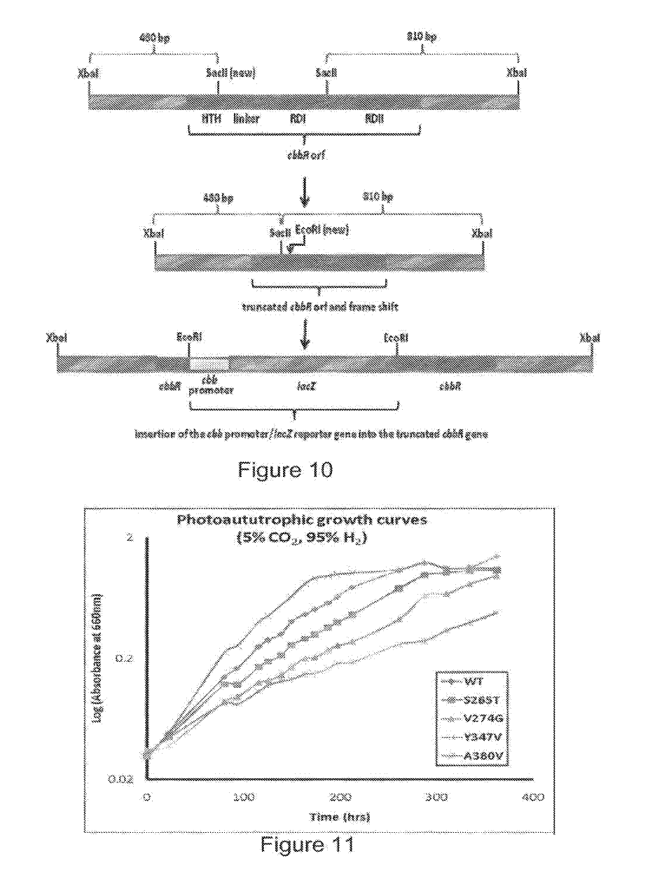

The strategy for creating a cbbR knock-out in R. eutropha was to delete 380 bp of the cbbR gene, which generated a frame-shift downstream of the deletion (FIG. 10). This kept the cbb promoter intact while creating a nonfunctional CbbR. A SacII site was created at the 5′ end of the cbbR orf. A second SacII site already existed 528 bp into the orf of cbbR. DNA between the two SacII sites was deleted and this construct was placed into a suicide vector (pJQ/RKO) and mated into strain H16 (R. eutropha). Double recombinants that had the deletion plus frame-shifted cbbR gene in place of the wild-type gene on the chromosome were selected (by PCR and sequencing). Thus, a cbbR knock-out strain for R. eutropha was successfully isolated. The final step in generating a reporter strain was to insert a cbb promoter/lacZ reporter gene into the Ralstonia genome using the suicide vector, pJQ, which contained the cbb/lacZ gene inserted into the truncated cbbR gene at a newly created EcoRI site (FIG. 10). This construct integrated into the Ralstonia genome at the deleted cbbR locus and provided a means for identification of CbbR mutants that activated the cbb operon under chemoheterotrophic growth conditions. Accordingly, a R. eutropha reporter strain that turns cells (colonies) blue on X-gal indicator plates when the cbb promoter is activated was created. This reported strain allowed previously defined mutant CbbR proteins to/be expressed in the R. eutropha host organism.

The rbcLS gene cluster from Ralstonia eutropha megaplasmid pMG1 was cloned, expressed in E. coli, and then purified to homogeneity. Baseline kinetic properties were determined from the recombinant R. eutropha RubisCO. Functional competency was demonstrated in vivo by transferring these genes into a RubisCO-deletion strain of Rhodobacter capsulatus (strain SB I/II-). For a discussion of SB I/II-, see Journal of Bacteriology, 180(16): 4258-4269 (1998). Aiming to increase the enzyme's net CO₂-fixation ability for channeling more carbon into the biosynthetic pathway for butanol production, substitutions in the Ralstonia enzyme that would confer less sensitivity to O₂ were identified and engineered. Four “positive” mutant-substitutions were identified using the Synechococcus RubisCO-based bioselection system. These mutations were replicated in the Ralstonia enzyme. Whereas the Synechococcus wild-type RubisCO was unable to support oxygenic chemoautotrophic growth of R. capsulatus SBI/II-, these “positive” mutants were able to complement under these conditions. Specifically, these changes corresponded to the M259T, A269G, F342V, and A375V substitutions in the Synechococcus enzyme. The equivalent changes were S265T, V274G, Y347V, and A380V in the Ralstonia enzyme, respectively (Table 6).

TABLE 6

| RubisCO Enzymes | AA 259 | AA 269 | AA 342 | AA 375 |
|---|---|---|---|---|
| Synechococcus PCC6301 | M | A | F | A |
| Spinacea oleracea (Spinach) | V | G | F | A |
| Nicotiana tabacum | V | G | F | A |
| (Tobacco) | | | | |
| Chlamydomonas reinhardtii | V | G | F | A |
| Galdieria partita | S | I | Y | A |
| Ralstonia eutropha | S | V | Y | A |
| AA = Amino Acid | AA 265 | AA 274 | AA 347 | AA 380 |

The Y347V mutant conferred a slight growth advantage over all other RubisCOs (including the wild type). For those mutants that were able to confer growth advantage relative to the wild type, a quantitative measure of the CO₂-fixation abilities were measured directly from the growth cultures of Ralstonia. The mutants were also introduced into strain H16 (wild type), which has functional copies of both the genomic and megaplasmid RubisCOs. See Nature Biotechnology, 24(10): 1257-1262 (2006) for a discussion of the R. eutropha H16 wild-type strain. Based on growth on solid media, the mutants appeared to grow just as well as the wild-type strain.

The mutant enzymes have been expressed as recombinant enzymes in E. coli and purified using the identical procedure employed for the wild-type enzyme. Catalytic properties were determined from these enzymes using radiometric assays that measure incorporation of ¹⁴C-labeled CO₂ in the form of NaHCO₃ (Table 7). The A380V mutant enzyme showed decreased oxygen sensitivity, as seen from the initial velocity vs. CO₂ concentration plots prepared from assays carried out in the presence (100%) or absence of O₂ in the reaction vials. The oxygen insensitivity was manifested in the form of a higher Kₒ value. There was also a decrease in the enzyme's k_{cat} (Table 7).

TABLE 7

| | K_{cat} | K_{m (CO2)} | K_{m (O2)} | |
|---|---|---|---|---|
| Enzyme | (s⁻¹) | (μM) | (μM) | K_{O}/K_{C} |
| Wild Type | 3.84 ± 0.54 | 47 ± 4 | 1149 ± 56 | 24.4 |
| S265T | 3.80 ± 0.04 | 36 ± 3 | 971 ± 30 | 27.0 |
| V274C | 1.32 ± 0.16 | 36 ± 2 | 726 ± 29 | 20.2 |
| Y347V | 4.14 ± 0.66 | 45 ± 1 | 1139 ± 93 | 25.3 |
| A380V | 0.25 ± 0.04 | 34 ± 2 | 1435 ± 109 | 42.2 |

Unlike other hydrogen (photosynthetic) bacteria, Ralstonia is capable of growing rapidly in the presence of oxygen and this is indicative of RubisCO's ability to function in the presence of those oxygen levels. Ralstonia can be challenged with higher levels of oxygen and select for mutations in RubisCO genes that allow for unrestricted growth. This allows for a robust selection for RubisCO enzymes with an overall enhancement in the ability to fix carbon undeterred by the presence of O₂. Towards this end, a strain of Ralstonia was generated in which both the genomic and megaplasmid copies of the RubisCO genes were knocked out with both the 5′ and 3′ regions intact. Such an altered RubisCO can facilitate the production of desired products from CO₂ under vigorous aerobic growth conditions.

Regarding the development of solvent tolerance within the organisms to be used for butanol production, several adaptive mutants were isolated. These mutants were identified using a combination of approaches, including but not limited to EMS mutagenesis, selective pressure through exposure to increasing gas phase butanol concentrations, and adaptive evolution with an in-house developed chemostat test system designed to retain butanol. The adaptive mutants of R. eutropha H16 grew on complex solid media containing 1.2% butanol in the sealed gas mix systems, which indicated that these mutants could be transitioned away from the complex solid media to more industrially relevant media and conditions. The use of complex media allowed for the quick selection of mutants due to the increased growth rates in these situations. Now that the isolation of relevant mutants from the systems using the complex media has been accomplished, the selection of mutants for tolerance can alos occur via the use of minimal media within liquid systems. Using the chemostat test system containing minimal salts media, adaptive mutants were capable of growth at 0.7% butanol (v/v) and continued to respire up to 0.75%. Wild type R. eutropha H16 ceased growth and respiration between 0.2 and 0.3% butanol (v/v).

### iv) EXAMPLE 4

a. Engineering Metabolic Pathways of Hydrogen Bacteria for the Production of Butanol.

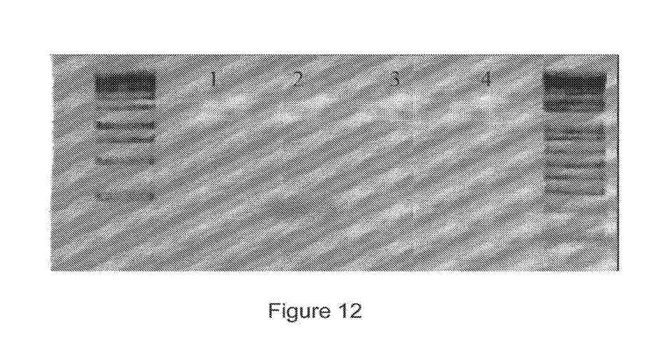

The synthesis of polyhydroxyalkonoates, such as polyhydroxyalkonoanates, such as poly-β-hydroxybutyrate (PHB), represents a major commitment of the organism to funnel carbon and reducing equivalents to storage compounds, even under conditions where CO₂ is the carbon source. Under some growth conditions, PHB synthesis can be blocked without undue hardship to the organism. Therefore, whether strains lacking the ability to synthesize PHB were more apt to funnel carbon and reducing power to desired products, such as n-butanol, was examined. The phaC1 gene is required for PHB synthesis. A gene knockout vector that targets the phaC1 gene was constructed. Such a vector allowed for the selection for a partial R. eutropha phaC1 deletion strain. The phaC1 gene was deleted and a phaC1 knockout strain was generated. This was confirmed by genomic PCR and sequencing. Based on the RT-PCR analysis, the expression of the phaC1 gene did not occur in the mutant strain (FIG. 12). This mutant strain was used to determine enhancement of the production of desired products such as n-butanol.

Promoters that drive the expression of butanol related genes for increased n-butanol production in R. eutropha were isolated. For example, the adhE2 gene driven by the cbbM promoter resulted in modest n-butanol production. Two additional promoters were examined, the lac and tac promoters. When these two promoters were used to drive adhE2 gene expression in R. eutropha, no detectable butanol was produced. Additional constructs were constructed, including a construct that utilized (1) the native cbbL, (2) the constitutive cbbL promoters, and (3) the arabinose inducible promoter (pBAD). The cbbL promoters are native to R. eutrpha. As the induction of the pBAD promoter in R. eutropha could also optimized, the pBAD promoter allowed for the regulation of gene expression of butanol production genes.

The endogenous enzymes in R. eutropha did not appear to provide enough precursor compounds to generate sufficient substrate for the recombinant butanol pathway enzymes encoded by Clostridium acetobutylicum adhE2. Thus, totally synthetic pathways in R. eutropha were produced. These pathways start from acetoacetyl-CoA (Table 8). The various synthetic pathways included genes from other organisms, which genes were previously effectively used for butanol production in non CO₂ fixing organisms. A first synthetic butanol pathway utilized (i) atoB from E. coli, (ii) hbd, crt, and adhE2 from C. acetobutylicum, and (iii) ter from T. denticola. Furthermore, each gene in this operon contained a R. eutropha optimized ribosome binding site immediately upstream of the translation start site. Results using the tac promoter to drive expression of this pathway did not provide any improvement in butanol production. RT-PCR analysis was done to verify expression of each gene in the pathway. A second synthetic pathway utilized utilized (i) atoB from E. coli, (ii) hbd and crt from C. acetobutylicum, (iii) ter from T. denticola, and (iv) mhpF and fucO from E. coli.

Historically, in biofuel studies with non CO₂ fixing organisms, the bi-functional AdhE2 enzyme was used to catalyze the in vivo conversion of butyryl-CoA to butanol with the concurrent conversion of acetyl-CoA to ethanol. The production of ethanol was greater than butanol. Recently, the use of the mhpF (aldehyde dehydrogenase) and fucO (alcohol dehydrogenase) enzymes from E. coli were used for the production of butanol (Dellomonaco et al., 2011). The production of butanol exceeded ethanol. The use of two separate enzymes (mhpF and fucO) as opposed to one (adhE2) may be responsible for the greater butanol to ethanol production ratio. These genes were cloned with the disclosed promoters to evaluate the specificity toward butanol production over ethanol production. In addition these genes were inserted in place of the adhE2 gene in the synthetic pathway, thus providing a second synthetic butanol pathway. The entire butanol synthetic pathway from C. acetobutylicum was cloned into several of the promoter/vector constructs. As the cbbM promoter is highly effective for expressing exogenous genes under CO₂ fixing growth conditions in strains of this organism, these synthetic pathways were evaluated in Rhodobacter. Table 8 shows a summary of gene, promoter, and synthetic butanol pathway constructs.

TABLE 8

| | Aldehyde/ | Aldehyde/ | | |
|---|---|---|---|---|
| | Alcohol | Alcohol | 1^{st} Synthetic | 2^{nd} Synthetic |
| Promoter | Dehydrogenases | Dehydrogenases | BuOH Pathway | BuOH Pathway |
| Tac | adhE2 | (mhpF) + (fucO) | atoB + hbd + crt + | atoB + hbd + crt + |
| | | | ter + adhE2 | ter + mhpF + fucO |
| cbbM | adhE2 | (mhpF) + (fucO) | atoB + hbd + crt + | atoB + hbd + crt + |
| | | | ter + adhE2 | ter + mhpF + fucO |
| pBAD | adhE2 | (mhpF) + (fucO) | atoB + hbd + crt + | atoB + hbd + crt + |
| | | | ter + adhE2 | ter + mhpF + fucO |

b. Engineering the Metabolic Regulation of the Calvin Cycle for Constitutive Carbon Fixation Under All Growth Conditions.

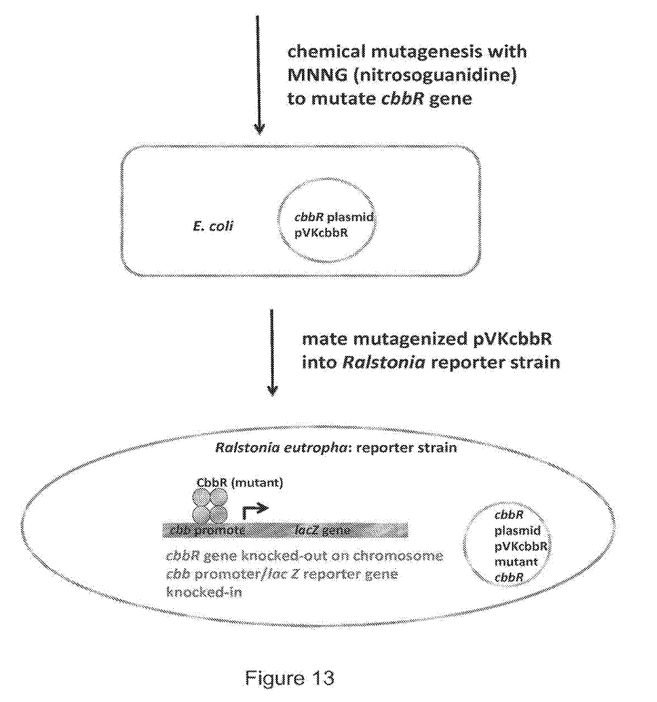

CbbR is a transcriptional regulator protein that is required for the expression of cbb genes involved in CO₂ fixation. Section for mutant CbbR proteins has occurred, which mutant proteins allow for higher expression of cbb genes (i) under growth conditions where CO₂ is the carbon source or (ii) under heterotrophic conditions where organic carbon is utilized (and normally results in repressed gene expression). Randomly mutagenesisis of cbbR DNA resulted in cbbR DNA that was cloned into an R. eutropha reporter strain constructed. The cbb promoter was linked to a lacZ gene. Thus, the appearance of blue colonies on X-gal plates was monitored when the organism was grown under normally repressive (chemoheterotrophic) growth conditions with certain sources of organic carbon (FIG. 13). Blue colonies represented mutant CbbR proteins that were constitutively active under conditions in which the wild-type CbbR protein was not active in turning on the cbb promoter (i.e.g, colonies were white on X-gal plates).

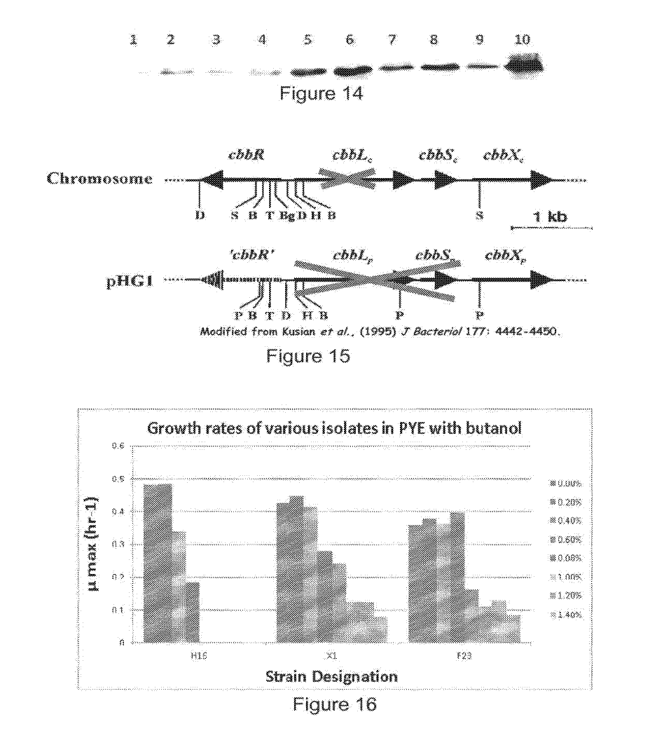

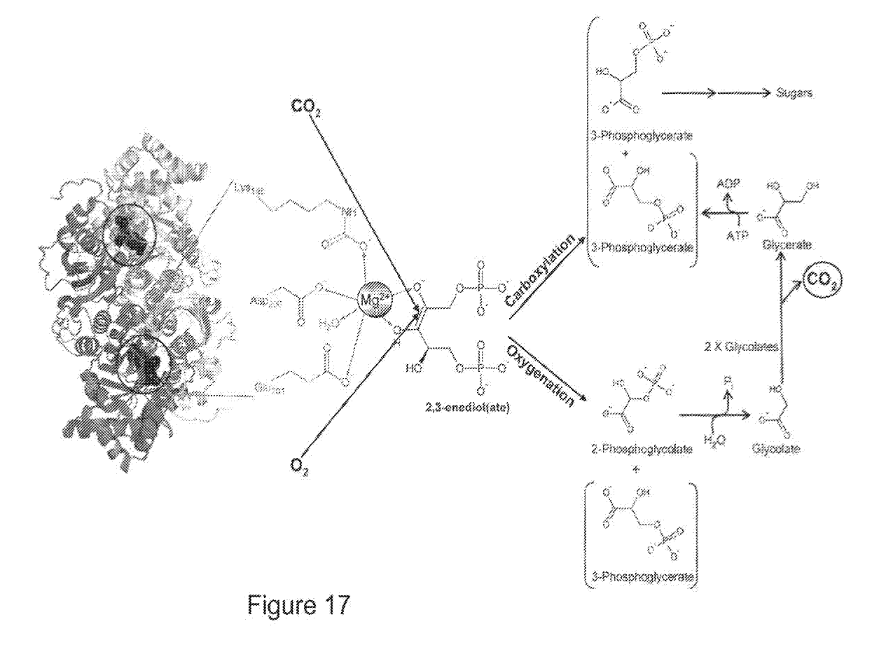

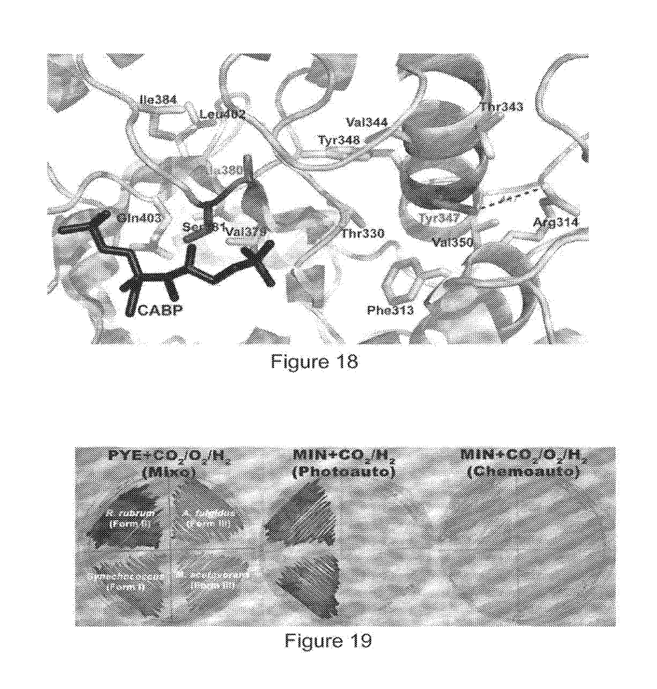

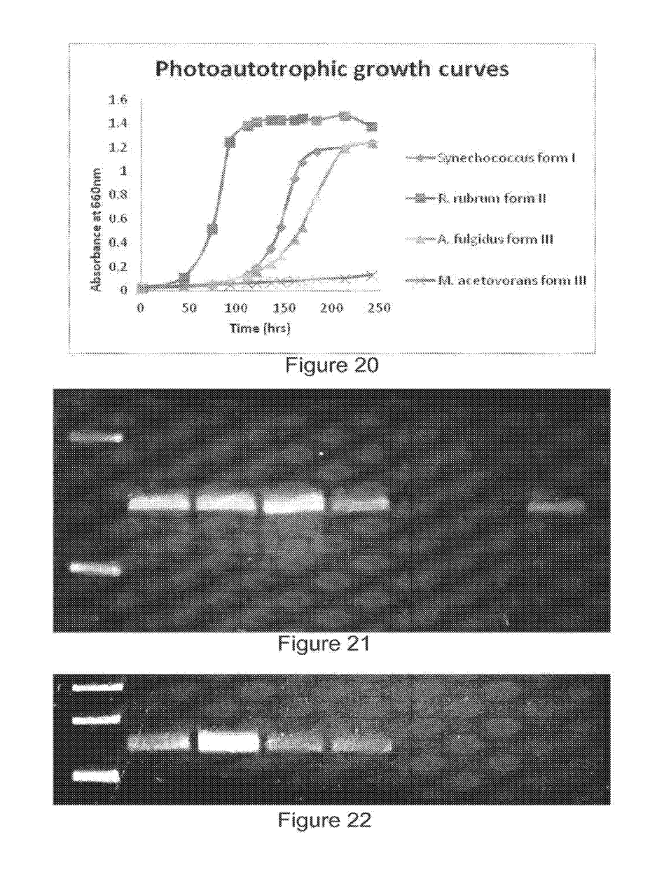

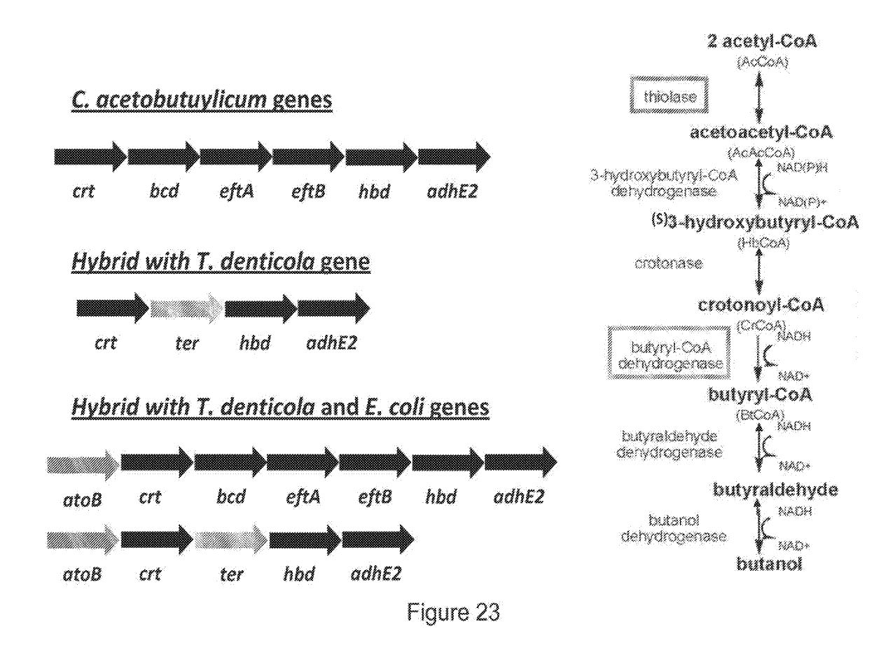

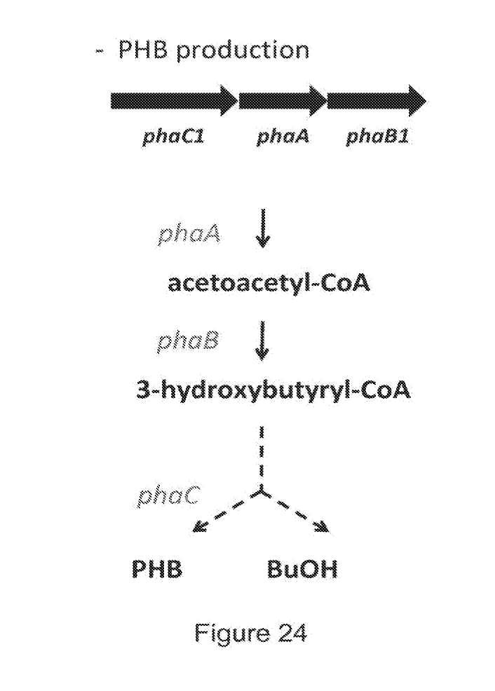

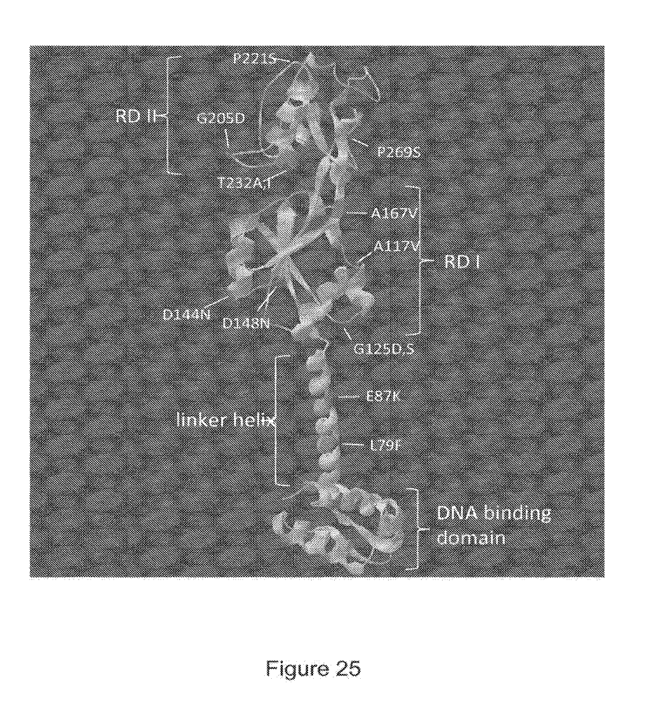

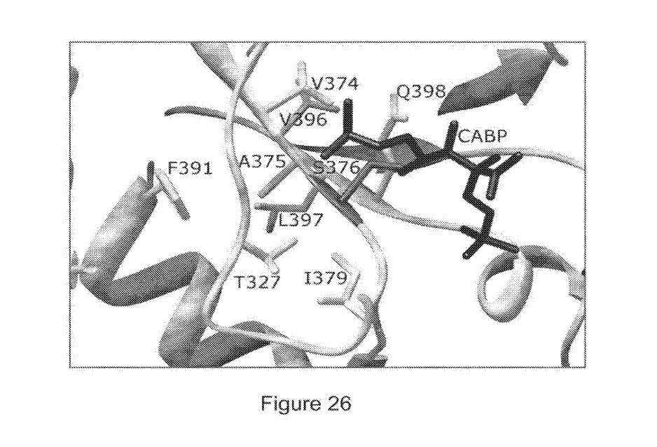

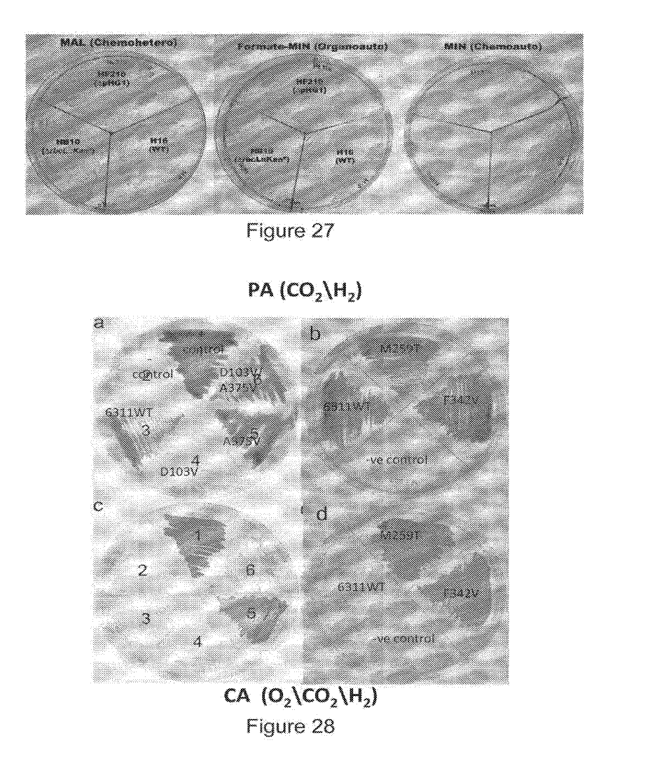

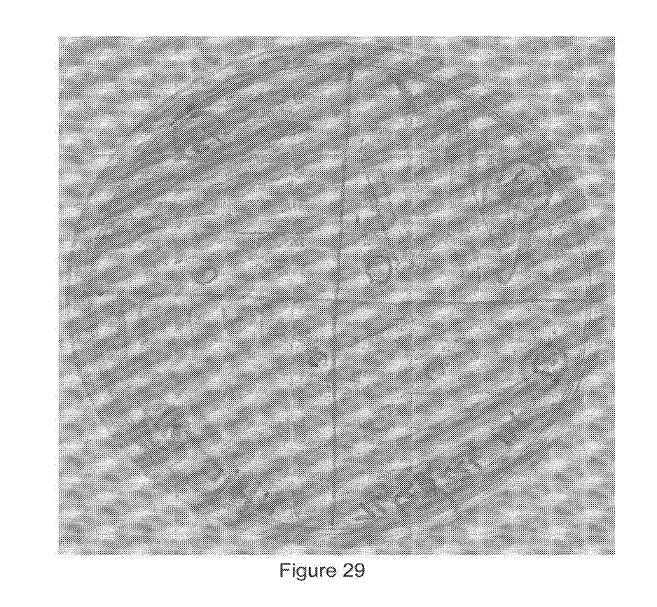

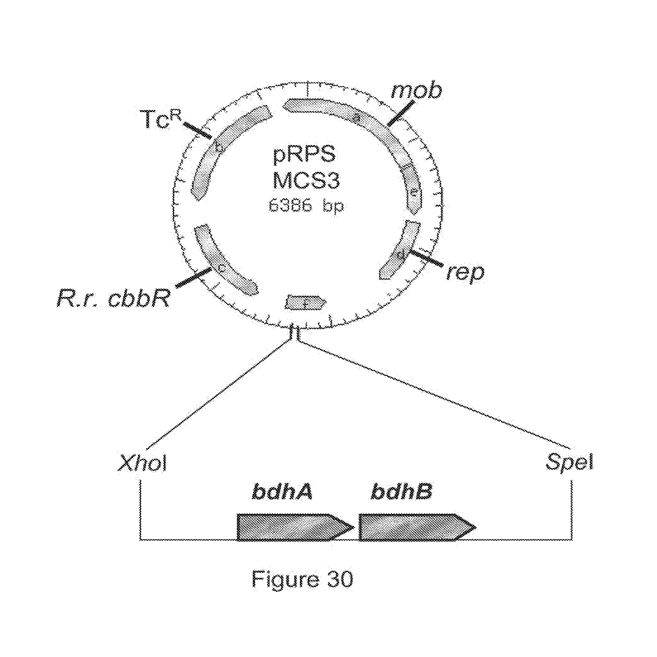

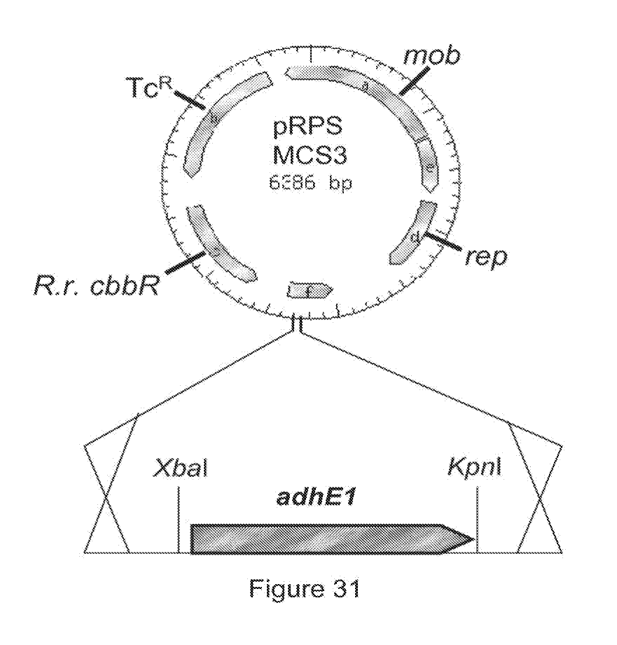

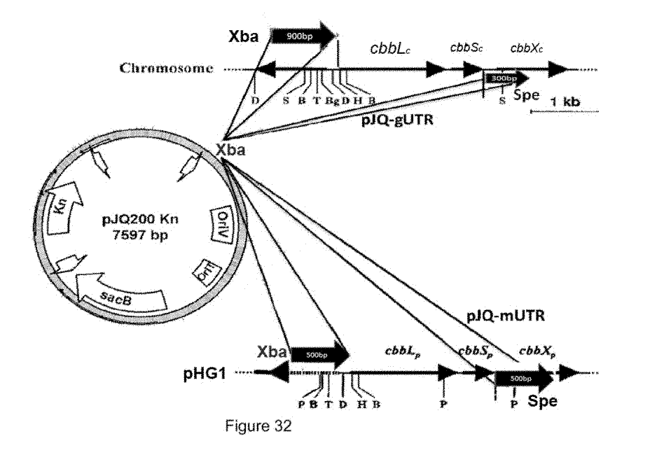

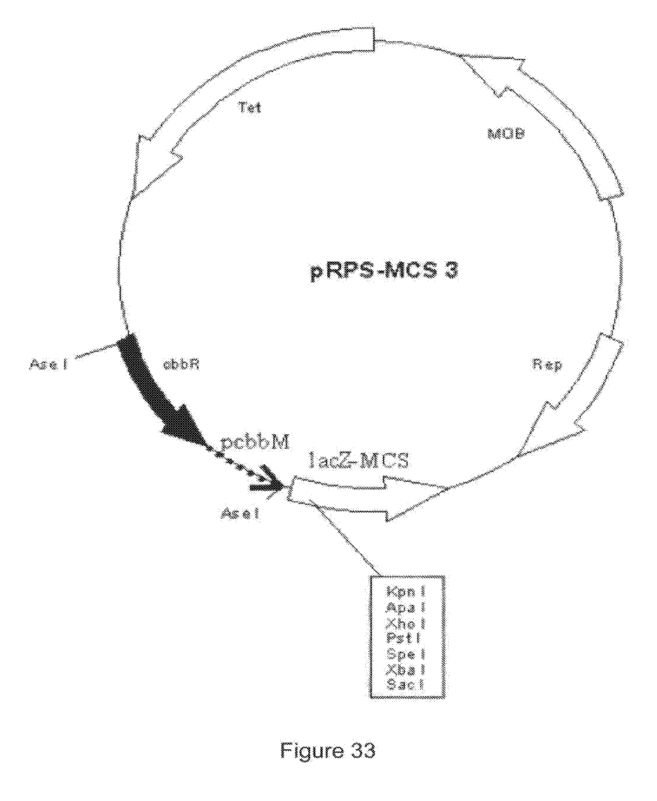

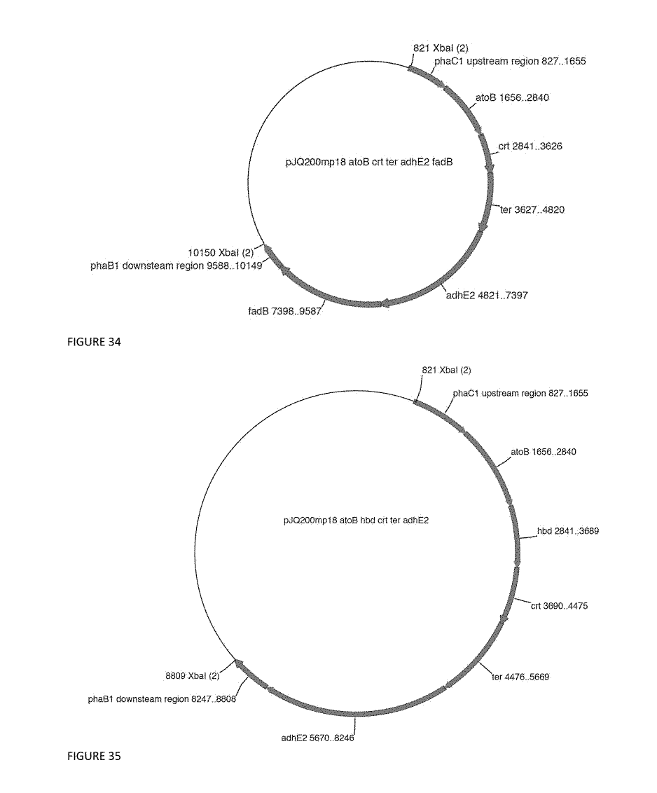

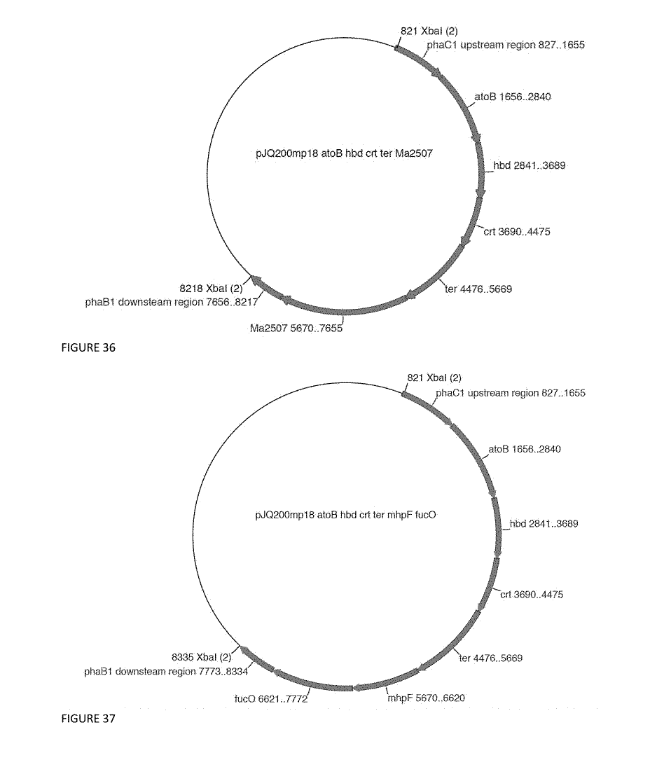

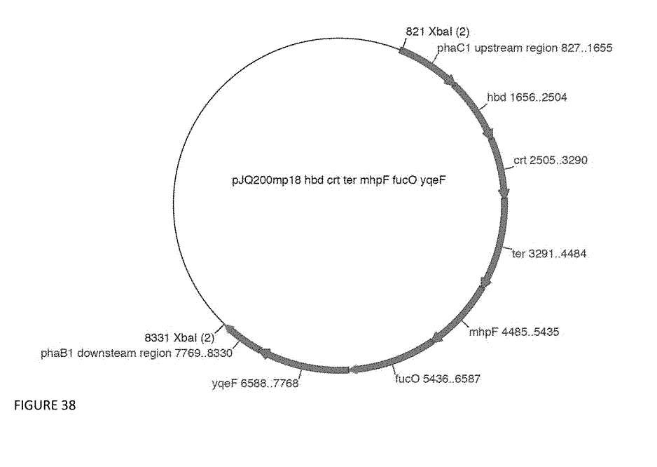

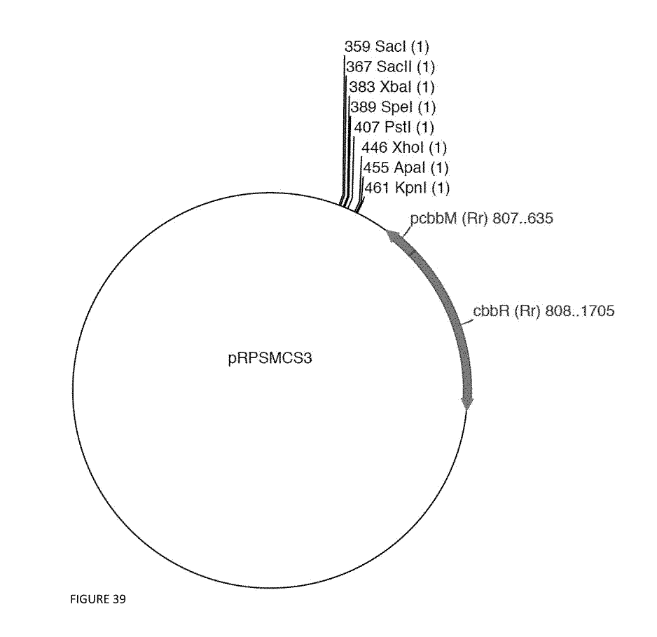

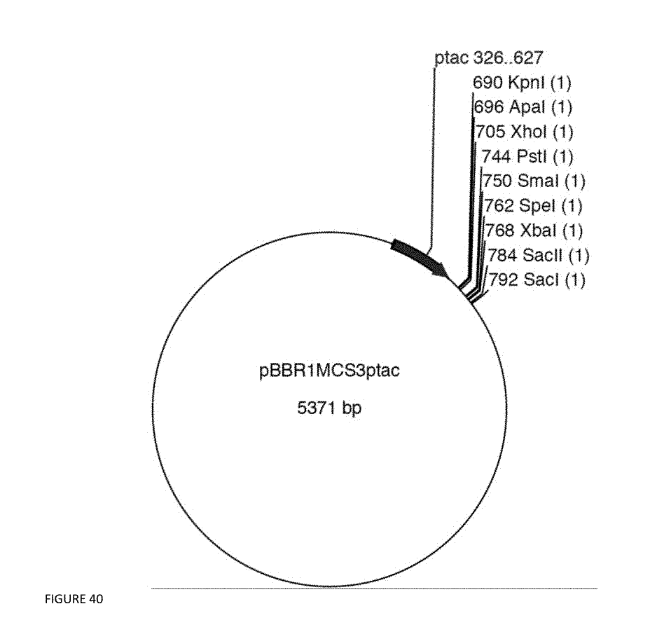

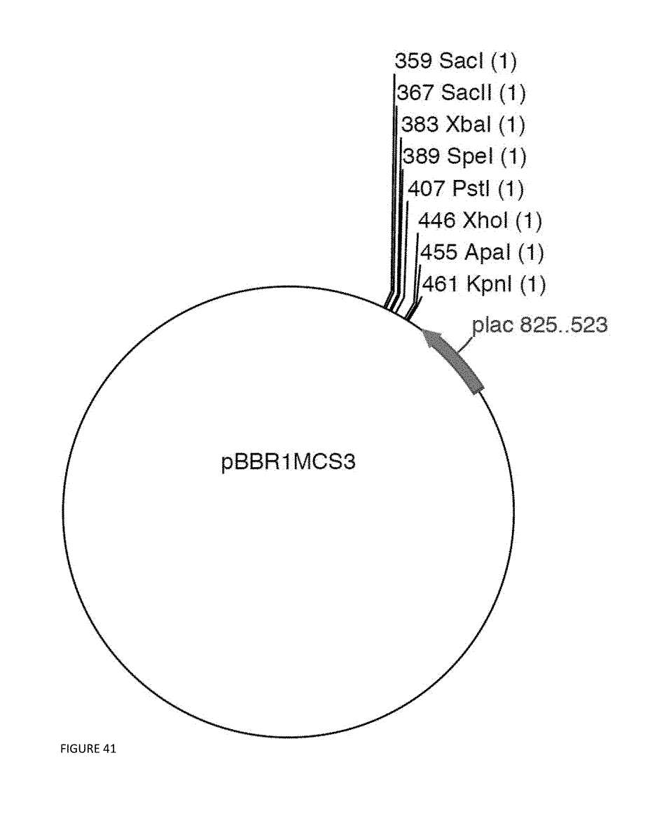

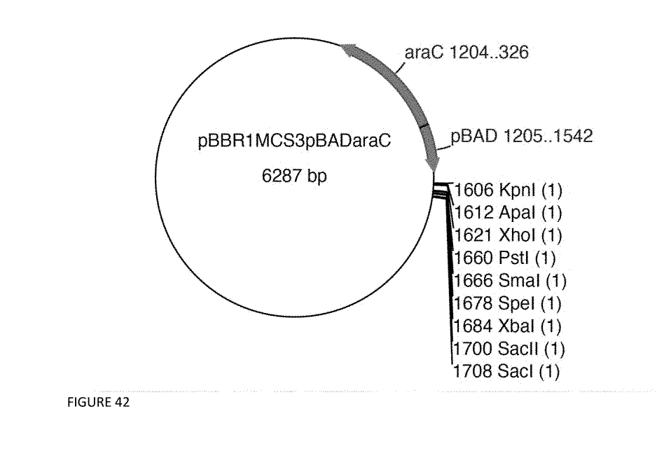

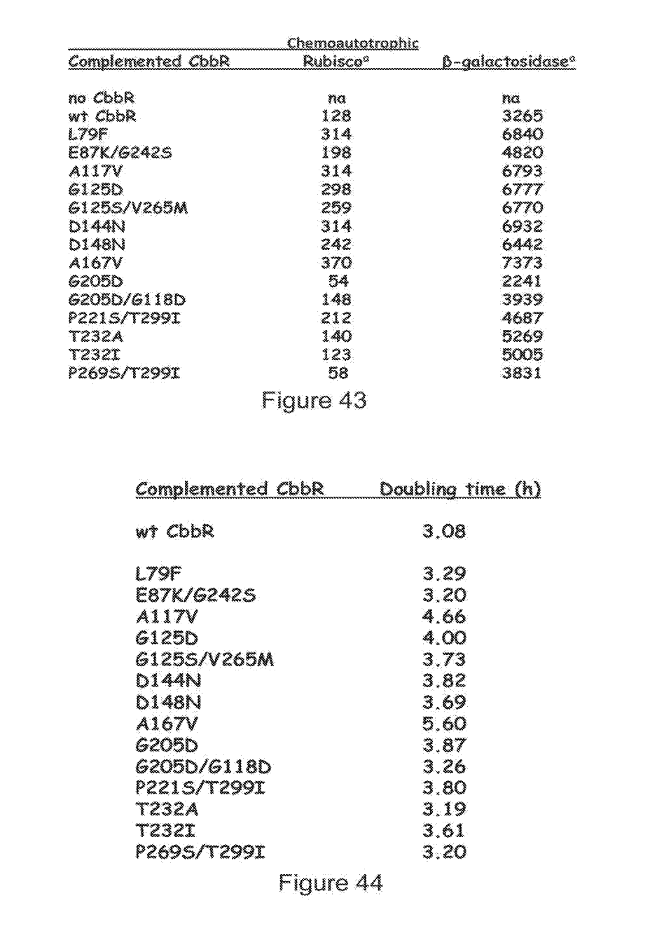

To confirm whether constitutively active mutant CbbR proteins were isolated from the putative positive selections, RubisCO and β-galactosidase activity levels were measured in strains that contained such proteins and were measured under both chemoheterotrophic and chemoautotrophic growth conditions (Table 9). Data indicate that the some mutants increased chemoheterotrophic RubisCO activities 140 to 230 fold over the levels exhibited by the controls. The data also indicated that some mutants increased chemoautotrophic RubisCO activities two fold over the levels exhibited by the controls (Table 9). Western immunoblot studies with antibodies to R. eutropha RubisCO also indicated enhanced RubisCO protein levels under these growth conditions (FIG. 14). Thus, these results illustrate that mutant CbbR proteins enhanced gene expression and increased activity levels of the rate-limiting CO₂ fixation enzyme. Table 9 shows the levels of RubisCO and β-galactosidase activity in R. eutropha H16 strains carrying mutant

TABLE 9

| | |
|---|---|
| Complemented | Chemoautotrophic |

| CbbR | Rubisco^{a} | β-galactosidase* |
|---|---|---|
| no CbbR | n/a | n/a |
| wt CbbR | 90 | 3265 |
| L79F | 209 | 6840 |
| E87K/G242S | 128 | 4312 |
| A117V | 171 | 6793 |
| G125D | 162 | 6777 |
| G125S/V265M | 162 | 6770 |
| D144N | 188 | 6932 |
| D148N | 185 | 5909 |
| A167V78 | 173 | 7373 |
| G205D | 133 | 2634 |
| P221S/T299I | 206 | 4672 |
| T232A | 78 | 4626 |
| T232I | 106 | 5005 |
| P269S/T299I | 118 | 3697 |

In Table 9, * indicates that enzyme activities are expressed in nmol/min/mg of protein under chemoautotrophic growth conditions. Values are the averages of at least three independent assays with standard deviations not exceeding 10%. In all cases, a Ralstonia eutropha cbbR gene deletion reporter strain was complemented with a CbbR constitutive mutant.

Chemoautotrophic (CO₂-dependent) growth of a cbbR knockout strain complemented with various of the mutant cbbR genes was compared to a similar construct complemented with wild-type cbbR. Under the influence of the mutant CbbR proteins, all the resultant strains showed good growth results. Many of the constitutive CbbR proteins enabled the organism to grow at a faster rate and with a shorter lag time than the strain containing the wild-type CbbR. In all cases, doubling times were better than 12 hours (Table 10). Table 10 shows the doubling times for chemoautotrophically grown Ralstonia eutropha cbbR deletion reporter strain complemented with CbbR constitutive mutants or wild type CbbR. Doubling times calculated from a log 10 scale of optical density within the exponential growth phase of cultures grown in a CO₂/H₂/O₂ atmosphere in minimal media.

TABLE 10

| Complemented CbbR | Doubling Time (h) |
|---|---|
| L79F | 5.6 |
| E87K/G242S | 6.0 |
| D144N | 6.8 |
| G205D | 7.8 |
| Wild Type | 9.9 |

With an aim to increase RubisCO's enzyme's net CO₂-fixation ability for channeling more carbon into the biosynthetic pathway for biofuel production, substitutions in the Ralstonia enzyme that would confer less sensitivity to O₂ were used. Various mutant RubisCO proteins have desired kinetic properties with respect to oxygen, while supporting good growth of R. eutropha under aerobic conditions. To directly select for improved RubisCO enzymes that are functional under oxygenic conditions, a clean RubisCO-deletion strain of Ralstonia was generated. This deletion strain can be used as the selection host (FIG. 15).

A strain of wild-type R. eutropha H16 that carries a deletion of the megaplasmid cbbLS copy was identified. PCR amplification and DNA sequencing (with multiple sets of internal and external primers) were used to confirm the genotype of the strains involved. A second construct was prepared by deleting a 984-bp region from the cbbL coding sequence that would precisely remove 328 amino acids from the RubisCO large subunit (FIG. 15). This construct, which carried only the translated regions of cbbLS, was cloned into the same suicide vector (pJQ200Km) and the clone was verified. For a discussion of suicide vector pJQ200mp18, a versatile suicide vector that allows direct selection for gene replacement, or pJQ200mp18Km, a vector with a kanamycin cassette, see Gene, 127(1): 15-21 (1993). This was mated into the megaplasmid-cbbLS deletion strain of Ralstonia. Screening for single and double-recombination resulted in a double-RubisCO deletion strain used for complementation studies.

Although “positive” mutants were identified with Synechococcus RubisCO enzymes using at least two diverse selection strategies involving R. capsulatus and E. coli hosts, none of the mutations identified resulted in an increased k_{cat} value relative to the wild type enzyme. Some of the naturally existing form II and form III RubisCO enzymes were known to have higher k_{cat} values (at the cost of higher sensitivity towards oxygen). Some of these high-k_{cat} enzymes were used with Ralstonia as a selection host to screen or directly select for randomly-introduced mutations that would result in an enzyme capable of complementation under oxygenic conditions (and thus possess decreased sensitivity for oxygen). To establish this system, the RubisCO-encoding cbbL(S) genes from Synechococcus (form I), form II (R. rubrum), and form III (A. fulgidus and M. acetovorans) were introduced in trans into strain HB10 of Ralstonia. HB10 is a megaplasmid-free strain carrying a Tn5-deletion in the genomic cbbLS genes. For discussion on HB10, see Archives of Microbiology, 154(1): 85-91 (1990)). Reintroduction of functional RubisCO genes in trans was insufficient to allow for CO₂/H₂-dependent autotrophic growth because utilization of H₂ as the energy source required the hydrogenases encoded by the genes on the megaplasmid. However, this strain could still be used for RubisCO-complementation studies using two alternative approaches.

In the first approach, complemented cells can be selected on minimal media containing format, which allows for organoautotrophic growth via the oxidation of formate to CO₂. Whereas the wild type (H16) and megaplasmid-free (HF-210) strains of Ralstonia are both capable of RubisCO-dependent autotrophic growth on formate medium, the strain HB10, which lacks RubisCO, is unable to grow. For a discussion of HF-210, see Journal of Bacteriology, 174(19): 6290-6293 (1992). Strain HB10 has been complemented with cbbL(S) genes encoding form I (Synechococcus) or form II (R. rubrum) or form III (A. fulgidus, M. acetovorans) RubisCO enzymes. These genes are able to complement for organoautotrophic growth of strain HB10. The growth is modest, which indicates that all these enzymes are expressed and functional in host HB10. Because the media gets acidified during growth on formate, the cells grow poorly on solid media. Nevertheless, O₂-pressure can be applied, and mutants of RubisCO enzymes with enhanced growth on formate medium are found.

In the second approach, growth complementation is directly assayed under CO₂/H₂-dependent chemoautotrophic conditions by complementing strain HB10 with mutant RubisCO enzymes and the genes encoding the hydrogenases responsible for H₂ oxidation on a plasmid. Various RubisCO genes are cloned into a plasmid carrying these hydrogenase genes. After verifying the constructs, the plasmids are introduced into strain HB10 to screen for oxygenic chemoautotrophic growth abilities. This system is utilized for selection of RubisCO enzymes with improved properties.

The development of n-butanol tolerance in R. eutropha H16 through previously described methods resulted in distinct isolates with various levels of resistance to this solvent. Nine isolates were identified and each of the isolates was able to grow on complex media with over 2% butanol. These isolates were named YB, X1, YB13, F5, F22, F23, F29, F51, and F52.

Six of the nine isolates were developed through the use chemostat and vapor chamber adaptation methods. The six isolates included F5, F21, F22, F23, F51, and F52. Three of the nine isolates were developed through a combination of mutagenesis and the vapor chamber adaptation method (YB, X1, and YB13; see FIG. 16 for the growth response of two such strains). Although complex media aided in the development of tolerant isolates due to increased growth rates, industrially relevant media can also be used. These isolates were grown and tested under various levels of butanol in a minimal media with CO₂ and H₂ as the carbon and energy sources, respectively. Seven isolates (of which four developed through adaptation alone and three developed through mutagenesis and adaptation) were able to grow on minimal media with CO₂ and H₂ at a level of 1.5% butanol. The seven isolates included YB, X1, YB13, F5, F23, F27, and F29. Two isolates, YB and X1, both developed solely through adaptation, were able to grow under the same conditions in the presence of 2.0% butanol. The tolerance in these two isolates represented over a six fold increase as compared the tolerance of the wild type.

### v) EXAMPLE 5

a. Engineering Metabolic Pathways of Hydrogen Bacteria for the Production of Butanol

Ralstonia eutropha produces large amounts of PHB even under conditions where CO₂ is the sole carbon source for growth. Under some growth condition, PHB synthesis may be blocked without undue hardship to the organism. Therefore, whether strains lacking the ability to synthesize PHB could funnel carbon and reducing power to desired products, such as n-butanol, was examined. The phaC1 gene was inactivated and no transcripts were produced. To prevent the production of PHB monomers, the phaC2 gene is also knocked out so that the organism cannot funnel carbon to these storage compounds. Constructs have been prepared for the construction of a dual phaC1/phaC2 knockout strain. Such a dual knockout strain preferably does not have any ability to produce PHB storage compounds.

The experiments strive to produce the maximum amount of butanol in hydrogen bacteria. These experiments adopt the following strategies: (1) the evaluation of inducible promoters for butanol gene expression, and (2) the construction and evaluation of synthetic butanol pathways.

Promoters that drive the expression of butanol related genes for increased butanol production in R. eutropha were selected. Vectors were made with the native cbbL and constitutive cbbL promoters. The cbbL promoter is native to R. eutropha and is highly expressed and regulated. The constitutive cbbL promoter was shown to increase gene expression by 2.4-fold in R. eutropha under autotrophic growth conditions. To construct strains with a constitutive cbbL promoter, the lac promoter within the pBBR1MCS-3 vector was removed and replaced by the constitutive cbbL promoter. Butanol related genes were cloned into this vector. The pBBR1MCS-3 construct was made with the native cbbL promoter.

A collection of synthetic butanol pathways were constructed in effort to increase butanol production. Five different pathways were made (Table 11). These synthetic butanol pathways were able to convert acetyl-CoA to butanol through a series of reactions. To confirm the functionality of these pathways, butanol production was evaluated in the wild-type strain BW25 113 of Escherichia coli. The production of butanol from pathways 1 (atoB, hbd, crt, ter, adhE2) and 3 (hbd, crt, ter, mhpF, fucO, yqeF) ranges from 9.0-24 mg/L. The difference in butanol production stems from what type of medium (e.g., defined or complex) was used. This butanol production test in E. coli provided positive evidence that the constructs and genes are functional. Table 11 shows a listing of synthetic BuOH pathways (See also the Figures provided herein, which provide schematic representations of these vectors).

TABLE 11

| # Construct | Syntethic BuOH Pathway |
|---|---|
| 1 | hbd, crt, ter, adhE2, atoB |
| 2 | hbd, crt, ter, mhpF, fucO, atoB |
| 3 | hbd, crt, ter, mhpF, fucO, yqeF |
| 4 | hbd, crt, ter, Ma2507, atoB |
| 5 | crt, ter, adhE2, fadB, atoB |

While the pBBR1-based vector was used to express the synthetic butanol pathway in R. eutropha, the low copy number of this plasmid hindered end-product production. To overcome this, a new gene expression vector, p3716, was created. This expression vector was produced at significantly greater copies compared to pBBR1 and gene expression could be regulated by the pBAD promoter. This promoter/vector construct was shown to enable the expression of multi-gene pathways in R. eutropha. The various BuOH pathways were subcloned from the pBBR1 vectors into the new plasmid. The pBAD promoter in p3716 replaced the native R. eutropha promoters.

b. Engineering the Metabolic Regulation of the Calvin Cycle for Constitutive Carbon Fixation Under All Growth Conditions.

The above constructs were used as starting points in mutagenesis experiments to select for enzymes that can support chemoautotrophic growth of R. capsulatus SBI/II. None of the constructs were able to support autotrophic growth. Therefore, the RubisCO genes were transferred to a different promoter/vector construct known to work in Ralstonia. (i.e., pBAD) The Ralstonia wild-type RubisCO was also cloned into a pBBR1-derived vector that carries a Ralstonia-specific “constitutive” promoter sequence. This construct was used to complement RubisCO negative strain HB10.

Constitutively active CbbR proteins, which allow high level cbb gene expression under all growth conditions, were studied. The levels of RubisCO and B-galactosidase obtained under both repressed (chemoheterotrophic or CH) and induced (chemoautotrophic or CA) growth conditions were determined. Under CH growth conditions, mutant CbbR protein G205D/R283H produced a 530 fold greater level of RubisCO than the level produced by the wild-type CbbR. The CbbR mutant E87K produced a 330 fold greater level of RubisCO than the level produced by the wild-type CbbR (Table 2). Under CA growth conditions, RubisCO levels for mutant A167V was ˜2.7 fold greater than the level for wild-type CbbR. The mutants A117V and D144N produced a 2.2 fold greater level of RubisCO than the level produced by the wild-type CbbR. RT-PCR studies confirmed these results at the level of gene expression. Table 12 shows that the Ralstonia eutropha CbbR constitutive mutants increased both expression from the cbb promoter and RuBP-dependent CO₂ fixation in vivo.

TABLE 12

| Complemented | Chemoheterotrophic | | Chemoautotrophic | |
|---|---|---|---|---|
| CbbR | RubisCO | β-galactosidase | RubisCO | β-galactosidase |
| no CbbR | 0.1 | 2 | n/a | n/a |
| wt CbbR | 0.1 | 3 | 139 | 3265 |
| H16 | 0.1 | n/a | 145 | n/a |
| (WT strain) | | | | |
| L79F | 4 | 218 | 304 | 6840 |
| E87K | 33 | 1597 | 305 | 5515 |
| E87K/G242S | 6 | 303 | 198 | 4820 |
| A117V | 6 | 254 | 314 | 6793 |
| G125D | 3 | 108 | 298 | 6777 |
| G125S/V265M | 2 | 53 | 259 | 6770 |
| D144N | 26 | 809 | 314 | 6932 |
| D148N | 8 | 343 | 242 | 6442 |
| A167V | 15 | 768 | 370 | 7373 |
| G205D | 10 | 488 | 54 | 2241 |
| G205D/G118D | 30 | 1168 | 148 | 3939 |
| G205D/R283H | 53 | 2311 | 115 | 4480 |
| P221S/T299I | 16 | 655 | 212 | 5312 |
| T232A | 4 | 212 | 140 | 5269 |
| T232I | 5 | 303 | 123 | 5005 |
| P269S/T299I | 14 | 617 | 158 | 3879 |

In Table 12, the enzyme activities are expressed in nmol/min/mg of protein. Values are averages of at least three independent assays with standard deviations not exceeding 10%. A Ralstonia eutropha cbbR gene deletion reporter strain was complemented with CbbR constitutive mutants.

Regarding the RT-PCR results, FIG. 21 shows that the CbbR mutant A117V (lane 1) has a 1.9-fold increase over the level produced by the wild type CbbR (lane 4). The CbbR mutant D144N (lane 2) has a 2.4-fold increase over level produced by the wild type CbbR (lane 4) The CbbR mutant A167V (lane 3) has a 3.3-fold increase over the level produced by the wild type CbbR (lane 4). These CbbR constitutive mutants were chosen because they had the highest RubisCO specific activities when grown in CA conditions.

A variation of the experiments shown in FIG. 21 was also performed. Here, only two constitutive CbbR mutants were used to determine whether fewer cycles of PCR would alter the reverse transcription (26 cycles for this experiment) and whether it was possible to establish a greater difference between the constitutive CbbR mutants and wild type CbbR. FIG. 22 indicates a 4.1 fold increase in transcription (for the mutant A167V) over the wild type CbbR. FIG. 22 also shows that the CbbR mutant D144N (lane 2) has a 1.8-fold increase in transcription over the wild type CbbR (lane 3). The CbbR mutant A167V (lane 3) has a 4.1-fold increase in transcription over the wild type CbbR (lane 3).These CbbR constitutive mutants were chosen because they had the highest RubisCO specific activities when grown in CA conditions

### vi) EXAMPLE 6

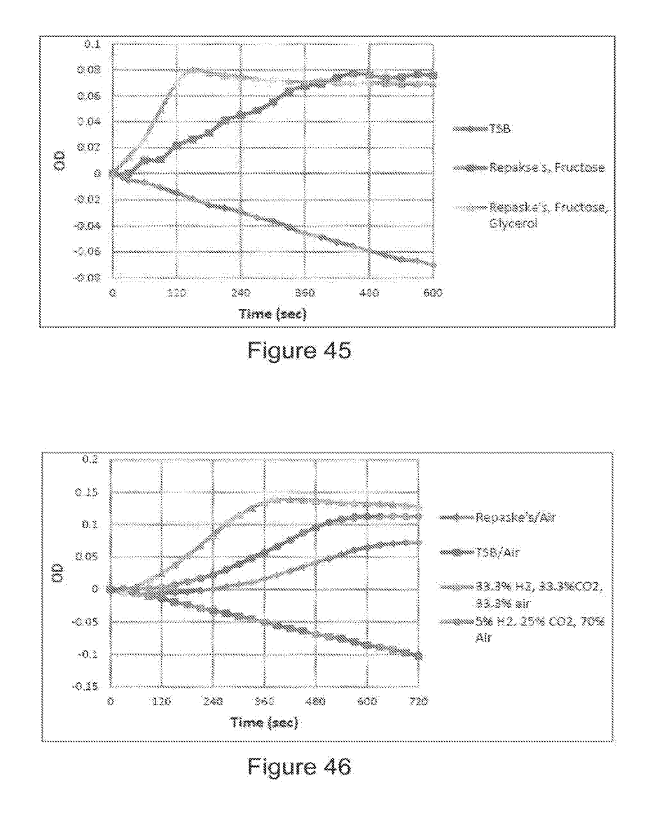

A hydrogenase enzyme activity assay was applied based on a method published by Friedrich 1981. This assay was originally performed in a cuvette but was adapted to work in a 96 well plate format to increase through-put during screening The assay measures the change in absorbance at 365 nm as NAD+is reduced to NADH by the hydrogenase enzyme. In the assay, a 0.5% solution of hexadecyltrimethyl ammonium bromide (CTAB) in hydrogen saturated 50 mM Tris was added to the well with 15 μL of bacterial culture and incubated to allow the CTAB to lyse the bacteria. Immediately prior to placing the plate into the reader, 25 μL of a 48 mM solution of NAD+ in hydrogen saturated Tris buffer was added to each well. The change in optical density was then recorded and plotted versus time. The portion of the plot showing a linear response was used to determine the rate of change that is dependent on the quantity or specific activity of the enzyme in the sample. The initial assay development work done with cultures grown on MOPS-Repaske's medium supplemented with 0.2% fructose and 0.2% glycerol showed a significant increase in enzyme activity compared to cultures grown on MOPS-Repaske's with fructose or grown in TSB (FIG. 45). This confirmed the results reported in the Friedrich paper and showed that the NAD+ was being reduced to NADH, but the results did not demonstrate that the reduction was directly related to the hydrogenase enzyme.

To prove this, R. eutropha bacteria were incubated in carbon free MOPS-Repaske's medium inside sealed serum bottles containing mixtures of H2, CO2, and air at varying ratios as shown in Table 13. R. eutropha cultures were grown overnight on TSB, pelleted, washed, and re-suspended in MOPS-Repaske's using the same volume as the initial culture to give a 1× concentrated sample. Table 13 shows the serum bottom sample matrix.

TABLE 13

| Medium | Gas Mix |
|---|---|
| TSB | 100% air |
| MOPS-Repaske's | 100% air |
| MOPS-Repaske's | 33.3% H₂, 33.3% CO₂, 33.3% air |
| MOPS-Repaske's | 5% H₂, 25% CO₂, 70% air |

Two milliliters of culture were added to 60 mL serum vials, ensuring a large ratio of head space to culture for surplus gas. The containers were sealed and 30 mL of test gas mixture was injected into each with a syringe. The vials were incubated at 30° C., and samples were taken at approximately 24 and 48 hours. Fresh gas mix was added to each vial after approximately 24 hours. As shown in FIG. 46, samples grown on TSB and air displayed no hydrogenase activity. Samples that were grown on MOPS-Repaske's with 33.3% H₂, 33.3% CO₂, and 33.3% air had greater hydrogenase enzyme activity than those grown on 5% H₂, 25% CO₂, and 70% air. Limited, but detectable enzyme activity was observed in the sample that was grown on MOPSRepaske's with 100% air, but the maximum optical density reached was much lower than the samples with mixed gases. As shown in Table 14, the hydrogenase assay showed that enzyme activity correlated well with H₂ concentrations, and the assay results were reproducible.

TABLE 14

| | Rep. 1 Rate | Rep. 2 Rate | Rep. 3 Rate |
|---|---|---|---|
| Gas | (milli-OD/min) | (milli-OD/min) | (milli-OD/min) |
| 100% air | 11.266 | 11.337 | 12.546 |
| 33.3% H₂, | 28.312 | 26.197 | 26.443 |
| 33.3% CO₂, 33.3% air | | | |
| 5% H₂, 25% CO₂, | 17.891 | 18.936 | 20.544 |
| 70% air | | | |

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### E. REFERENCES

Fukui T., Ohsawa K., Mifune J., Orita I. and Nakamura S. 2010. Evaluation of promoters for gene expression in polyhydroxyalkanoate-producing Cupriavidus necator H16. Appl Microbiol Biotechnol. Published online 29 Jan. 2011.

Shen C., Lan E., Dekishima Y., Baez A., Cho K. and Liao J. 2011. High titer anaerobic 1-butanol synthesis in Escherichia coli enabled by driving forces. Appl Environ Microbiol. Published online 11 Mar. 2011.

Khalil, A. S., and Collins, J. C. 2010. Synthetic biology: applications come of age. Nature Reviews/Genetics. 11, 367-379.

Dangel et al. (2005) Mol Microbiol 57: 1397-1414).

Dellomonaco et al. (2011) Nature.

What is claimed is:

1. A method of producing n-butanol, the method comprising:
culturing a population of aerobic hydrogen bacteria autotrophically using CO₂ as primary carbon source, the cultivation occurring in a medium,
wherein the aerobic hydrogen bacteria comprises a first genetic modification comprising one or more mutations in a gene encoding a ribulose bisphosphate carboxylase peptide, and
wherein the aerobic hydrogen bacteria comprises a second genetic modification comprising one or more mutations in a gene encoding a CbbR peptide; and
recovering the n-butanol from the medium.

2. The method of claim 1, growing successive generations and selecting the aerobic hydrogen bacteria to optimize production of the n-butanol.

3. The method of claim 2, wherein the optimization of the production of the chemical product includes optimization of the production of the n-butanol in the presence of oxygen.

4. The method of claim 3, wherein the oxygen is at a concentration of at least 5%.

5. The method of claim 1, wherein the aerobic hydrogen bacteria are cultured in the presence of oxygen, hydrogen, and carbon dioxide and in the dark.

6. The method of claims 1, wherein the mutated CbbR peptide is constitutively active.

7. The method of claim 1, wherein the aerobic hydrogen bacteria comprises a third genetic modification that inhibits or eliminates the production of polyhydroxyalkanoates by the bacteria.

8. The aerobic hydrogen bacteria of claim 1, wherein the aerobic hydrogen bacteria is Ralstonia eutropha (Cupriavidus necator), Rhodobacter capsulatus, or Rhodobacter sphaeroides.

9. The aerobic hydrogen bacteria of claims 1, wherein the aerobic hydrogen bacteria is Pseudomonas, acinomycetes, carboxidobacteria, nonsulfur purple bacteria, or purple bacteria.

10. The aerobic hydrogen bacteria of claim 1, wherein the aerobic hydrogen bacteria is Rhodospirillales, Rhizobiales Rhodospirillaceae, Rhodospirillum Acetobacteraceae, Rhodopila, Bradyrhizobiaceae, Rhodopseudomonas palustris, Hyphomicrobiaceae, Rhodomicrobium, Rhodobacteraceae, Rhodobium, Rhodobacteraceae, Rhodobacter, Rhodocyclaceae, Rhodocylus, Comamonadaceae, Cupriavidus, or Rhodoferax.

11. The method of claim 1, wherein the mutations in a gene encoding for ribulose bisphosphate carboxylase peptide results in an increase in efficiency of the peptide to fix CO₂ at a fixed oxygen concentration.

12. The method of claim 11, wherein the mutations in a gene encoding for ribulose bisphosphate carboxylase peptide result in a decrease of the sensitivity of the CO₂ fixation efficiency to O₂ at a fixed CO₂ concentration.

13. The method of claim 1, wherein the mutated ribulose bisphosphate carboxylase peptide comprises a mutation that results in a codon change, wherein the codon change is a change from GGC to GGT at position 264, from TCG to ACC at position 265, from GAC to GAT at position 271, from GTG to GGC at position 274, from TAC to GTC at position 347, from GCC to GTC at position 380, or a combination thereof.

14. The method of claim 1, wherein the mutated CbbR peptide comprises a mutation, wherein the mutation is L79F, E87K, E87K/G242S, G98R, A117V, G125D, G125S/V265M, D144N, D148N, A167V, G205D, G205S, G205D/G118D, G205D/R283H, P221 S, P221S/T299I, T232A, T232I, P269S, P269S/T299I, R272Q, G80D/S106N/G261E, or a combination thereof.

15. The method of claim 1, wherein the aerobic hydrogen bacteria further comprises one or more endogenous genes that is silenced or knocked out, wherein the one or more genes that is silenced or knocked out encode a peptide capable of converting (i) acetyl-CoA to acetoacetyl-CoA, (ii) acetoacetyl-CoA to β-hydroxybutyryl-CoA, or (iii) β-hydroxybutyryl-CoA to polyhydroxyalkanoate.

16. The method of claim 15, wherein the one or more endogenous genes silenced or knocked out is selected from the group consisting of phaA, phaB1, phaC1, and phaC2.


SEQUENCE LISTING (46 sequences)

SEQ ID NO: 1 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 2 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRIFGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 3 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEKGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 4 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEKGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMSISLLSLHTLGLELRTGEIGLLDVAGTPIERIWHVAHMSSKRLSPASESCRAYLLEHTAEFLGREYGGLMPGRRVA

SEQ ID NO: 5 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQRSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 6 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLVGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 7 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPDVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 8 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPSVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDMAGTPIERIWHVAHMSSKRLSPASESCRAYLLEHTAEFLGREYGGLMPGRRVA

SEQ ID NO: 9 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQNNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 10 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAINLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 11 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIVAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 12 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSDTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 13 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLADFTALHPGVDLRIAEGNRETLLRLLQDNAIDLALMGRPPRELDAVSEPIAAHPHVLVASPRHPLHDAKGFDLQELRHETFLLREPGSDTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 14 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSDTRTVAEYMFRDHLFTPAKVITLGSNETIKQAVMAGMGISLLSLHTLGLELRTGEIGLLDVAGTPIERIWHVAHMSSKHLSPASESCRAYLLEHTAEFLGREYGGLMPGRRVA

SEQ ID NO: 15 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTSAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 16 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTSAKVITLGSNETIKQAVMAGMGISLLSLHTLGLELRTGEIGLLDVAGTPIERIWHVAHMSSKRLSPASESCRAYLLEHIAEFLGREYGGLMPGRRVA

SEQ ID NO: 17 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNEAIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 18 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNEIIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 19 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTSIERIWHVAHMSSKRLSPASESCRAYLLEHTAEFLGREYGGLMPGRRVA

SEQ ID NO: 20 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTSIERIWHVAHMSSKRLSPASESCRAYLLEHIAEFLGREYGGLMPGRRVA

SEQ ID NO: 21 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIGLLDVAGTPIEQIWHVAHMSSKRLSPASESCRAYLLEHTAEFLGREYGGLMPGRRVA

SEQ ID NO: 22 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILDEVKDAEEGLQAVKDVEQGSITIGLINTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSGTRTVAEYMFRDHLFTPAKVITLGSNETIKQAVMAGMGISLLSLHTLGLELRTGEIELLDVAGTPIERIWHVAHMSSKRLSPASESCRAYLLEHTAEFLGREYGGLMPGRRVA

SEQ ID NO: 23 - protein, 317 aa, Artificial Sequence
  Description of Artificial Sequence; note = synthetic construct
MSSFLRALTLRQLQIFVTVARHASFVRAAEELHLTQPAVSMQVKQLESVVGMALFERVKGQLTLTEPGDRLLHHASRILGEVKDAEEGLQAVKDVEQGSITIGLISTSKYFAPKLLAGFTALHPGVDLRIAEGNRETLLRLLQDNAIDLALMGRPPRELDAVSEPIAAHPHVLVASPRHPLHDAKGFDLQELRHETFLLREPGSSTRTVAEYMFRDHLFTPAKVITLGSNETIKQAVMAGMGISLLSLHTLGLELRTGEIGLLDVAGTPIERIWHVAHMSSKRLSPASESCRAYLLEHTAEFLGREYGGLMPGRRVA

SEQ ID NO: 24 - protein, 486 aa, Artificial Sequence
  Description of Artificial Sequence; note = synthetic construct
MNAPESVQAKPRKRYDAGVMKYKEMGYWDGDYEPKDTDLLALFRITPQDGVDPVEAAAAVAGESSTATWTVVWTDRLTACDMYRAKAYRVDPVPNNPEQFFCYVAYDLSLFEEGSIANLTASIIGNVFSFKPIKAARLEDMRFPVAYVKTFAGPSTGIIVERERLDKFGRPLLGATTKPKLGLSGRNYGRVVYEGLKGGLDFMKDDENINSQPFMHWRDRFLFVMDAVNKASAATGEVKGSYLNVTAGTMEEMYRRAEFAKSLGSVVIMIDLIVGWTCIQSMSNWCRQNDMILHLHRAGHGTYTRQKNHGVSFRVIAKWLRLAGVDHMHTGTAVGKLEGDPLTVQGYYNVCRDAYTHTDLTRGLFFDQDWASLRKVMPVASGGIHAGQMHQLIHLFGDDVVLQFGGGTIGHPQGIQAGATANRVALEAMVLARNEGRDILNEGPEILRDAARWCGPLRAALDTWGDISFNYTPTDTSDFAPTASVA

SEQ ID NO: 25 - protein, 486 aa, Artificial Sequence
  Description of Artificial Sequence; note = synthetic construct
MNAPESVQAKPRKRYDAGVMKYKEMGYWDGDYEPKDTDLLALFRITPQDGVDPVEAAAAVAGESSTATWTVVWTDRLTACDMYRAKAYRVDPVPNNPEQFFCYVAYDLSLFEEGSIANLTASIIGNVFSFKPIKAARLEDMRFPVAYVKTFAGPSTGIIVERERLDKFGRPLLGATTKPKLGLSGRNYGRVVYEGLKGGLDFMKDDENINSQPFMHWRDRFLFVMDAVNKASAATGEVKGSYLNVTAGTMEEMYRRAEFAKSLGTVVIMIDLIVGWTCIQSMSNWCRQNDMILHLHRAGHGTYTRQKNHGVSFRVIAKWLRLAGVDHMHTGTAVGKLEGDPLTVQGYYNVCRDAYTHTDLTRGLFFDQDWASLRKVMPVASGGIHAGQMHQLIHLFGDDVVLQFGGGTIGHPQGIQAGATANRVALEAMVLARNEGRDILNEGPEILRDAARWCGPLRAALDTWGDISFNYTPTDTSDFAPTASVA

SEQ ID NO: 26 - protein, 486 aa, Artificial Sequence
  Description of Artificial Sequence; note = synthetic construct
MNAPESVQAKPRKRYDAGVMKYKEMGYWDGDYEPKDTDLLALFRITPQDGVDPVEAAAAVAGESSTATWTVVWTDRLTACDMYRAKAYRVDPVPNNPEQFFCYVAYDLSLFEEGSIANLTASIIGNVFSFKPIKAARLEDMRFPVAYVKTFAGPSTGIIVERERLDKFGRPLLGATTKPKLGLSGRNYGRVVYEGLKGGLDFMKDDENINSQPFMHWRDRFLFVMDAVNKASAATGEVKGSYLNVTAGTMEEMYRRAEFAKSLGSVVIMIDLIGGWTCIQSMSNWCRQNDMILHLHRAGHGTYTRQKNHGVSFRVIAKWLRLAGVDHMHTGTAVGKLEGDPLTVQGYYNVCRDAYTHTDLTRGLFFDQDWASLRKVMPVASGGIHAGQMHQLIHLFGDDVVLQFGGGTIGHPQGIQAGATANRVALEAMVLARNEGRDILNEGPEILRDAARWCGPLRAALDTWGDISFNYTPTDTSDFAPTASVA

SEQ ID NO: 27 - protein, 486 aa, Artificial Sequence
  Description of Artificial Sequence; note = synthetic construct
MNAPESVQAKPRKRYDAGVMKYKEMGYWDGDYEPKDTDLLALFRITPQDGVDPVEAAAAVAGESSTATWTVVWTDRLTACDMYRAKAYRVDPVPNNPEQFFCYVAYDLSLFEEGSIANLTASIIGNVFSFKPIKAARLEDMRFPVAYVKTFAGPSTGIIVERERLDKFGRPLLGATTKPKLGLSGRNYGRVVYEGLKGGLDFMKDDENINSQPFMHWRDRFLFVMDAVNKASAATGEVKGSYLNVTAGTMEEMYRRAEFAKSLGSVVIMIDLIVGWTCIQSMSNWCRQNDMILHLHRAGHGTYTRQKNHGVSFRVIAKWLRLAGVDHMHTGTAVGKLEGDPLTVQGVYNVCRDAYTHTDLTRGLFFDQDWASLRKVMPVASGGIHAGQMHQLIHLFGDDVVLQFGGGTIGHPQGIQAGATANRVALEAMVLARNEGRDILNEGPEILRDAARWCGPLRAALDTWGDISFNYTPTDTSDFAPTASVA

SEQ ID NO: 28 - protein, 486 aa, Artificial Sequence
  Description of Artificial Sequence; note = synthetic construct
MNAPESVQAKPRKRYDAGVMKYKEMGYWDGDYEPKDTDLLALFRITPQDGVDPVEAAAAVAGESSTATWTVVWTDRLTACDMYRAKAYRVDPVPNNPEQFFCYVAYDLSLFEEGSIANLTASIIGNVFSFKPIKAARLEDMRFPVAYVKTFAGPSTGIIVERERLDKFGRPLLGATTKPKLGLSGRNYGRVVYEGLKGGLDFMKDDENINSQPFMHWRDRFLFVMDAVNKASAATGEVKGSYLNVTAGTMEEMYRRAEFAKSLGSVVIMIDLIVGWTCIQSMSNWCRQNDMILHLHRAGHGTYTRQKNHGVSFRVIAKWLRLAGVDHMHTGTAVGKLEGDPLTVQGYYNVCRDAYTHTDLTRGLFFDQDWASLRKVMPVVSGGIHAGQMHQLIHLFGDDVVLQFGGGTIGHPQGIQAGATANRVALEAMVLARNEGRDILNEGPEILRDAARWCGPLRAALDTWGDISFNYTPTDTSDFAPTASVA

SEQ ID NO: 29 - DNA, 207 bp, Artificial Sequence
  Description of Artificial Sequence; note = synthetic construct
gcaactggcgaagggtaagggcgcgcaggaaggacgacatgggcggttgggggcggctttggatggtcccgtgatgtgcagcttggtccgcacttaagggattgcttatacaggggctaagaatatctgaatttaccttatgtgggtgggcttatatctttgcatcaacgcagcagccaagacgctcaaccacgcaaggagacaagc

SEQ ID NO: 30 - DNA, 207 bp, Artificial Sequence
  Description of Artificial Sequence; note = synthetic construct
gcaactggcgaagggtaagggcgcgcaggaaggacgacatgggcggttgggggcggctttggatggtcccgtgatgtgcagcttggtccgcacttaagggattgcttatacaggggctaagaatatctgaattgacattatgtgggtgggcttatataattgcatcaacgcagcagccaagacgctcaaccacgcaaggagacaagc

SEQ ID NO: 31 - DNA, 122 bp, Artificial Sequence
  Description of Artificial Sequence; note = synthetic construct
gcgcaacgcaattaatgtgagttagctcactcattaggcaccccaggctttacactttatgcttccggctcgtatgttgtgtggaattgtgagcggataacaatttcacacaggaaacagct

SEQ ID NO: 32 - DNA, 311 bp, Artificial Sequence
  Description of Artificial Sequence; note = synthetic construct
cgcaacgcaattaatgtaagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacag

SEQ ID NO: 33 - DNA, 447 bp, Artificial Sequence
  Description of Artificial Sequence; note = synthetic construct
caaaaattcatccttctcgcctatgctctggggcctcggcagatgcgagcgctgcataccgtccggtaggtcgggaagcgtgcagtgccgaggcggattcccgcattgacagcgcgtgcgttgcaaggcaacaatggactcaaatgtctcggaatcgctgacgattcccaggtttctccggcaagcatagcgcatggcgtctccatgcgagaatgtcgcgcttgccggataaaaggggagccgctatcggaatggacgcaagccacggccgcagcaggtgcggtcgagggcttccagccagttccagggcagatgtgccggcagaccctcccgctttgggggaggcgcaagccgggtccattcggatagcatctccccatgcaaagtgccggccagggcaatgcccggagccggttcgaatagtgacggcagagagacaatcaaatc

SEQ ID NO: 34 - DNA, 173 bp, Artificial Sequence
  Description of Artificial Sequence; note = synthetic construct
gcgacgccatccgcaccctgccgccgcgccgcaaccgtcatgtcagcggctgaaaagcgcggacaacggaaagtcgtataatcttttacttatggggaagtctaaaacaataaattatggcttatggatcgatgggggtacagtgccccccatcgaacatctagggagagtcc

SEQ ID NO: 35 - DNA, 344 bp, Artificial Sequence
  Description of Artificial Sequence; note = synthetic construct
acttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagctaaggaggagacc

SEQ ID NO: 36 - DNA, 6387 bp, Artificial Sequence
  Description of Artificial Sequence; note = synthetic construct
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaatggactctccctagatgttcgatggggggcactgtacccccatcgatccataagccataatttattgttttagacttccccataagtaaaagattatacgactttccgttgtccgcgcttttcagccgctgacatgacggttgcggcgcggcggcagggtgcggatggcgtcgctcaacaggtcctcgccgcgcgccttgagaaaggccaccatcgcctcggccaccggcagcaggtgcttgccctcgcggatcaccacataccagtcgcgctggatcggcagcccttccacatcaaggatcaccagccggccgaccgaaagctccaggctcatggtgttgcgcgacagcaggctgatgcccatgccggccatcaccgcctgcttgatggtctcattgctcgacatctcgatcatgcggtggggcagcaccccatggtcggtcatcagcttttccataaggatgcgcgtgcccgaccccggctcgcgcatcagaaaggtttcccccgacagatcgtggaacgtcagcttgcgccgcaccagatgatccgacgccgcgaccatcaccatcggattgggggcgagttcggcgcgcaccgccggctcggtcggcggccggcccatgatgaacaggtccagggcgttttcctggatcatccccaggatctgctcgcgattggccaccgtcagccccagttcaacaccgggatagccggcggtgaacaccgagagcaggcggggggcgaagtatttggcggtgctgaccacgccgatgcgcaacgccccggcgcgcttgcccttcagggcgtccatcgccttgtcggcatcggtcaccgccgccagaatggtccgcacatgcccgagaagaatggttccggcctgggtgagcagcagcacccgacccatctgctcaaacagcggcaagccggccagggcctcgatctgcttgatttgcaacgacaccgccggctgggtcagccccagttcccgggcggcgttggagaagctgaggtggcgggcgacggcgtcgaaaatctgcatctgccgcaagtggcgtggcgcatggcggatcattcccctgccgattggcctataaggtttagcttatagactatgccataataactttgttgtgtttatgtgtccgtcccgccagaatttccatggtggatttaggggttcacaaggccccaacccctcccacccatcaggagaattaatgaatcggccaacgcgcggggagaggcggtttgcgtattgggcgcatttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt

SEQ ID NO: 37 - DNA, 1197 bp, Artificial Sequence
  Description of Artificial Sequence; note = synthetic construct
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggccgagggcaaggccgaggccaccggtccgctgcacgaccggcgcttcgccggcgacgcatggcgcaccaacctcccatatcgcttcgctgccgcgttctacctgctcaatgcgcgcgccttgaccgagctggccgatgccgtcgaggccgatgccaagacccgccagcgcatccgcttcgcgatctcgcaatgggtcgatgcgatgtcgcccgccaacttccttgccaccaatcccgaggcgcagcgcctgctgatcgagtcgggcggcgaatcgctgcgtgccggcgtgcgcaacatgatggaagacctgacacgcggcaagatctcgcagaccgacgagagcgcgtttgaggtcggccgcaatgtcgcggtgaccgaaggcgccgtggtcttcgagaacgagtacttccagctgttgcagtacaagccgctgaccgacaaggtgcacgcgcgcccgctgctgatggtgccgccgtgcatcaacaagtactacatcctggacctgcagaacgagctcaaggtaccgggcaagctgaccgtgtgcggcgtgccggtggacctggccagcatcgacgtgccgacctatatctacggctcgcgcgaagaccatatcgtgccgtggaccgcggcctatgcctcgaccgcgctgctggcgaacaagctgcgcttcgtgctgggtgcgtcgggccatatcgccggtgtgatcaacccgccggccaagaacaagcgcagccactggactaacgatgcgctgccggagtcgccgcagcaatggctggccggcgccatcgagcatcacggcagctggtggccggactggaccgcatggctggccgggcaggccggcgcgaaacgcgccgcgcccgccaactatggcaatgcgcgctatcgcgcaatcgaacccgcgcctgggcgatacgtcaaagccaaggcatga

SEQ ID NO: 38 - DNA, 504 bp, Artificial Sequence
  Description of Artificial Sequence; note = synthetic construct
atggcgaccggcaaaggcgcggcagcttccacgcaggaaggcaagtcccaaccattcaaggtcacgccggggccattcgatccagccacatggctggaatggtcccgccagtggcagggcactgaaggcaacggccacgcggccgcgtccggcattccgggcctggatgcgctggcaggcgtcaagatcgcgccggcgcagctgggtgatatccagcagcgctacatgaaggacttctcagcgctgtggcaggccatggcactggcgcaggaagtggcgaccaagggcgtgaccgtcaacacggtctctccgggctatatcgccaccgacatggtcaaggcgatccgccaggacgtgctcgacaagatcgtcgcgacgatcccggtcaagcgcctgggcctgccggaagagatcgcctcgatctgcgcctggttgtcgtcggaggagtccggtttctcgaccggcgccgacttctcgctcaacggcggcctgcatatgggctga

SEQ ID NO: 39 - DNA, 1197 bp, Artificial Sequence
  Description of Artificial Sequence; note = synthetic construct
gtgaacgccaagcatgagaagtaccagcgcctgattgattactgcaaggccatgccgcctacaccgaccgcggtggcgcatccgtgcgaccagtcttcgctggaaggcgccgtagaggccgcccggctgggcctgatcgcgccgatcctggttgggccgcgttcccgcatcgaggacgccgcgcgcgcggccggcattgacatccgcgagtacccgattgtcgatgccgagcacagccatgcggcggcggctgccgcagtgcaactggtgcgcgaaagcaaggcagaggctctgatgaagggcagtctgcacaccgatgagctgatgggagccgtggtcgcgggtaacagcggcttgcgcaccggccggcgcatcagccactgcttcgtgatggatgtgcccggccacgaggacgctctgatcatcaccgacgctgccgtcaatattgccccgacgcttgccgagaaggccggcatcctgcaaaacgcgatcgacctggcccatgccttgcaggtcaaggaggtccgccttcatcagtcatgcacccacggactgtcctatgagtacatcgccagtgtcctcccgagcgttgatgcgggtgcagcggcgggccgcacgatcgtggcccacctcggcaacggcagcagcatgtgtgcgctggtggcggggcgcagcgtggccagcacgatgggcttcactgcggtggatggcctgccgatgggaactcgctgcggcagcctcgatccgggcgtcatcctctacctgatcagcgaactcggcatggatgcccgcgccatcgaggacctgatctatcgaaaatccggtctgcttggcgtctccggcctgtcgagcgacatgcgcgcgctgctcgccagcgacgatgtgcaggcccgttttgccgtcgaactgtacacgtaccgcgtcgcccgggagcttggttcgctggccgccgccgcacaggggctggacgcgctggtcttcaccgctggcatcggcgagcatgccgcgccgatccgcgagcgcgtatgccggctggcggcatggctgggggtgagtgtcgatcccgcggcgaacgccagcgacggaccgcgcatcagcttagcctcgggcaatgtcccggtctgggtcatcccgaccaacgaggaactgatgattgccaggcatacccgggaggtcctggcggcacccgctcgatga

SEQ ID NO: 40 - DNA, 8316 bp, Artificial Sequence
  Description of Artificial Sequence; note = synthetic construct
acatggtactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagctaaggaggagaccccatgggagagctcggtacccggggatcctctagagtcgacctgcaggcatgcaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 41 - DNA, 7684 bp, Artificial Sequence
  Description of Artificial Sequence; note = synthetic construct
acatggtactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgggcaactggcgaagggtaagggcgcgcaggaaggacgacatgggcggttgggggcggctttggatggtcccgtgatgtgcagcttggtccgcacttaagggattgcttatacaggggctaagaatatctgaatttaccttatgtgggtgggcttatatctttgcatcaacgcagcagccaagacgctcaaccacgcaaggagacaagcgagctcggtacccggggatcctctagagtcgacctgcaggcatgcaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 42 - DNA, 7684 bp, Artificial Sequence
  Description of Artificial Sequence; note = synthetic construct
acatggtactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgggcaactggcgaagggtaagggcgcgcaggaaggacgacatgggcggttgggggcggctttggatggtcccgtgatgtgcagcttggtccgcacttaagggattgcttatacaggggctaagaatatctgaattgacattatgtgggtgggcttatatctttgcatcaacgcagcagccaagacgctcaaccacgcaaggagacaagcgagctcggtacccggggatcctctagagtcgacctgcaggcatgcaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 43 - DNA, 5371 bp, Artificial Sequence
  Description of Artificial Sequence; note = synthetic construct
gcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaatgcgcccaatacgcaaaccgcctctccccgcgcgttggccgattcattatgcgcaacgcaattaatgtaagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacattatgatgaccatgattacgccaagcgcgcaattaaccctcactaaagggaacaaaagctgggtaccgggccccccctcgaggtcgacggtatcgataagcttgatatcgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggaca

SEQ ID NO: 44 - DNA, 6287 bp, Artificial Sequence
  Description of Artificial Sequence; note = synthetic construct
gcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaatgcgcccaatacgcaaaccgcctctccccgcgcgttggccgattcattaatttatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttggatggagtgaaacgattaatgatgaccatgattacgccaagcgcgcaattaaccctcactaaagggaacaaaagctgggtaccgggccccccctcgaggtcgacggtatcgataagcttgatatcgaattcctgcagcccgggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggtgaatccgttagcgaggtgccgccggcttccattcaggtcgaggtggcccggctccatgcaccgcgacgcaacgcggggaggcagacaaggtatagggcggcgaggcggctacagccgatagtctggaacagcgcacttacgggttgctgcgcaacccaagtgctaccggcgcggcagcgtgacccgtgtcggcggctccaacggctcgccatcgtccagaaaacacggctcatcgggcatcggcaggcgctgctgcccgcgccgttcccattcctccgtttcggtcaaggctggcaggtctggttccatgcccggaatgccgggctggctgggcggctcctcgccggggccggtcggtagttgctgctcgcccggatacagggtcgggatgcggcgcaggtcgccatgccccaacagcgattcgtcctggtcgtcgtgatcaaccaccacggcggcactgaacaccgacaggcgcaactggtcgcggggctggccccacgccacgcggtcattgaccacgtaggccgacacggtgccggggccgttgagcttcacgacggagatccagcgctcggccaccaagtccttgactgcgtattggaccgtccgcaaagaacgtccgatgagcttggaaagtgtcttctggctgaccaccacggcgttctggtggcccatctgcgccacgaggtgatgcagcagcattgccgccgtgggtttcctcgcaataagcccggcccacgcctcatgcgctttgcgttccgtttgcacccagtgaccgggcttgttcttggcttgaatgccgatttctctggactgcgtggccatgcttatctccatgcggtagggtgccgcacggttgcggcaccatgcgcaatcagctgcaacttttcggcagcgcgacaacaattatgcgttgcgtaaaagtggcagtcaattacagattttctttaacctacgcaatgagctattgcggggggtgccgcaatgagctgttgcgtaccccccttttttaagttgttgatttttaagtctttcgcatttcgccctatatctagttctttggtgcccaaagaagggcacccctgcggggttcccccacgccttcggcgcggctccccctccggcaaaaagtggcccctccggggcttgttgatcgactgcgcggccttcggccttgcccaaggtggcgctgcccccttggaacccccgcactcgccgccgtgaggctcggggggcaggcgggcgggcttcgccttcgactgcccccactcgcataggcttgggtcgttccaggcgcgtcaaggccaagccgctgcgcggtcgctgcgcgagccttgacccgccttccacttggtgtccaaccggcaagcgaagcgcgcaggccgcaggccggaggcttttccccagagaaaattaaaaaaattgatggggcaaggccgcaggccgcgcagttggagccggtgggtatgtggtcgaaggctgggtagccggtgggcaatccctgtggtcaagctcgtgggcaggcgcagcctgtccatcagcttgtccagcagggttgtccacgggccgagcgaagcgagccagccggtggccgctcgcggccatcgtccacatatccacgggctggcaagggagcgcagcgaccgcgcagggcgaagcccggagagcaagcccgtagggcgccgcagccgccgtaggcggtcacgactttgcgaagcaaagtctagtgagtatactcaagcattgagtggcccgccggaggcaccgccttgcgctgcccccgtcgagccggttggacaccaaaagggaggggcaggcatggcggcatacgcgatcatgcgatgcaagaagctggcgaaaatgggcaacgtggcggccagtctcaagcacgcctaccgcgagcgcgagacgcccaacgctgacgccagcaggacgccagagaacgagcactgggcggccagcagcaccgatgaagcgatgggccgactgcgcgagttgctgccagagaagcggcgcaaggacgctgtgttggcggtcgagtacgtcatgacggccagcccggaatggtggaagtcggccagccaagaacagcaggcggcgttcttcgagaaggcgcacaagtggctggcggacaagtacggggcggatcgcatcgtgacggccagcatccaccgtgacgaaaccagcccgcacatgaccgcgttcgtggtgccgctgacgcaggacggcaggctgtcggccaaggagttcatcggcaacaaagcgcagatgacccgcgaccagaccacgtttgcggccgctgtggccgatctagggctgcaacggggcatcgagggcagcaaggcacgtcacacgcgcattcaggcgttctacgaggccctggagcggccaccagtgggccacgtcaccatcagcccgcaagcggtcgagccacgcgcctatgcaccgcagggattggccgaaaagctgggaatctcaaagcgcgttgagacgccggaagccgtggccgaccggctgacaaaagcggttcggcaggggtatgagcctgccctacaggccgccgcaggagcgcgtgagatgcgcaagaaggccgatcaagcccaagagacggcccgagaccttcgggagcgcctgaagcccgttctggacgccctggggccgttgaatcgggatatgcaggccaaggccgccgcgatcatcaaggccgtgggcgaaaagctgctgacggaacagcgggaagtccagcgccagaaacaggcccagcgccagcaggaacgcgggcgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggaca

SEQ ID NO: 45 - DNA, 7779 bp, Artificial Sequence
  Description of Artificial Sequence; note = synthetic construct
acatggtactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatggcgcaacgcaattaatgtaagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacgagctcggtacccggggatcctctagagtcgacctgcaggcatgcaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc

SEQ ID NO: 46 - DNA, 7780 bp, Artificial Sequence
  Description of Artificial Sequence; note = synthetic construct
acatggtactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatggagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatttgcgcattcacagttctccgcaagaattgattggctccaattcttggagtggtgaatccgttagcgaggtgccgccggcttccatgagctcggtacccggggatcctctagagtcgacctgcaggcatgcaagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgattgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccactgtcgactacgccatcatggcgacagcgcctttcctttgggttctctatatcgggcggatcgtggccggcatcaccggggcgactggggcggtagccggcgcttatattgccgatatcactgatggcgatgagcgcgcgcggcacttcggcttcatgagcgcctgtttcgggttcgggatggtcgcgggacctgtgctcggtgggctgatgggcggtttctccccccacgctccgttcttcgccgcggcagccttgaacggcctcaatttcctgacgggctgtttccttttgccggagtcgcacaaaggcgaacgccggccgttacgccgggaggctctcaacccgctcgcttcgttccggtgggcccggggcatgaccgtcgtcgccgccctgatggcggtcttcttcatcatgcaacttgtcggacaggtgccggccgcgctttgggtcattttcggcgaggatcgctttcactgggacgcgaccacgatcggcatttcgcttgccgcatttggcattctgcattcactcgcccaggcaatgatcaccggccctgtagccgcccggctcggcgaaaggcgggcactcatgctcggaatgattgccgacggcacaggctacatcctgcttgccttcgcgacacggggatggatggcgttcccgatcatggtcctgcttgcttcgggtggcatcggaatgccggcgctgcaagcaatgttgtccaggcaggtggatgaggaacgtcaggggcagctgcaaggctcactggcggcgctcaccagcctgacctcgatcgtcggacccctcctcttcacggcgatctatgcggcttctataacaacgtggaacgggtgggcatggattgcaggcgctgccctctacttgctctgcctgccggcgctgcgtcgcgggctttggagcggcgcagggcaacgagccgatcgctgatcgtggaaacgataggcctatgccatgcgggtcaaggcgacttccggcaagctatacgcgccctagaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtcgaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctc